(19) United States
(12) Patent Application Publication
(10) Pub. No.: US 2025/0025660 A1
(43) Pub. Date: Jan. 23, 2025

(54) SYSTEM AND METHOD FOR ESTIMATING EMOTIONAL VALENCE BASED ON MEASUREMENTS OF RESPIRATION

(71) Applicant: New York University, New York, NY (US)

(72) Inventors: Rose Faghih, New York, NY (US); Revanth Reddy, New York, NY (US); Saman Khazaei, Brooklyn, NY (US)

(21) Appl. No.: 18/778,000

(22) Filed: Jul. 19, 2024

Related U.S. Application Data
(60) Provisional application No. 63/514,825, filed on Jul. 21, 2023.

(51) Int. Cl. A61M 21/00

(52) U.S. Cl. CPC A61M 21/00; A61M 2021/0022; A61M 2021/0072

(57) ABSTRACT

A system for estimating emotional valence continuously based on physiological measurements of respiration activity comprises a respiration sensor, a low-performance computing device configured to acquire the sensor data and estimate an emotional valence state of the wearer an emotional valence estimator, a high-performance computing device configured to provide feedback to the low-performance computing device in order to improve valence estimation and a display to show the estimated valence level. Related methods are also disclosed.

### CROSS-REFERENCE TO RELATED APPLICATIONS

This application claims priority to U.S. provisional application No. 63/514,825 filed on Jul. 21, 2023, incorporated herein by reference in its entirety.

### STATEMENT REGARDING FEDERALLY SPONSORED RESEARCH OR DEVELOPMENT

This invention was made with government support under 2226123 awarded by the National Science Foundation. The government has certain rights in the invention.

### BACKGROUND OF THE INVENTION

Human emotions are complex and difficult to interpret because, unlike physiological signals such as Electrocardiogram (ECG) and Electroencephalogram (EEG), emotions are psychological and immeasurable. In addition, emotion is affected by a number of psychological, physiological, and environmental factors that lead to high subject to subject variability and a lack of an objective measure of emotion (Eaton et al. 2001). Human emotion recognition is a crucial field of study for understanding the mechanisms that drive emotion. It has potential applications in developing actuators to regulate emotions or collecting data in a study where objective measures of emotion are required.

To simplify the diverse spectrum of human emotion, emotions can be modeled on a two-dimensional plane comprising of a measure of valence and arousal for each unique emotion (Russel 1980) (Wickramasuriya et al. 2020). Valence refers to the degree of pleasantness or unpleasantness associated with an emotion, while arousal refers to the intensity of the emotion (Wickramasuriya et al. 2020). In some studies, the emotional valence is the affective quality of interest. Previous studies have found that certain physiological changes occur in response to emotional changes (Sato et al. 2020). These changes can be physiologically measured with common devices such as the EEG, ECG, and respiration belts. Many studies have found success using EEG signals to recognize emotions since an EEG is a direct measure of central nervous system activity that is immediately affected by emotional changes (Schmidt et al. 2001). However, EEGs are impractical for continuous monitoring because of their susceptibility to artifacts and lack of portability (Roy et al. 2021). In comparison, respiration activity is more practical for continuous measurement and can contain information indicative of emotional changes.

Respiration activity is typically measured through a respiration belt around the abdomen. The belt measures changes in the circumference of the abdomen in response to the expansion and contraction of the lungs during a regular breathing cycle. The resulting signal comprises of repeating waves representing the inhalation, exhalation, and breathing cycle of the subject. Respiration differs from other physiological signals since, despite respiration typically occurring involuntarily, it can also be controlled voluntarily. During involuntary respiration, the respiration waveforms are more uniform, where certain qualities of respiration, such as respiration rate and respiration depth, follow a Gaussian distribution around an individual-specific baseline. Voluntary changes in respiration patterns occur in response to an external stimulus. In the absence of confounding factors, an emotional stimulus has been shown to cause changes in respiration patterns (Zhang et al. 2017). For example, deep and fast breathing has been shown to occur in response to excitement, while slow and shallow breathing has been shown to occur in response to negative emotion. These qualities of respiration in addition to the feasibility of data collection make respiration activity an invaluable predictor of emotional state.

Furthermore, emotion is a core aspect of human experience driven by complex neural interactions and environmental stimuli that can be crucial indicators of a person's physical and mental wellbeing (Payne et al. 2003). Understanding emotion has proven to be a difficult task because emotions are ill-defined and highly subjective in nature. The perception of common emotions such as happiness, fear, or excitement are unique to individuals and are incomparable between persons (Dzedzickis et al. 2020). The valence-arousal model is frequently used in research to simplify the spectrum of emotion into defined parameters. Valence describes the positive-negative, or pleasant-unpleasant, aspect of an emotion. Emotions such as joy or excitement are associated with high or positive valence, whereas emotions such as fear and sadness are associated with low or negative valence. Arousal describes the intensity of the emotion. For example, joy and excitement are both associated with positive valence, but vary in intensity and physiological response (Russell 1980). The valence-arousal model allows us to quantify emotion and better relate changes in emotion to physiological processes.

Though a person's current emotion could be influenced by a multiplex of environmental and psychological stimuli, immediate changes in emotion can often be linked to a single stimulus (Eaton et al. 2001). Interactions between the amygdala and hippocampal complex define a specific response to an environmental stimulus. The amygdala receives input from connected sensory processing regions such as the visual cortex to dictate a response to a given stimulus. This response is defined prior to the person's own perception of the emotion but is later modified by their perception (Whalen et al. 1998). The hippocampal complex modulates the behavior of the amygdala depending on memory to define the emotional significance and interpretation of a stimulus. This interaction, however, occurs both ways since memories are associated with previous interpretations of stimuli by the amygdala (Phelps et al. 2015). As a result of these interactions, a physiological response to an emotional stimulus can occur before an individual's perception and modification of the emotional response; thus, making these acute physiological responses important indicators of immediate emotional change.

Additionally, recently wearable device companies have used electrodermal activity and heart rate to predict stress in users, which is related to emotion, but still rely on self-reporting for mood tracking. There are no commercially available solutions that utilize respiration signals for valence estimation. Existing methods have used electroencephalogram signals, but these technologies are limited in their applications because of the impracticality of measuring these signals. In comparison, respiration is practical to continuously observe with advancements in wearable technology and is more ideal for valence estimation tasks.

### SUMMARY OF THE INVENTION

Some embodiments of the invention disclosed herein are set forth below, and any combination of these embodiments (or portions thereof) may be made to define another embodiment.

In one aspect, a system for estimating emotional valence continuously based on physiological measurements of respiration activity comprises a respiration sensor, a low-performance computing device configured to acquire the sensor data and estimate an emotional valence state of the wearer via an emotional valence estimator, a high-performance computing device configured to provide feedback to the low-performance computing device in order to improve valence estimation, and a display to show the estimated valence level.

In one embodiment, at least one of the low-performance computing device and the high-performance computing device comprises a processor and a non-transitory computer-readable medium with instructions stored thereon, which when executed by the processor, perform steps comprising measuring a respiration signal via the respiration sensor, calculating a depth of breath, breathing cycle time, and respiration rate based on the respiration signal, generating a marked point process (MPP) using k-means grouping of the calculated depth of breath, breathing cycle time, and respiration rate, estimating a valence level based on the MPP, and displaying the estimated valence level.

In one embodiment, the respiration sensor comprises a respiration belt, a camera, an electroencephalogram (EEG), or an electrocardiogram (ECG).

In one embodiment, the system further includes a stimulation device configured to perform an intervention.

In one embodiment, the intervention comprises a vibration.

In one embodiment, the intervention comprises an electrical stimulation.

In one embodiment, the display is configured to display an intervention suggestion.

In one embodiment, the intervention suggestion comprises instructions to perform a breathing exercise, playing music, or instructions to administer medication.

In another aspect, a method for estimating emotional valence continuously based on physiological measurements of respiration activity comprises measuring a respiration signal via a respiration sensor, calculating a depth of breath, breathing cycle time, and respiration rate based on the respiration signal, generating a marked point process (MPP) using a k-means grouping algorithm on the calculated depth of breath, breathing cycle time, and respiration rate, estimating a valence level based on the MPP, and displaying the estimated valence level.

In one embodiment, the method further includes improving the valence estimation via feedback.

In one embodiment, the step of generating the MPP comprises identifying high and low valence events in the respiration signal.

In one embodiment, the high and low valence events are identified by comparing features extracted from each breath to their expected behavior during no emotional response.

In one embodiment, the respiration signal comprises a waveform including inhalation amplitude, exhalation amplitude, inhalation time, and exhalation time.

In one embodiment, the depth of breath is the difference between the amplitude of respiration measured at the end of inhalation and the amplitude measured at the start of inhalation, and the rate of inhalation comprises breath amplitude divided by the time of inhalation.

In one embodiment, the algorithm comprises an unsupervised algorithm.

In one embodiment, the unsupervised algorithm comprises k-means clustering.

In one embodiment, the method further includes administering an intervention when a negative valence is estimated.

In one embodiment, the intervention comprises instructions to perform a breathing exercise, music, or instructions to administer medication.

In one embodiment, the intervention is automated.

In one embodiment, the intervention comprises a vibration or an electrostimulation.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A shows a sample valence estimation using respiration measured with a respiration belt. A sample respiration amplitude recording for a single breath is shown. Red circles indicate the local minima and maximum detected for feature extraction corresponding to the start of inhalation, the end of inhalation, and the end of exhalation. The depth of breath is defined as the difference between the amplitude of respiration measured at the end of inhalation and the amplitude measured at the start of inhalation. Rate of inhalation is defined as the breath amplitude divided by the time of inhalation. These features are then used to generate a marked point process (MPP) based on k-means grouping of breaths. This MPP can then be used to predict a valence level in near-real time based on a previous history of breaths.

FIG. 1B shows an overview of the system.

FIG. 1C shows a closed loop control of valence estimation including the system.

FIG. 2 shows a valence estimation pipeline.

FIG. 3 shows a valence estimation method.

FIG. 4 shows a systems representation of valence estimation.

FIG. 5 shows an overview of the hardware configuration.

FIG. 6 shows an example of detected locations in a sample respiration signal. Red points represent the detected respiration peaks at the end of inhalation. Green points represent the start of inhalation. Blue points represent the end of exhalation.

FIG. 7 shows a sample binary process generated from a respiration signal. Respiration amplitude, respiration derivative, and cycle time are shown. The green markers indicate the presence of a high valence indicative event. The red lines indicate the thresholds for the features to be considered a high valence indicative event. The last plot depicts the generated binary point process based on the events of the individual features. A point is generated if an event is detected in both the amplitude and derivative features or the cycle time feature.

FIG. 8 shows valence state estimation performed on subject 1. The subplots, in order, represent the pre-processed respiration signal, the binary point process, the estimated valence state, the predicted valence index, and the normalized and interpolated self-reported valence rating. The red intervals indicate the periods in which video clips are shown to subjects. High valence periods are shown in green for both valence index and self-reported rating. Low valence periods are indicated in red.

FIG. 9A is a table of high and low valence estimation accuracy for all subjects. Participant number corresponds to the numbering of subjects in the MAHNOB-HCI database.

FIG. 9B is a table of results from the Wilcoxon Rank Sum Test Participant Number corresponds to the numbering of subjects in the MAHNOB-HCI database. The p-value corresponds to a right-tailed statistical test, testing whether the median difference between the high and low valence estimates are larger than zero.

FIG. 10A shows a sample respiration wave measured with a respiration belt corresponding to a single breath. Red circles indicate the local minima and maximum detected for feature extraction corresponding to the start of inhalation, the end of inhalation, and the end of exhalation. The depth of breath is calculated as the difference between the amplitude of respiration measured at the end of inhalation and the amplitude measured at the start of inhalation. Rate of inhalation is calculated by dividing the depth of breath by the total inhalation time.

FIG. 10B shows K-means grouping of individual breaths along two features: breath amplitude and inhalation rate red points represent breaths likely to correspond to low valence events, whereas green points correspond to high valence events. The high density of breaths containing normalized breath amplitude values close to 0 are also highlighted and removed before labeling as they likely correspond to neutral valence.

FIGS. 11A-11D show marked point process generation and state estimation for high and low valence for Participants 1 and 3. The plots represent the preprocessed respiration signal, the generated marked point process, the high or low valence state, the high or low valence index, and the normalized self-reported valence ratings in that order. Confidence intervals are shown in the valence state plot. The extremes of the high and low valence indices are shown in green and red indicating locations of very high (green) or very low (red) valence.

FIGS. 12A-12B show marked point process generation and combined valence index estimation for two participants. The preprocessed respiration signal, high (green) and low (red) valence events, high and low valence indices, combined valence index, and interpolated, normalized self-reported ratings are shown. Low valence events are visualized as negative pulses to better observe the negative correlation between high and low valence events. Intervals in which video clips are shown to participants are shown in blue.

FIGS. 13A-13B show high and low valence period estimation in identifying high and low valence periods throughout the signal recording. High valence periods are indicated in green and low valence periods are indicated in red. These periods are only identified during video clip viewing as indicated by the pink intervals.

FIG. 14 is a table of accuracy for predicting high and low valence events for all participants. Participant number corresponds to the participant number in the MAHNOB-HCI database.

FIGS. 15A-15AH show marked point process generation and state estimation for high and low valence for all participants. The plots represent the preprocessed respiration signal, the generated marked point process, the high or low valence state, the high or low valence index, and the normalized self-reported valence ratings in that order. Confidence intervals are shown in the valence state plot. The extremes of the high and low valence indices are shown in green and red indicating locations of very high (green) or very low (red) valence.

FIGS. 16A-16W show valence estimation results for all participants. The preprocessed respiration signal, high valence MPP (green), low valence MPP (red), high valence index (green), low valence index (red), combined valence index, and normalized self-reported ratings are shown. The high and low valence MPPs are represented as positive and negative pulses on the same axis for visualization but are both positive values from feature extraction. The combined valence index is calculated from the high and low valence indices and plotted alongside the self-reported ratings for comparison. Periods in which video clips are shown corresponding to the trials are shown in blue. Each participant is shown a set of 20 video clips in a randomized order. Two participants (7 and 11) are missing portions of the recording and video clips.

FIG. 17A-17Q show high and low valence period estimation for all participants for identifying high and low valence periods throughout the signal recording. High valence periods are indicated in green and low valence periods are indicated in red. These periods are only identified during video clip viewing as indicated by the pink intervals.

FIGS. 18A-18B show box plots of valence event parameters during high and low periods for all participants. Mean amplitude and number of high and low valence events are measured in either period, and the results are aggregated over 23 participants. High and low valence periods are defined based on a cutoff of 0.5 in participants' normalized and rescaled self-reported valence ratings. ** and * denotes statistical significance with p-value less than 0.05 and 0.15, respectively. Outliers beyond 1.5 times the interquartile range from the median value are not shown but included for analysis. The results indicate that both high and low events are more likely to occur and have larger amplitudes in their respective periods, though they differ in which feature offers more information.

FIG. 19 shows box plots of valence index during high and low periods for four participants. For both types of valence periods, the valence index is measured at each time bin to compare the effectiveness of the algorithm in discerning high and low valence periods. High and low valence periods are determined based on a threshold of 0.5 for the normalized self-reported ratings.

FIGS. 20A-20H show box plots of valence index during high and low periods for all participants. For both types of valence periods, the valence index is measured at each time bin to compare the effectiveness of the algorithm in discerning high and low valence periods. High and low valence periods are determined based a threshold of 0.5 for the normalized self-reported ratings. Outliers are indicated if they are beyond 1.5 times the interquartile range from either quartile. For the participants whose high and low valence events appeared to be interchanged, they were reassigned before plotting.

FIG. 21 depicts an exemplary computing environment in which aspects of the invention may be practiced.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods for estimating emotional valence based on measurements of respiration. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, and +0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are systems and methods for estimating emotional valence based on measurements of respiration.

Currently, there is no objective measure of emotional valence. For tasks such as multimedia recommendation, companies rely on self-reporting an emotional response (like, dislike, etc.). These self-reported ratings are subject to heavy bias and are generally unreliable predictors of an emotional response. Using a method for continuous valence tracking would allow one to know exactly at which times someone felt a positive or negative emotion provided the person's respiration signal is measured. Other methods for valence estimation classify whole signals to high and low valence instead of a continuous estimate, or they categorize the prediction into emotions (happy, sad, excited etc.) rather than a numerical value. This makes the method more useful for applications in continuous monitoring of patient's moods where applicable. For example, it could be valuable information to know if someone suffering from specific mental disorders (such as PTSD, Schizophrenia, major depressive disorder, or bipolar disorder) is experiencing an extended period of low valence. In addition, this technology can have applications in improving performance and productivity. It can be used in a professional setting to alert people who are experiencing extended periods of negative valence which could impact their performance and quality of work. In addition, since respiration can be manually controlled, the device can alert users to modulate their breathing and control their own valence levels during a low valence event.

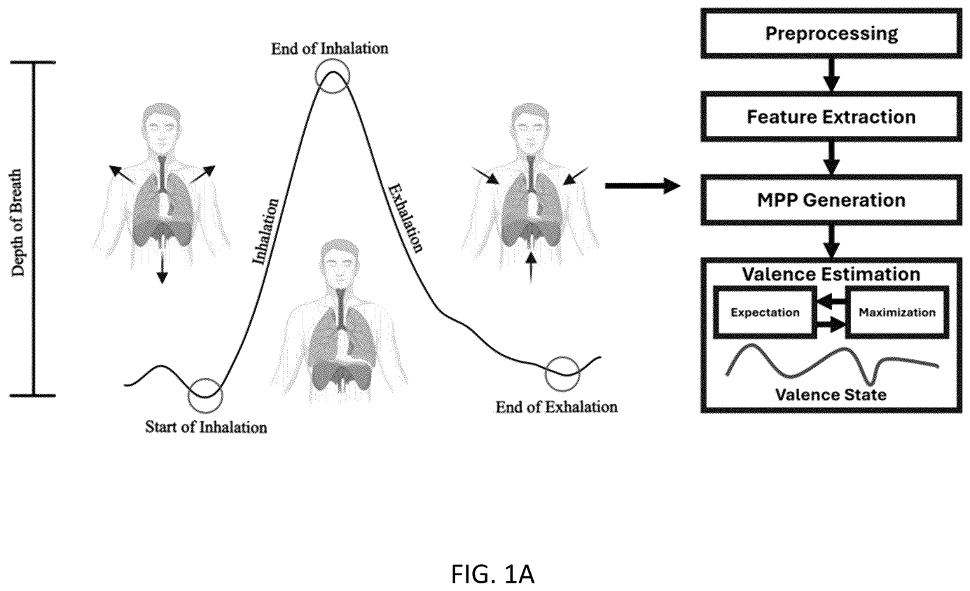

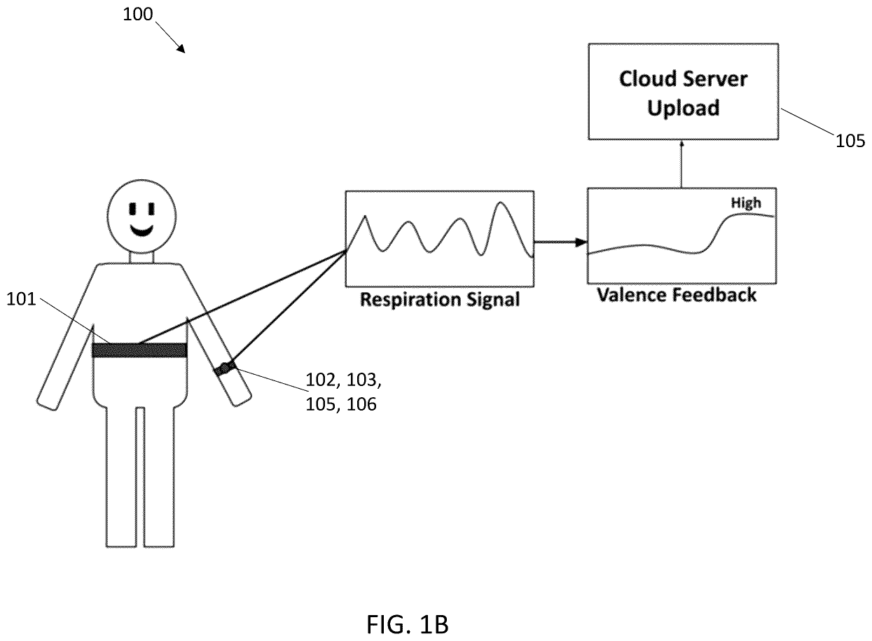

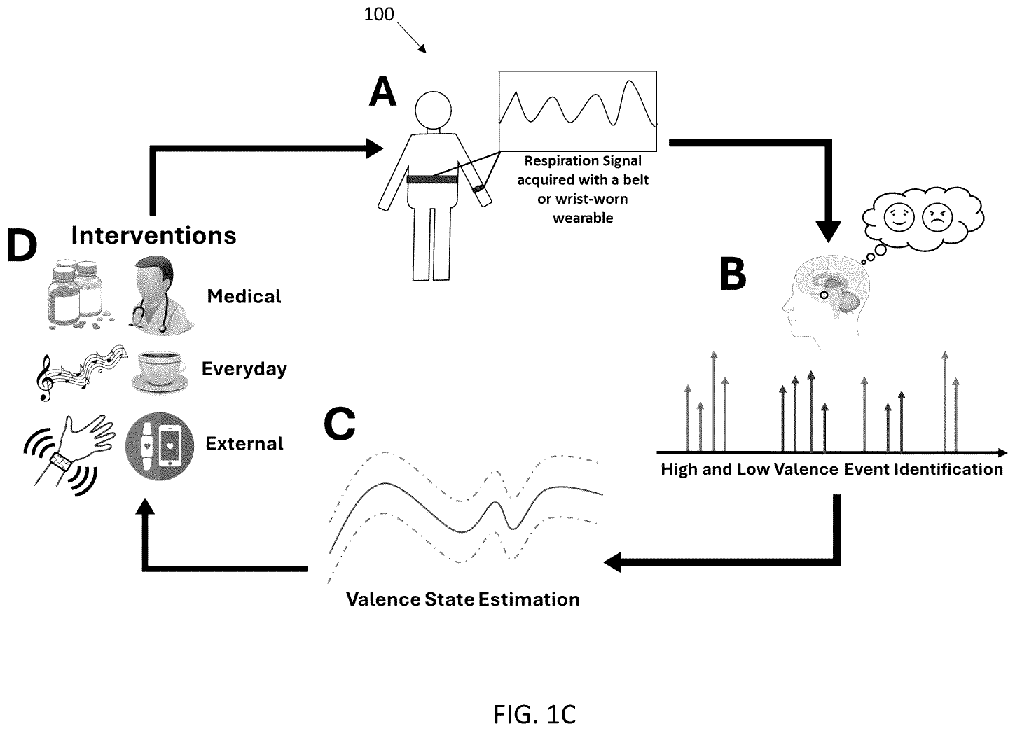

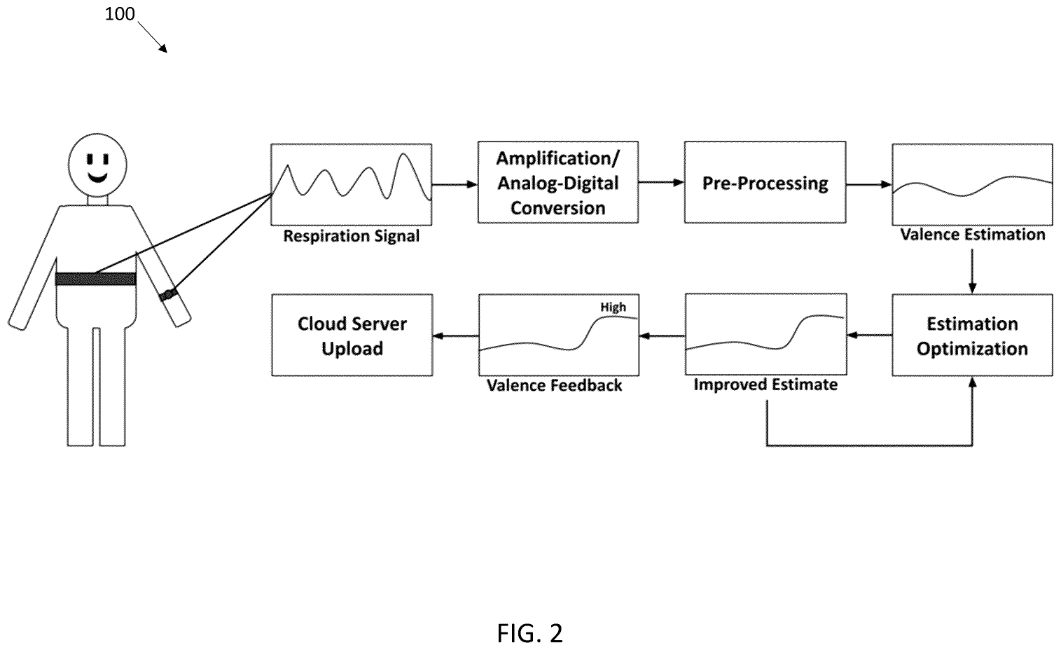

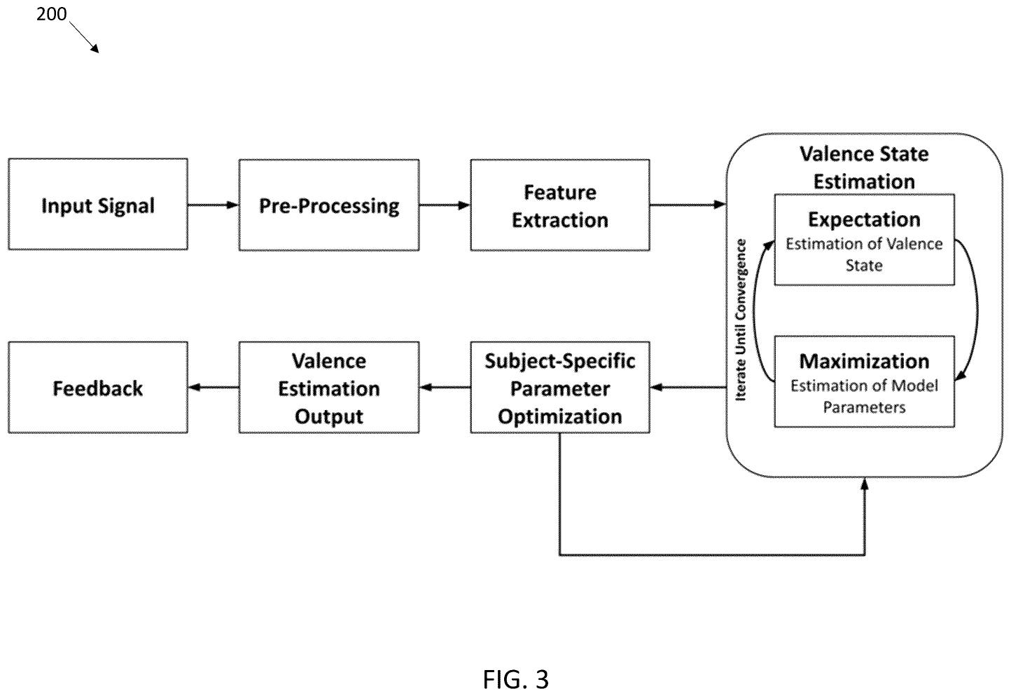

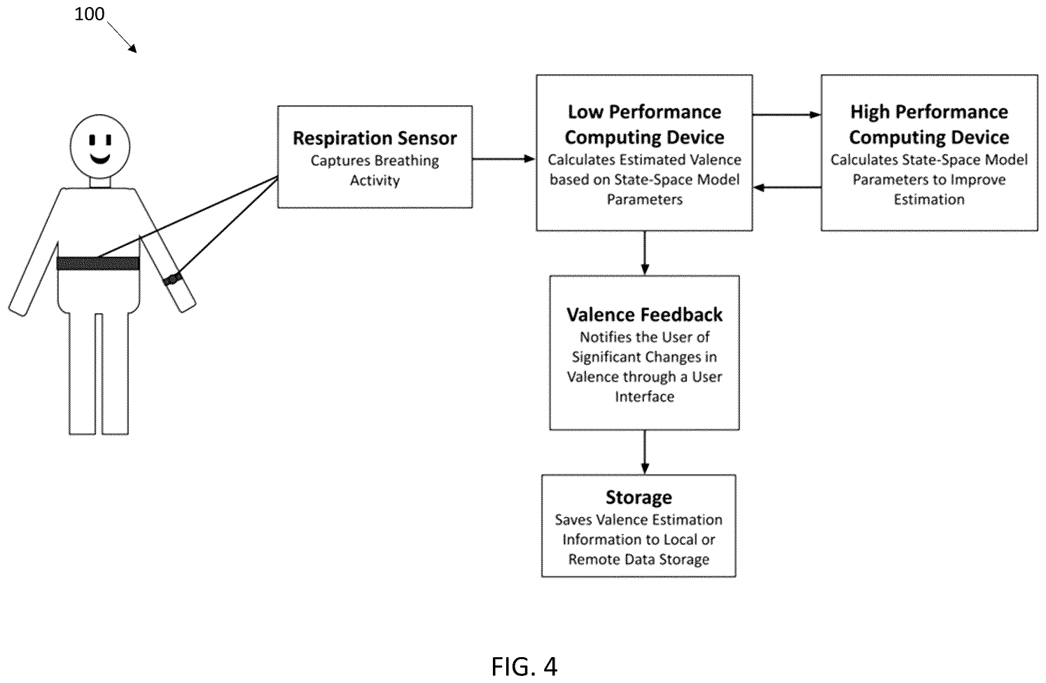

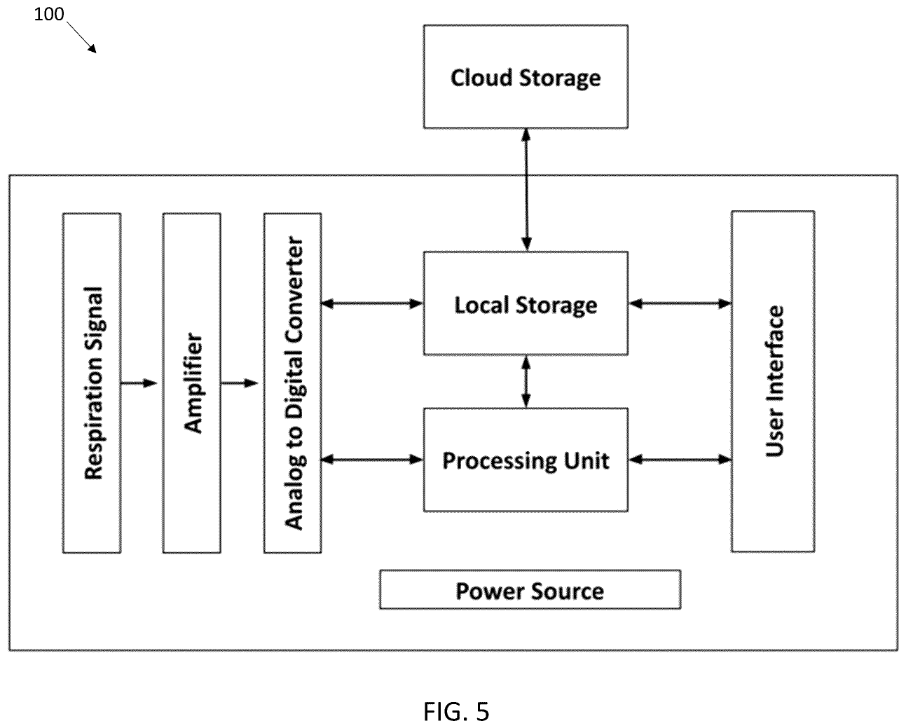

Referring now to FIGS. 1-5, an exemplary system 100 and method 200 for estimating emotional valence continuously based on physiological measurements of respiration activity is shown. Valence refers to the degree of pleasantness or unpleasantness associated with emotion.

In some embodiments, a system 100 for estimating emotional valence continuously based on physiological measurements of respiration activity comprises a respiration sensor 101, a low-performance computing device 102 communicatively connected to the respiration sensor 101 configured to acquire the sensor data and estimate an emotional valence state of the wearer an emotional valence estimator 103, a high-performance computing device 104 communicatively connected to the low-performance computing device 103 configured to provide feedback to the low-performance computing device 103 in order to improve valence estimation, and a display 105 to show the estimated valence level.

In some embodiments, the system comprises five main components: a respiration sensor 101, a low-performance computing device 102 to acquire the sensor data and estimate an emotional valence state of the wearer, an emotional valence estimator 103, a high-performance computing device 104 to provide feedback to the low-performance device in order to improve estimation, and a display 105 (such as on the wearable or a smart-phone) to show the estimated valence level.

In some embodiments, the respiration sensor 101 measures physiological information regarding the person's breathing activity. Any suitable respiration sensor can be employed, including a respiration belt (by either measuring strain, impedance or movement during breathing), air flow sensors, an electrocardiogram (ECG), a photoplethysmogram (PPG), a microphone, or a camera.

In some embodiments, the low-performance computing device 102 reads this information regarding respiration activity. In some embodiments, the low-performance computing device 102 comprises a processor and a non-transitory computer-readable medium with instructions stored thereon, which when executed by the processor, may perform steps comprising measuring a respiration signal via the respiration sensor 101, calculating a depth of breath, breathing cycle time, and respiration rate based on the respiration signal, generating a marked point process (MPP) using k-means grouping of the calculated depth of breath, breathing cycle time, and respiration rate, estimating a valence level based on the MPP, and/or displaying the estimated valence level. In some embodiments, the emotional valence estimator 103 outputs an estimate of the emotional valence based on the respiration signal. In some embodiments, the low-performance computing device 102 comprises a microcontroller, such as an Arduino, which then outputs information to the high-performance computing device. Alternatively, the role of the low-performance device can also be performed by a high-performance computing device such as a smart watch, smart phone, or a computer.

In some embodiments, the high-performance computing device 104 analyzes the respiration activity information to improve the quality of the valence estimation. In some embodiments, the high-performance computing device 104 comprises a processor and a non-transitory computer-readable medium with instructions stored thereon, which when executed by the processor, may perform steps comprising measuring a respiration signal via the respiration sensor 101, calculating a depth of breath, breathing cycle time, and respiration rate based on the respiration signal, generating a marked point process (MPP) using k-means grouping of the calculated depth of breath, breathing cycle time, and respiration rate, estimating a valence level based on the MPP, and/or displaying the estimated valence level. In some embodiments, the high-performance computing device 104 comprises a smart watch, smart phone, or computer capable of high-speed processing. The respiration information can also be processed using remote computing devices such as a server or cloud-based processing unit, which can directly receive the respiratory signal from the low-performance computing device embedded into the sensor.

In some embodiments, the system 100 further includes a stimulation device 106 configured to perform an intervention. In some embodiments, the intervention comprises a vibration and/or electrical stimulation.

In some embodiments, the display 105 visualizes the valence estimation results to the user. In some embodiments, the display 105 is configured to display an intervention suggestion such as instructions to perform a breathing exercise, playing music for the user, instructions to administer medication, and/or any other suitable interventions.

In some embodiments, a method 200 for estimating emotional valence continuously based on physiological measurements of respiration activity comprises providing the system 100, measuring a respiration signal via the respiration sensor 101, calculating a depth of breath, breathing cycle time, and respiration rate based on the respiration signal, generating a marked point process (MPP) using a k-means grouping algorithm on the calculated depth of breath, breathing cycle time, and respiration rate, estimating a valence level based on the MPP, and displaying the estimated valence level. In some embodiments, the algorithm comprises an unsupervised algorithm. In some embodiments, the method 200 further includes improving the valence estimation via feedback.

In some embodiments, the step of generating the MPP comprises identifying high and low valence events in the respiration signal. In some embodiments, the high and low valence events are identified by comparing features extracted from each breath their expected behavior during no emotional response.

In some embodiments, the respiration signal comprises a waveform including inhalation amplitude, exhalation amplitude, inhalation time, and exhalation time. In some embodiments, the depth of breath is the difference between the amplitude of respiration measured at the end of inhalation and the amplitude measured at the start of inhalation, and the rate of inhalation comprises breath amplitude divided by the time of inhalation.

In some embodiments, the method 200 further includes administering an intervention when a negative valence is estimated. In some embodiments, the intervention comprises instructions to perform a breathing exercise, music, or instructions to administer medication. In some embodiments, the intervention is automated. In some embodiments, the intervention comprises a vibration and/or an electrostimulation.

In some embodiments, the method 200 for estimating emotional valence using respiration activity involves measuring respiration activity using a respiration sensor 101, estimating the emotional valence state based on the respiration information via the valence estimator 103, receiving feedback to improve estimation, outputting the estimated valence state to the user via the display 105, and providing feedback based on the results of estimation.

In some embodiments, the method 200 for estimating the emotional valence based on respiration information is as follows. First, the location of events that indicate a high valence emotional response in the respiration signal are identified. This is done by comparing features extracted from each breath (depth of breath, respiration rate, and breathing cycle time) with their expected behavior during no emotional response. The expected behavior of these features is measured assuming that the respiration features follow a normal distribution in the absence of an emotional stimulus. Each breath is marked as indicative of high valence if the associated features are significantly deviating from the normal distribution. The probability of these high valence events occurring is related to a continuous estimate of valence using a state-space model. The state-space model predicts an unknown state given an input signal and parameters describing the relation between the input and unknown valence state. The result is a continuous numerical value indicating the probability that the subject is experiencing high valence. This method 200 is then repeated for low valence events by marking events corresponding to decreases in depth of breath that would be indicative of low valence. A combined valence estimate is then generated from the high and low valence levels. To test the accuracy of valence estimation, the estimates were compared with the self-reported valence levels of the subjects as detailed below.

In some embodiments, continuous estimate of valence provides a user with information regarding a person's emotions over time. Depending on the application, it could be important to detect high or low valence periods throughout a person's day. For example, in terms of multimedia recommendation, it could be important to understand at which points in the media the person felt a very pleasant emotion. This eliminates the need for self-reporting emotions, which can be prone to bias. On the other hand, identifying low valence periods can be important in applications related to mental health and wellbeing. There is great value in understanding the negative emotions felt by someone suffering from certain mental illnesses, such as major depressive disorder or manic depression. In these cases, it could be possible to introduce feedback to alleviate the negative emotions and their effects on the wellbeing of the individual. Several interventions are discussed below that can be applied based on the negative emotional valence estimates including medical interventions, everyday interventions, and external stimulation.

### Medical Intervention

In certain cases, such as major depressive disorder, understanding the incidence of negative emotions over time can be of great value for medical professionals in designing a proper treatment for individuals. Treatment options can include psychotherapy and pharmacotherapy, both of which can benefit from a more objective measure of the person's emotional behavior (see Kupfer et al. 2012). Pharmacological interventions can involve both short-term and long-term (maintenance) treatments for those suffering from depressive disorders. Certain medications, such as fluoxetine, a selective serotonin reuptake inhibitor (SSRI), have relatively short half-lives ranging from 1-4 days (see Hiemke et al. 2000), which may be useful in preventing remission into clinical depression. For example, medical professionals can monitor a person's emotional wellbeing throughout the day and administer fast-acting antidepressants to alleviate symptoms of the patient. Understanding the daily patterns of pleasant and unpleasant emotion can also be useful in the long-term treatment, or maintenance, of those suffering from certain mental illnesses. Long-term treatment, either in the form of psychological or pharmacological therapy, have been shown to have high rates of recurrence (see Keller 1999). A continuous valence estimator can detect recurrence of depression, or other mental illnesses associated with negative emotions, and alert the respective professionals when medical intervention is necessary.

### Everyday Interventions

Though medical intervention may be necessary in certain cases, everyday interventions have proven to be effective in alleviating negative emotions. Safe everyday interventions can include simple tasks that pose no risk and can be completed by the person themselves. This can include activities such as breathing exercises, playing music, or even drinking a cup of coffee. Unlike medical interventions, these can be performed by healthy individuals experiencing short-term negative emotions, such as anxiety or panic. Self-regulation of breathing has been shown to be a reliable treatment of anxiety, panic, and negative emotions (see Jerath et al. 2015) and can be administered by the person themselves. A valence estimator can alert users when they are experiencing negative emotions through alterations in their respiratory patterns and prompt them to perform breathing exercises to alleviate their symptoms. These breathing exercises can include deep breathing, which has been shown to reduce perceived stress and anxiety (see Perciavalle et al. 2017), as well as breathing at a consistent pace (6 breaths per minute), which has been shown to improve both heart rate variability and mood (see Steffen et al. 2017). Breathing exercises are particularly effective since they can be done in a natural setting, such as sitting at a workstation or during a meeting, which do not require the user to change their environment. Similarly, music therapy has been shown to be an effective treatment for depression and other affective disorders (see Aalbers et al. 2017) (see Koelsch et al. 2010). Safe interventions are particularly useful since they can be performed anywhere and offer very minimal risk to the person performing them. In addition, in previous studies performed (see Khazaei et al. 2024) (See Fekri Azgomi et al. 2023), these safe interventions were found to have an effect on the performance and arousal of individuals performing memory tasks, suggesting potential application in workplace and educational optimization. These interventions may similarly affect the emotional valence of the individuals and could improve mood and, thus, productivity.

### External Stimulation

Mental health disorders such as depression and anxiety can be addressed by altering emotional status via various forms of external stimulation. These stimulations include but are not limited to tactile and electrical feedback, which can be applied within everyday life settings. Tactile wearables can generate a paired vibration pattern within the body and may regulate the emotional status (see Hiraba et al. 2014). Electrical stimulation is another form of external feedback that can shift brain function via electricity and impact the underlying emotion. Particularly, previous studies have noted this form of stimulation as a safe approach that may positively impact emotion (see Tyler et al. 2015).

FIG. 1C describes a closed-loop control of the valence estimation system. (A) describes the measurement of respiration taken from individuals. These measurements can be taken using a respiration belt, which directly measures the expansion and contraction of the abdomen, or other sensors from which respiration can be derived from, including PPG signals (see Nilsson 2013) or even cameras (see Rehouma et al. 2020). (B) describes the process of high and low valence event identification in the signal. The amygdala defines a positive or negative valence response to a given stimulus which then affects the respiratory pattern of the individual. Each breath is labeled as high, low, or neutral valence indicative. In (C), the process of valence estimation given the occurrence of high and low (positive and negative) events is shown. The valence estimation provides a continuous numerical value for valence which can be used to identify high and low periods of valence. (D) depicts several methods of intervention to alleviate the symptoms associated with low valence. This can include medical intervention in the case of mental illness but can also include everyday interventions, such as listening to music or drinking coffee, which can improve negative emotions. Also shown is external stimulation such as tactile or electrical stimulation that may improve negative valence. In addition, warnings of low valence can be communicated to the user and/or medical professionals via smartphone to administer any effective intervention strategies.

To summarize, the illustrated forms of external feedback can be implemented within the closed-loop architecture to regulate emotional valence and set that to the range of interest. As an example of such implementations, one can refer to the regulated stress response via transdermal neuromodulation (see Tyler et al. 2015) and reduced stress-related anxiety resulting from bilateral alternating somatosensory stimulation (a form of tactile stimulation) (see Cesar Pinto Leal-Junior et al. 2019). The valence estimator system 100 can play a crucial role in determining the timing and intensity of such external stimulations. Thus, in one embodiment, the system 100 can be used to estimate a valence state and suggest and/or apply an intervention either automatically and/or manually in response to the estimated valence state. Further, the result of the intervention can be monitored by the system 100 to determine if additional and/or different interventions should be suggested and/or applied. This loop can be performed continuously if necessary.

In a further embodiment, valence estimation can be utilized to provide user recommendations for media consumption, meal suggestions, or similar based on determined valence states.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore specifically point out exemplary embodiments of the present invention and are not to be construed as limiting in any way the remainder of the disclosure.

In the emotion recognition paradigm, clinical studies and available datasets collect physiological signals from subjects while emotions are elicited using visual and auditory stimuli. Multiple experiments were performed to test the above-described systems and methods. In one of the studies, the Multimodal Database was used for Affect Recognition and Implicit Tagging (MAHNOB-HCI) database (Soleymani et al. 2012). The dataset comprises of physiological measurements taken from subjects in response to 20 movie and video clips meant to elicit specific emotions. The valence and arousal levels of subjects were self-reported after each clip is shown. The authors of the dataset were able to achieve a valence and arousal classification accuracy of 45.5% and 46.2%, respectively, using a support vector machine classifier with features derived from physiological signals including galvanic skin response, ECG, respiration, and skin temperature. Similarly, other studies have achieved high accuracy in emotion recognition using machine learning and deep learning methods. However, these studies do not provide a continuous measure of valence or consider underlying neural stimuli (Sato et al. 2020) (Zhang et al. 2017). In this study, a state-space model similar to prior models was developed to relate emotional valence to deviations from involuntary respiration patterns.

For each respiration cycle, the depth of breath, derivative of respiration amplitude (i.e. rate of respiration), and total respiration cycle time were measured. The deviations from a Gaussian distribution in these features are represented as binary point processes for each feature, in which impulses represent events indicative of high valence over time. The point process approach is a common method to model neural spiking activity and represents the underlying neural stimuli driving the involuntary changes in respiration immediately after an emotional stimulus. Several studies have used a similar approach to relate underlying neural stimuli to an unobserved process in the brain (Wickramasuriya et al. 2020) (Shanechi et al. 2012) (Shanechi et al. 2014). A point process approach is ideal for respiration signals since features are measured at discrete times corresponding to the temporal location of each breath. In particular for each breath, a point is made to process by assigning a value of one if both the respiration amplitude and respiration rate probabilities or the respiration cycle time probabilities are below a specified threshold probability value, or zero otherwise. An Expectation-Maximization (EM) algorithm is used to decode the valence state in relation to the high valence indicative events. Eventually, the decoded valence state is evaluated given the self-reported valence ratings from subjects.

### Dataset

The MAHNOB-HCI dataset comprises of an emotion recognition experiment and an implicit tagging experiment. The experiment involved data collection from 27 subjects. Subjects were shown 20 video clips meant to elicit different emotional responses. During each trial period where a video clip is shown, subjects are shown a neutral clip meant to induce no emotion, the emotional clip, and then self-assess their emotions. During the experiment, several physiological signals are measured such as ECG, EEG, galvanic skin response, eye gaze, respiration, and skin temperature. For each subject, the physiological measurements are recorded over an approximately 50-minute period in which the video clips are shown in a randomized order. From the 27 subjects, subjects with incomplete recordings from technical difficulties, significant measurement noise, and those who did not consent to publishing were excluded. Analysis on the 14 remaining subjects was performed.

### Pre-Processing

The respiration signal is susceptible to noise, motion artifacts, and wandering baselines that can impact analysis. Similar to previous methods, noise and wandering baselines are corrected using a Butterworth bandpass filter with cutoff frequencies of 0.2 and 2 Hz (Castegnetti et al. 2017). This filter corrects the baseline of the signal to zero and removes polynomial trends from the respiration signal. The cutoff frequencies are selected to remove high frequency sensor noise as well as potential low frequency artifacts. Motion artifacts are present in the respiration signals as irregular increases in respiration amplitude in the order of 5-10 times the magnitude of a typical breath in the same subject. Since the amplitude of the normal signal differs greatly from the signal impacted by motion artifacts, a moving average filter is implemented on the signal to determine the locations at which a motion artifact is likely occurring based on the magnitude of the signal exceeding the feasible range of amplitudes. Once these locations are detected, they are removed from the original signal to limit the impact of motion artifacts on the analysis.

### Feature Extraction and Point Process Generation

Features from the respiration signal should be extracted based on the underlying physiology such that they can be used to infer the valence level. Based on empirical evidence and prior work (Sato et al. 2020) (Zhang et al. 2017), it was decided to monitor the behavior of respiration amplitude, rate of respiration, and total cycle time of respiration. The MATLAB findpeaks function was used to detect the local maxima throughout the respiration signal. The local minima found before and after each peak correspond to the beginning of inhalation and ending of exhalation, respectively. Respiration amplitude is measured as the difference between the amplitude of the peak and the amplitude of respiration when inhalation begins. The total cycle time of respiration is defined as the length of time between the beginning of inhalation and the end of exhalation for a single breath. Here, the rate of respiration refers to the derivative of the respiration amplitude signal. The derivative of the signal represents the rate at which inhalation and exhalation is occurring. For each respiration wave, the respiration amplitude, cycle time, and number of times the derivative of respiration exceeds a threshold within a 2.5 second window before the wave are measured. The number of times the derivative crossed the threshold was also measured to remove extraneous derivative peaks from noise. A 2.5 second interval was selected to allow us to consider the rate of exhalation for the previous breath and the rate of inhalation for the current breath while ignoring the rate of inhalation of the previous breath, which has already been considered. In other words, a 2.5 second interval is the largest time interval in which no more than one pulse can occur.

A binary point process is created based on the features measured at each respiration wave. Assuming that respiration amplitude, rate of respiration, and cycle time are all Gaussian distributed in the absence of an emotional stimulus, significant deviations from the Gaussian distribution can be seen as a response to a stimulus. As a result, for each respiration wave, the probability of a measured value occurring for each feature is calculated using the Gaussian cumulative distribution function. For each respiration wave, a binary impulse is generated if both the respiration amplitude and derivative probabilities or the cycle time probabilities are below a specified threshold probability value. The amplitude and derivative features are considered together since they contain overlapping information. The derivative feature consideration removes peaks that are impacted by artifacts or noise, which would not contain the rise in amplitude associated with inhalation before. The cycle time is considered separately from these features because it contains temporal information measured from the raw respiration signal. The thresholds are unique for each feature, are subject-specific, and have been determined empirically. Specific thresholds are required to consider the highly subjective emotional responses of each individual. For example, a subject who is more susceptible to physiological changes to emotion may show a 50% increase in respiration amplitude, whereas a less susceptible subject may only show a 30% increase. In other words, the skewness from the Gaussian distribution in the presence of an emotional stimulus likely depends on the intensity of the subject's specific physiological response.

### State-Space Model

The state-space model relates the binary point process of high valence indicative impulses to a predicted valence state of the subject (Wickramasuriya et al. 2018). First the time axis of the respiration signal is binned into 2.5 second duration intervals. Using the point process generated based on the extracted features, each bin is assigned a value of 1 or 0 based on the presence of a peak. Using the state-space model for point-processes (Smith et al. 2005), a continuous valence level is estimated throughout the duration of the signal. The state-space model comprises of two equations, a state equation describing the latent valence state and an observation equation relating the observed binary pulses to the state equation. It was assumed that a Gaussian state equation best describes the latent state, and a Bernoulli probability model describes the presence of peaks in individual bins as shown in (Wickramasuriya et al. 2020) (Wickramasuriya et al. 2019) (Wickramasuriya et al. 2018). The data was analyzed from the perspective of an ideal observer, having knowledge of the entire signal, to estimate the valence state at each bin. The valence state equation is described as a random walk-through time as follows:

$$\\ \begin{matrix}{z}_{k} = {z}_{k - 1} + {∈}_{k} & EQ . ( 1 )\end{matrix}$$

where ϵₖ˜N (0,σ_{ϵ}²) is the process noise.

Define the total number of bins as K and index each bin as k=1, 2, 3 . . . . K, where K is the duration of the entire respiration signal divided by the bin size. For the observation equation, sₖ is used to represent the presence of a peak in each bin. sₖ follows a Bernoulli distribution where sₖ=1 if bin k contains a peak and sₖ=0 in the absence of a peak. Define q as the probability of a peak occurring in bin k. For the state equation, define zₖ as the latent valence state at bin k. Since the changes in respiration pattern are indicative of changes in valence state, the probability qₖ depends on the state zₖ. Given an arbitrary value of valence state zₖ, the observation equation describes the probability of observing sₖ using a Bernoulli distribution as such P(sₖ|qₖ)=qₖ^{s}^{ₖ}(1−qₖ)^{1-s}^{ₖ}, where qₖ is defined by the logistic equation (Wickramasuriya et al. 2020) (Wickramasuriya et al. 2019) (Wickramasuriya et al. 2018):

$$\\ \begin{matrix}{q}_{k} = \frac{1}{1 + {e}^{{}_{} - ( α + {z}_{k} )}} & EQ . ( 2 )\end{matrix}$$

The value of α depends on the random probability that a peak occurs in a bin at the start of the experiment. It can be estimated using (2) by assuming z₀=0 and finding q₀ empirically by calculating the proportion of high valence events to all breaths.

Based on the binned point process, S_{1:K}={s₁, s₂, . . . , s_{K}}, was observed which indicates the presence of a peak at each bin. The goal is to estimate the valence state Z={z₁, z₂, . . . , z_{K}} and σ_{ϵ}² in order to estimate qₖ for each bin. Since z is unobserved and σ_{ϵ}² is a parameter, one can use the expectation maximization algorithm to estimate both z and σ_{ϵ}².

### Valence State Estimation

The estimation step contains a filter algorithm that calculates the valence state estimate of the subject and a fixed interval smoothing algorithm to refine the estimate based on the ideal observer's perspective. The filter algorithm estimates the valence state z_{k|k} given S_{1:k}, with σ_{ϵ}² being replaced by its maximum likelihood estimate. The fixed interval smoothing estimates z_{k|K} given S_{1:K}, again with the maximum likelihood estimate of σ_{ϵ}². A Gaussian approximation was used for the point process observations to design the filter (Smith et al. 2005) (Smith et al. 2004). Define q_{k|k} as the probability of a peak occurring given observations until bin k and q_{k|k} as the probability of a peak occurring given all observations throughout the duration of the signal. The expectation maximization algorithm is further detailed in the following sections.

### Expectation Step.

Given S_{1:K}, zₖ, σ_{ϵ}^{2(l)}, and z₀^{l}, the expectation was computed of all data log likelihood at iteration/+1, where/is the iteration number of the algorithm.

Forward Filter: The state z_{k|k} and the variance σₖ²_{|k}, given σ_{ϵ}^{2(l)}, and z₀^{l} were estimated using a recursive filter. This algorithm is derived from the maximizing posterior probability of the Kalman filtering algorithm as described in (Mendel 1995) (Brown et al. 1998). The algorithm is described as:

$${z}_{k \vert k - 1} = {z}_{k - \vert k - 1} \qquad EQ . ( 3 )$$

$${σ}_{k \vert k - 1}^{2} = {σ}_{k - 1 \vert k - 1}^{2} + {σ}_{ϵ}^{2} \qquad EQ . ( 4 )$$

$${z}_{k \vert k} = {z}_{k \vert k - 1} + {σ}_{k \vert k - 1}^{2} [ {s}_{k} - \frac{1}{1 + {e}^{- ( α + {z}_{k \vert k} )}} ] \qquad EQ . ( 5 )$$

$${σ}_{k \vert k}^{2} = {[ \frac{1}{{σ}_{k \vert k - 1}^{2}} + \frac{{e}^{( α + {z}_{k \vert k} )}}{{[ 1 + {e}^{- ( α + {z}_{k \vert k} )} ]}^{2}} ]}^{- 1} \qquad EQ . ( 6 )$$

for k=1, 2, . . . , K. The initial conditions for the algorithm are z₀=z₀^{(l)} and σ_{0|0}²=σ_{ϵ}^{2(l)}. Since the term z_{k|k} appears on either side of (6) and the equation is non-linear, it can be solved using Newton's method.

Backward Filter: Based on the posterior mode estimate of z_{k|k} and the variance σₖ²_{|k}, a fixed-interval smoothing algorithm was used to compute z_{k|k} and σₖ²_{|k}. The fixed interval smoother is described as follows:

$${A}_{k} = \frac{{σ}_{k \vert k}^{2}}{{σ}_{k + 1 \vert k}^{2}} \qquad EQ . ( 7 )$$

$${z}_{k \vert K} = {z}_{k \vert k} + {A}_{k} ( {z}_{k + 1 \vert K} - {z}_{k + 1 \vert k} ) \qquad EQ . ( 8 )$$

$${σ}_{k \vert K}^{2} = {σ}_{k \vert k}^{2} + {A}_{k}^{2} ( {σ}_{k + 1 \vert K}^{2} - {σ}_{k + 1 \vert k}^{2} ) \qquad EQ . ( 9 )$$

for k=K−1, K−2, . . . , 1, where the initial conditions are z_{K|K} and σ_{K}²|K.

### Maximization Step

In the maximization step, the expected value of the complete data log likelihood is maximized according to the following:

$$EQ . ( 10 )$$

$${σ}_{ε}^{2 ( l + 1 )} = \frac{2}{K + 1} [ {\sum }_{k = 2}^{K} ( {σ}_{k \vert K}^{2} + {z}_{k \vert K}^{2} ) - {\sum }_{k = 2}^{K} ( A {σ}_{k \vert K} + {z}_{k \vert K} {z}_{k - 1 \vert K} ) ] + \frac{1}{K + 1} [ \frac{3}{2} ( {σ}_{1 \vert K}^{2} + {z}_{1 \vert K}^{2} ) - ( {σ}_{K \vert K}^{2} + {z}_{K \vert K}^{2} ) ]$$

$${z}_{ϵ}^{( l + 1 )} = \frac{1}{2} {z}_{1 \vert K} \qquad EQ . ( 11 )$$

The expectation maximization algorithm iterates between the E and M steps until the convergence of parameters. A change of variables formula is used on the Gaussian probability density function with mean z_{k|j} and variance σₖ²_{|j} to determine the probability density for qₖ, where j=k for the forward filter and j=K for the backward filter, as shown below (Smith et al. 2004):

$$EQ . ( 12 )$$

$$f ( q \vert α , {z}_{k \vert j} , {σ}_{k \vert j}^{2} ) = \frac{1}{\sqrt{2 π {σ}_{k \vert j}^{2}} q ( 1 - q )} \times exp \{ \frac{- 1}{2 {σ}_{k \vert j}^{2}} [ - {z}_{k \vert j} + log \frac{q}{( 1 - q ) {e}^{α}} ] \}$$

From (12), one can compute confidence intervals to determine, from an ideal observer's perspective, if a given peak occurred due to more than just random chance.

### Valence Index and Threshold Determination

During feature extraction and generation of the point process, thresholds are used to determine whether an observed feature is a significant deviation from a Gaussian distribution. These thresholds have to be selected for each subject to minimize the error between the predicted valence index and self-reported valence ratings of the subject throughout the respiration recording. Define the predicted valence index as p(xₖ>x_{median}), which provides a continuous value for the valence index in terms of the state's deviation from its median value. In other words, the predicted valence index represents the probability that the events are occurring due to more than just random probability (Wickramasuriya et al. 2020).

To compare the predicted valence index to the self-reported valence ratings, the self-reported valence ratings have to be converted into a continuous valence rating. First, for each subject, the valence ratings are normalized and rescaled on a scale of 0 to 1. The continuous valence rating is set equal to the value of the normalized valence rating at the intervals in which the corresponding video clip is shown. During periods between clips, the valence rating is linearly interpolated to create a continuous rating over time. Even though interpolation was performed, the true valence rating at these points is unknown, though the rating is expected to decrease in the absence of an emotional stimulus.

The first 25% of the respiration signal was considered for threshold determination and the remaining 75% of the signal was used to test the algorithm. To determine optimal threshold values, mean-squared error was calculated between the continuous self-reported valence rating and the predicted valence index. For each unique configuration of threshold values, a unique point process is generated, and valence state is estimated after the EM algorithm. The valence index and associated error was then calculated. Over all configurations of thresholds, the error was minimized to find the ideal thresholds that would cause the predicted valence index to best represent the self-reported valence rating. Using the thresholds found from the first portion of the signal, valence estimation is then performed for the entire signal.

### Model Accuracy

The accuracy of the valence estimation is determined by identifying whether the estimation properly identified periods of high and low valence in the subjects. This is done by categorizing each bin in the valence index and self-reported valence rating as either high valence, low valence, or neither. In the self-reported valence rating, valence was considered to be high if the index was above 0.8, and low if the index was below 0.2. These thresholds were selected empirically to represent periods of very high or very low valence throughout the duration of the recordings. For the valence index, valence was considered to be high if the index was above 0.9, and low if the index was below 0.1. These thresholds are selected because the index represents the probability of belonging in either state. Stricter definitions for high and low valence events were used to identify only the extremes of valence with high certainty, since only these periods would be significant enough to alert the user in the implementation of a control system for valence. Accuracy, sensitivity, and specificity are calculated based on whether each high valence period was properly identified by the estimation. For the purposes of accuracy calculations, the periods between clips are ignored. In addition, if the subject had a valence rating drastically conflicting with the expected valence response from a video (i.e. positive valence response to a horror movie clip) according to experimental setup, these portions are corrected to the expected response of the clip. These calculations are then repeated to determine if low valence periods were properly predicted in the valence index.

### Results

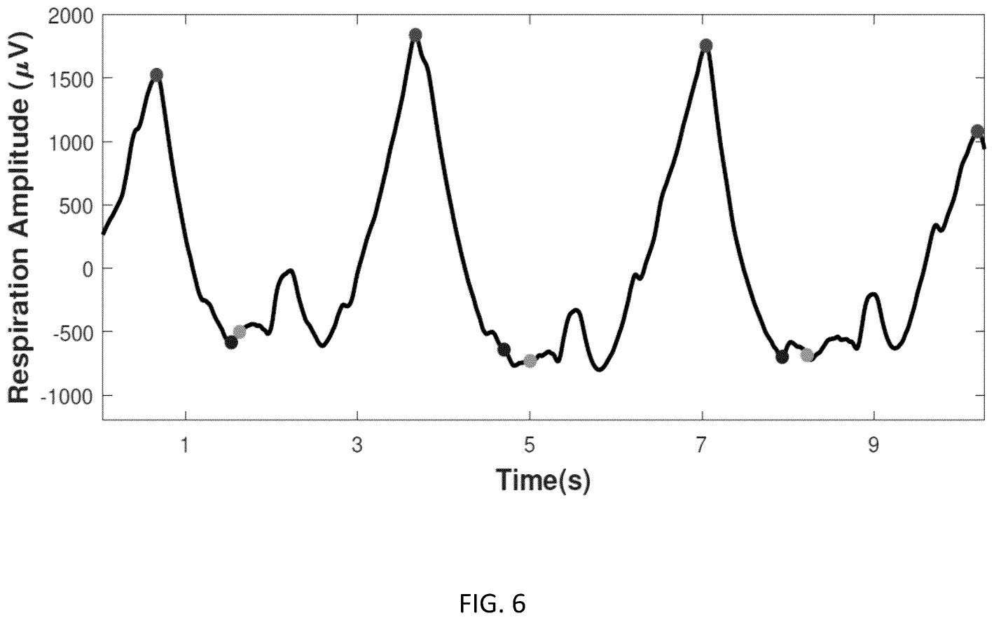

Sample results for peak detection, as well as detection of the beginning and end of breaths, are shown in FIG. 6. The cycle time is the time interval between the local minimum detected on either side of a respiration peak. For this subject, the respiration pattern contains an additional waveform prior to each breath. In this case, the start of inhalation is considered to be at the start of this smaller wave.

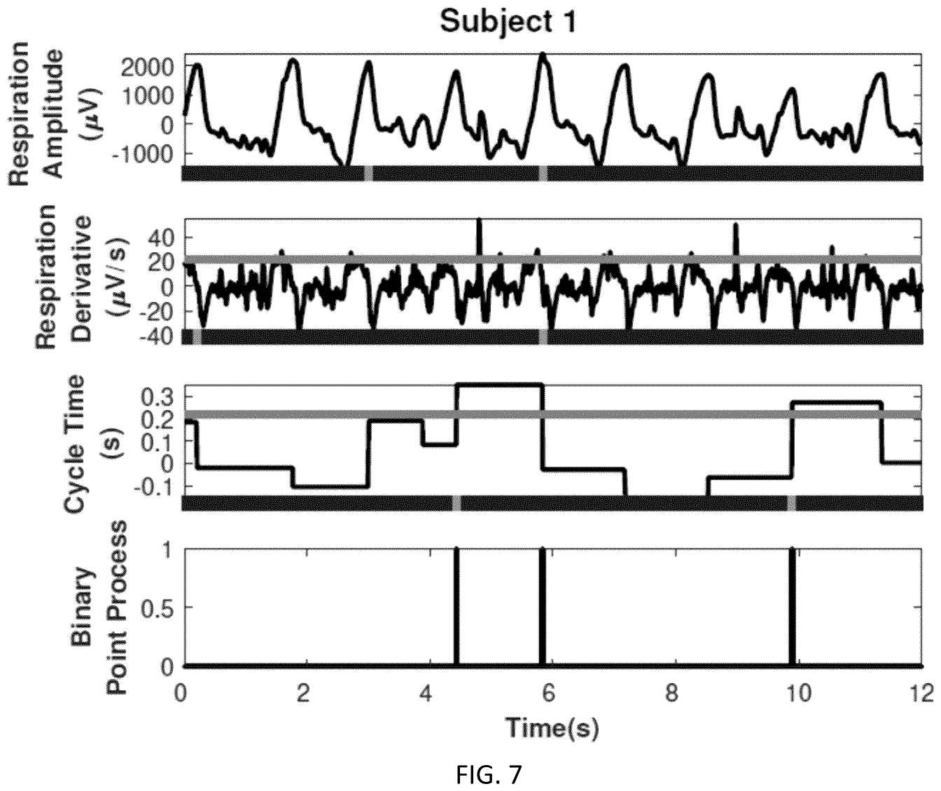

FIG. 7 shows a binary point process generated for a given respiration signal based on the three features. A separate binary point process is generated for each feature by marking the points at which the feature exceeds the threshold value. These point processes are combined to generate the final binary point process. The binary point process for each individual feature is also shown. For the respiration amplitude feature, the depth of each breath is measured based on the difference between the amplitude at the start and end of inhalation, rather than just the amplitude at the end of inhalation. For the respiration derivative feature, a point is generated when the derivative exceeds the threshold a certain number of times. This number limit is set to avoid extraneous derivative spikes not belonging to a breath that can be observed in the plot. The cycle time is shown as a continuous value of the change in cycle time corresponding to the most recent breath. A point is generated where the cycle time increases above the threshold. The resulting combined binary process contains the events that occurred in both the respiration amplitude and derivative features or only the cycle time feature.

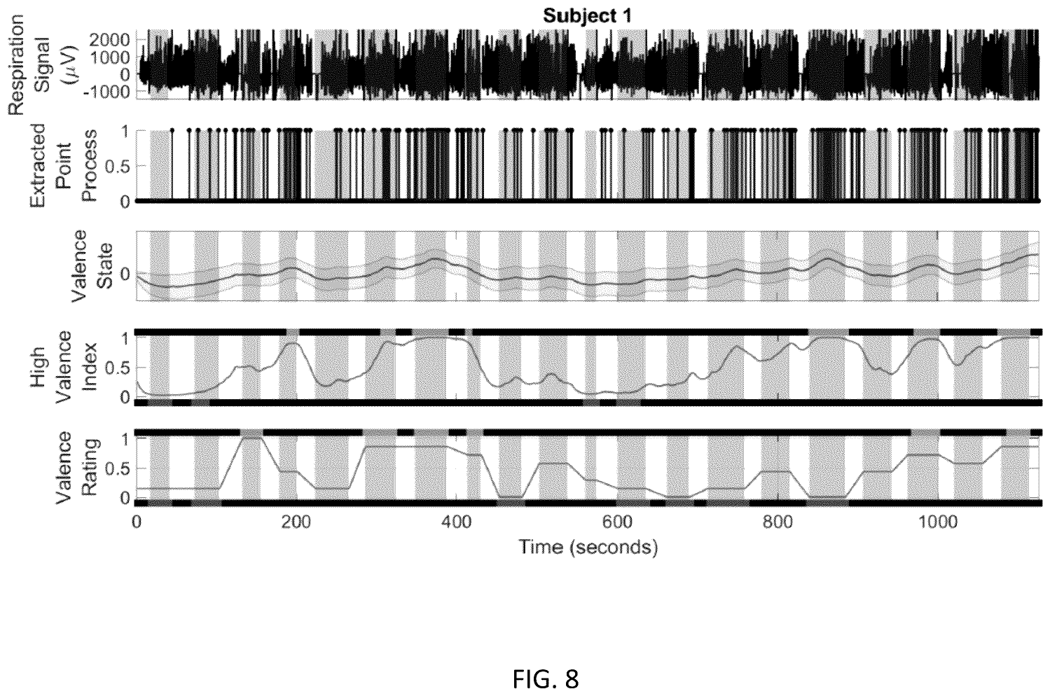

FIG. 8 shows the valence state estimation results for one subject. The pre-processed respiration signal, the binary point process, the estimated state, the estimated valence index, and the self-reported valence ratings are shown. Confidence intervals for state estimation are also shown in blue. The red intervals within each plot denote the intervals in which video clips are shown to the subject. The valence rating is converted into a continuous value for comparison to the predicted valence index. This is done by setting the continuous valence rating to the self-reported scores during clips and linearly interpolating for valence values between clips. This transformation allows us to directly compare the valence index to the self-reported scores.

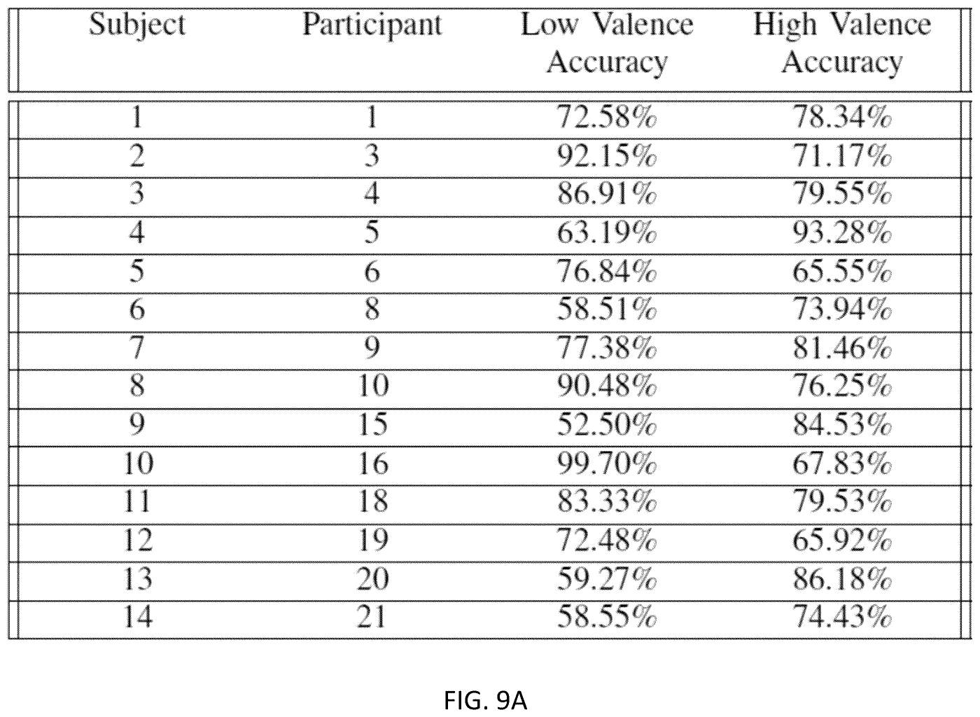

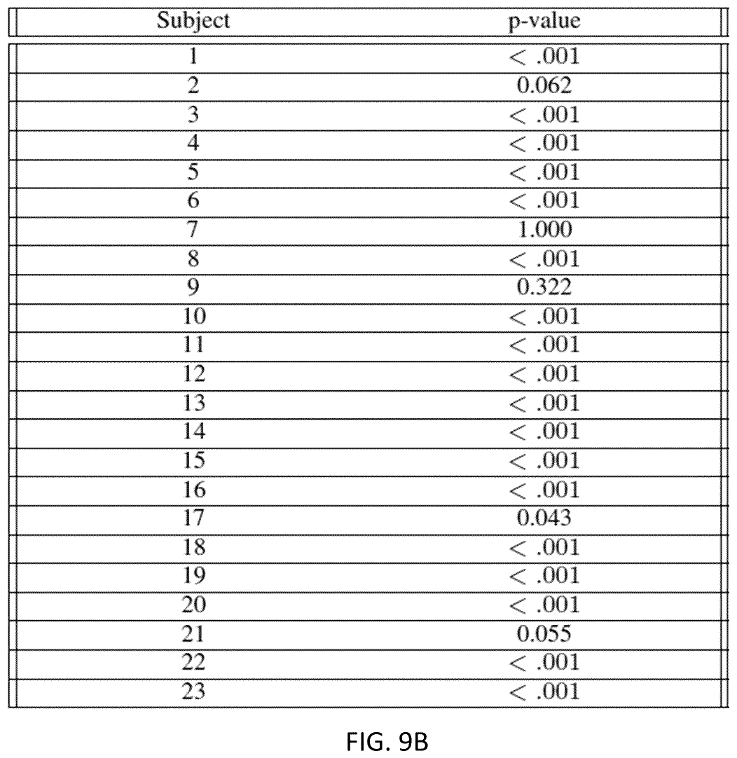

The accuracy of the model in predicting high valence and low valence events was found to be 77% and 73%, respectively. The sensitivity and specificity for high valence prediction was 0.47 and 0.85, while the sensitivity and specificity of low valence prediction was 0.21 and 0.92. The tables of FIGS. 9A-9B show additional results for the subjects of the experiments.

### Discussion and Conclusions

Respiration peak amplitude, rate of respiration, and respiration cycle time appear to be indicators of high valence. For most subjects, during high valence periods, there would be a very high density of pulses in the binary point process as shown in FIG. 8. In some cases, there is also a high density of pulses found in the intervals between video clips being shown. This observation could be a result of the subject's valence being unreported during these intervals, and the subject could have had an emotional response during this resting period. It was also observed that the respiration signal is more prone to motion artifacts between clips that could be attributed to this observation.

The valence index calculated based on the latent valence state appears to correlate to the self-reported valence ratings provided by most subjects. However, the quality of correlation varies greatly among different subjects. In more strongly correlated subjects, the predicted valence can capture faster changes in valence (i.e. a brief period of high valence between extended periods of low valence) and even depicts a drop in valence between clips being visible as shown in the estimation for Subject 1 in FIG. 8. In moderately correlated subjects, the predicted valence follows general trends in valence (extended periods of high or low valence) but fails to capture faster changes in valence. In weakly correlated subjects, the predicted valence appears to be slow changing and typically within the neutral valence range. This could be a result of these subjects providing a large number of neutral valence ratings, or the features not being good indicators of valence in these specific subjects. In all subjects, the predicted valence significantly deviates from the reported valence during fast changes in valence in some clips. During these fast changes, the incorrect estimate continues to follow the general trend in valence rather than an abrupt change.

The accuracy of the model in predicting high valence periods validates the potential applications of the model in monitoring the valence state. The high specificity values indicate that when the valence index is above 0.9, the subject is very likely to be experiencing high valence. In comparison, the low valence event prediction yielded lower accuracy values. However, this could be a result of the valence index reaching the lower threshold less frequently. This model seeks to predict high valence from increases in depth of breath and rate of respiration. These features could be less effective in determining the lower end of the valence spectrum since reductions in respiration amplitude were not measured and that could be more indicative of low valence. The model also has a significantly lower sensitivity than specificity. This could be a result of stricter thresholds for high and low valence period determination in the valence index. These thresholds were stricter to only identify the extreme valence periods. However, the self-reported ratings contained longer and more frequent high and low valence periods than the index, resulting in a high specificity but low sensitivity. These periods could also be affected by the subject's interpretation of the valence rating or individual bias. It should also be noted that the self-reported rating could be inaccurate in representing the subject's true valence level since the ratings are interpolated from only 20 ratings.

The accuracy of the predicted valence also depends on the accuracy of the self-reported valence ratings. Self-reported ratings could be subject to bias and have high subject variability (Eaton et al. 2001). In some cases, subjects would provide only high or low valence ratings but fail to provide neutral ratings. In other cases, subjects provided mostly neutral ratings with few ratings in either extreme. Normalizing the self-reported ratings can correct for this issue in some subjects, but a true accuracy of the predicted valence cannot be determined without an objective measure of valence.

From a physiological perspective, respiration peak amplitude, rate of respiration, and respiration cycle time appear to be indicators of high valence. High valence is associated with deep and faster breathing (Sato et al. 2020) (Zhang et al. 2017). During deep breaths, the respiration amplitude would increase as the abdomen expands further due to the increase in air volume. During faster breathing, the rate of inhalation and exhalation would increase to compensate for the increased amplitude. This physiological response to an emotional stimulus is captured in the features as deviations from the typical physiological behavior. It should also be noted that these features could only be indicative of high valence, which would explain the decrease in quality of estimation as the subjects' valence rating fluctuates.

The accuracy of the model makes it ideal for continuous monitoring of valence over extended periods. In a practical application to continuously estimate valence, the valence parameter estimation would have to be performed in batches of about 3-minute intervals. Threshold estimation would have to be performed for one batch per subject given that self-reported valence levels are also provided. This model can be improved by implementing a more complex filter that incorporates the amplitude of impulses. The amplitude values can also contain information that would make the estimate more robust to minor changes in valence. The model can be further improved by incorporating other physiological signals that have shown to be predictors of emotion to improve the quality of prediction. The model can also include incorporating it with previous work performed with predicting arousal as in Wickramasuriya (Wickramasuriya et al. 2020) to estimate a two-dimensional emotion state including both valence and arousal.

Further details of the system and additional experimental results are described below. Changes in respiratory activity are one of the most apparent physiological responses to emotion (Vleminca et al. 2015). In the absence of environmental stimuli, respiration is predominantly controlled by the dorsal and ventrolateral respiratory groups located in the pons and medulla of the brain (Tortora et al. 2018). The dorsal group initiates inhalation depending on sensory input, which is typically the concentration of carbon dioxide in the bloodstream. The ventrolateral group initiates exhalation depending on mechanical changes in the lungs (i.e. increasing airway pressure) (Mitchell et al. 1975). Though the relationship between amygdala activity and these respiratory regions are poorly understood, the emotional response defined by the amygdala has been shown to directly alter the pattern of respiration (Homma et al. 2008). For example, fear and anxiety have been shown to induce shallow and rapid breathing, circumventing the typical sensory input to the dorsal respiratory group (Eaton et al. 2001) (Wilhelm et al. 2001) (Egger et al. 2019). This behavior, in addition to recent developments in wearable sensors allowing for the continuous monitoring of respiration (De Fazio et al. 2021), makes respiratory activity ideal for tracking changes in emotion.

The ability to continuously track changes in emotion has a wide range of applications including remote monitoring of patients with mental disorders such as depression, improving human-computer interaction, and even multimedia recommendation. Respiration is also unique from other physiological signals commonly used to track emotional changes, such as galvanic skin response (GSR), electroencephalogram (EEG) activity, and electrocardiogram (ECG) activity, in that it can be manually controlled (Eaton et al. 2001). It has even been shown that manual correction to respiratory activity following negative emotions can alleviate detrimental mental and physical responses to a stimulus (Payne et al. 2003) (Whalen et al. 1998), enabling the implementation of a control loop to regulate an emotional response using the same respiration signal.

Emotion recognition experiments typically measure physiological signals as participants are exposed to various emotional stimuli, typically visual or auditory stimuli (Phelps 2004) (Vlemincx et al. 2015). Previous studies estimating valence using physiological signals have successfully used machine learning to classify signals based on the emotional response. This classification involves categorizing emotional responses into either specific emotions (amusement, sadness, fear etc.) or level of valence (low, neutral, or high). The highest classification accuracies were typically obtained using EEG signals relative to other physiological signals, since an EEG can directly measure brain activity driving an emotional response (Phelps 2004) (Bazgir et al. 2018) (Balasubramanian et al. 2018) (Liu et al. 2019). However, EEG devices are prone to artifacting and noise and uncomfortable for long-term use (Roy et al. 2021), making the EEG signal impractical for continuous valence monitoring applications. On the other hand, using a combination of peripheral physiological signals such as ECG, GSR and respiration yields lower classification accuracies (Dominquez-Jimenez et al. 2020) but are more practical to measure for continuous monitoring. Deep learning methods have also been successfully used on only respiration signals to classify breathing patterns as high or low valence (Zhang et al. 2017). These previous studies successfully identify categorical changes in valence present in physiological signals but do not quantify changes in valence with a continuous numerical value. In addition, machine learning and deep learning approaches do not reveal information on the underlying physiology of the emotional response.

A continuous numerical estimate of valence is particularly useful to observe trends in valence to identify periods of high and low valence, as well as quantifying the degree of high or low valence experienced. For example, someone experiencing a period of joy and amusement can better distinguished with a numerical estimate of valence rather than a categorical estimate. State-space models have been previously used to estimate several unobserved neurological processes using physiological signal information as well as behavioral data. In this study, a state-space model was developed for tracking valence based on changes in respiratory activity by generating a marked point process (MPP) similar to (Wickramasuriya et al. 2020) for inferring valence events corresponding to an emotional response based on changes in respiratory pattern. The resulting MPP contains information regarding both the temporal location and magnitude of the inferred valence event. The state-space model relates the inferred valence events to an unobserved valence state based on features derived from a respiration signal collected via a respiration belt. A separate MPP is created for valence events that correspond to high valence (positive emotion) and low valence (negative emotion). Subsequently, the high valence index (HVI) and low valence index (LVI) are estimated using an Expectation-Maximization (EM) algorithm. These indices are then combined to provide an improved estimate of valence. The estimated valence is then compared to self-reported ratings of valence throughout the recording.

### Data

To assess the behavior of respiratory activity in response to emotional changes in valence, continuous recordings were required for respiratory activity in the presence of various emotional stimuli. The Multimodal Database for Affect Recognition and Implicit Tagging (MAHNOB-HCI) is a publicly available database that contains an emotion recognition experiment in which various physiological signals, including respiratory activity, are measured as participants are shown video clips meant to elicit emotional responses (Soleymani et al. 2012). The database comprises recordings taken from 27 participants. Over an approximately 50-minute period, participants are shown 20 video clips meant to elicit different emotional responses. Each participant is shown the same video clips in a randomized order. For each trial in which a video clip is shown, the participant is shown a video meant to elicit no emotion, a video meant to elicit a specific emotion (fear, joy, anxiety etc.) depending on the content of the video, and then self-assess their emotional response in terms of valence along with other emotional measures on a scale of 1 to 9. Participants are previously informed on the definitions of the various emotional measures such as valence and arousal as well as how to quantify their emotions using the numerical scale using a self-assessment manikin.

Throughout the approximately 50-minute recording period, various physiological signals including galvanic skin response, ECG, respiration, skin temperature, and EEG were measured. Respiratory activity was measured via a respiration belt around the chest of the participant to measure expansion and contraction of the lungs as the participant breathes. From the 27 total participants, several participants were removed due to nonconsenting for publication, technical difficulties, and significant respiratory measurement noise, leaving 17 participants for analysis.

### Pre-Processing

A typical respiration signal collected via respiration belt could be affected by measurement noise, motion artifacting, and baseline drift that is typically found in long term respiration activity recordings (Liu et al. 2019). To correct the baseline and remove unwanted high and low frequency measurement noise, a Butterworth bandpass filter was implemented with cutoff frequencies of 0.05 Hz and 1 Hz. The filter corrects any polynomial baseline trends to zero and removes unwanted high frequency noise present in the signal. These frequencies are selected such that the components of respiratory activity are preserved but high frequency noise and low frequency oscillations in the baseline of the signal are removed (Sato et al. 2020). Potential motion artifacts are observable in the respiration activity recordings as rapid and significant changes in the baseline and spiking activity of the signal. These artifacted regions contain peaks that are 5-10 times the amplitude of typical breath in the same participant and affect the measurement of subsequent breaths. To remove these regions from the analysis, a moving average filter was implemented on the respiration signal to identify regions where the significant change in baseline and peak amplitude is not physiologically possible. These regions are then removed before analysis is performed on the respiration signal.

### Feature Extraction

Evidence of an emotional response can be observed in several features in a respiration signal. Prior studies have found that both the depth of breath and rate of breathing relate to changes in valence (Wilhelm et al. 2001), (Zhang et al. 2017), (Bruce 1996). These components can be retrieved from the respiration signal as the amplitude of the respiration signal following inhalation and the derivative of the respiration signal during inhalation and exhalation. The derivative of the respiration signal is particularly important because it can be used to quantify the speed of inhalation, which is directly modulated by neuronal input corresponding to an emotional response (Homma et al. 2008). This neuronal input is typically driven by increases in carbon dioxide concentration of the blood that signal the lungs to initiate inhalation. By assuming the carbon dioxide levels are consistent across each breath and the participant is not manually controlling their respiratory activity, one can model the rate of inhalation as a Gaussian random variable in the absence of confounding factors, such as emotion, environment, or even illness (Vlemincx et al. 2015), (Bruce 1996). In other words, the depth of each breath and rate of inhalation should be consistent across each breath with minuscule error between breaths caused by unobserved peripheral biological processes.

The Gaussian nature of typical respiratory activity (Bruce 1996) can be exploited to detect changes in these respiration features that may correspond to neuronal input from an emotional response. In terms of valence, faster and deeper respiration has been shown to occur in response to positive emotions, whereas shallow respiration typically occurs in response to negative emotions (Zhang et al. 2017), (Jerath et al. 2020). To extract the features of interest, the location of breaths was first detected using the MATLAB findpeaks function. The function detects the local maxima throughout the respiration signal that corresponds to the end of inhalation for each individual breath. A window size of one second was used for peak detection, meaning that two breaths cannot occur within one second. This window size is specific for this experiment and should be adjusted according to the application. For example, in the case of monitoring mental disorders such as post-traumatic stress disorder, breaths could be occurring much more rapidly relative to video clip viewing and the window size would have to be adjusted accordingly (Tol et al. 2013). The findpeaks function is also used on the inverted respiration signal to detect the local minima surrounding each local maximum that correspond to the start of inhalation and end of exhalation. To extract information regarding the rate of inhalation that corresponds to fast or slow breathing, the mean of the derivative of the respiration signal between the start of inhalation and the end of inhalation was calculated. This value contains information regarding the speed of inhalation and, also, correlates to the depth of the breath, since faster breathing would typically occur in tandem with deep breaths in a positive valence response. To extract information regarding the depth of each breath, the difference between the amplitude of the respiration signal at the end of inhalation and the start of inhalation was measured. This value corresponds to the total expansion of the lungs that correspond to each breath. Based on these extracted features, the rate of inhalation and amplitude of each breath, one is able to quantify the speed and depth of each breath, which will be used to identify the location of potential valence events related to neural activity corresponding to a change in valence.

### Marked Point Process Generation

The marked point process describes both the location and magnitude of the inferred valence events relating to an emotional response. Using the measured feature values at each breath, one can identify potential valence events by comparing the features associated with each breath to the expected values according to the Gaussian distribution. The parameters for the Gaussian distribution, the mean and standard deviation, are subject-specific and calculated for each feature based on the previous history of breaths. In this case, the mean and standard deviation was calculated for both the rate of inhalation and depth of breath across all breaths in the respiration signal. For every breath, the probability of each feature occurring was calculated according to the Gaussian probability density function. These probabilities are used to identify potential valence events that correspond to high and low valence in two separate marked point processes based on one of the features.

For the generation of the high valence MPP, it was found that the rate of inhalation is more informative since it contains information regarding increases in the speed of inhalation and correlates to increases in the amplitude of the breath (Zhang et al. 2017). Subject-specific probability thresholds were set to detect potential valence events for each breath. The thresholds are unique for each feature and have been determined empirically. They are required to be unique since the impact of an emotional response on respiration can vary drastically between participants. In other words, while two people may both be experiencing high valence, the magnitude of change in their breathing activity is dependent on their susceptibility to an emotional response. Using these empirically found thresholds, a point process is generated based on the probability values associated with each breath being below the probability threshold. Define the magnitude of each point as the inhalation rate corresponding to the breath. These magnitudes are then rescaled between 0 and 1. The result is a marked point process containing the locations of inferred valence events, based on the probability of each feature occurring, and a magnitude, based on the increasing rate of inhalation.

In one experiment, for the generation of the low valence MPP, it was found that the amplitude of each breath is more informative than the rate of inhalation. This is likely due to the total length of a respiratory cycle shortening during low valence and high arousal (Zhang et al. 2017), (Jerath et al. 2020), which would result in the rate of inhalation being less impacted or unchanged. However, the respiration signal would still show decreased amplitudes at the end of inhalation. Using the probability values calculated according to the Gaussian probability distribution for each breath with decreased peak amplitude, the location of potential valence events was defined similar to the high valence MPP. The magnitude of each stimulus is defined as the difference between the mean depth of breath and the depth of breath of that particular breath. These values are then rescaled between 0 and 1 similar to the high valence MPP. The resulting marked point process contains information regarding the events associated with decreases in depth of breath that likely correspond to low valence.

In another experiment, for the generation of the high and low valence MPP, each breath was labeled as indicative of neutral, high or low valence using a k-means clustering algorithm. The k-means algorithm is able to create subject-specific valence event identification criteria without the need for training on self-reported ratings or expert labels. In some embodiments, the k-means algorithm is unsupervised. The algorithm also accounts for potential variability of a valence response in participant. In other words, while two people may both be experiencing high valence, the magnitude of change in their breathing activity is dependent on their susceptibility to an emotional response. As a result, using uniform thresholds for all participants would be ineffective in estimation efforts. Using the known locations of the start of inhalation, end of inhalation, and end of exhalation, breath amplitude, inhalation time, and inhalation rate are measured for each breath. Labeling is initially performed with two clusters, in which the cluster with the smaller centroid values is assigned to low valence. All breaths with features containing an absolute value of less than 0.2 were removed from the labeling process. These breaths drastically increase the error in accuracy for the k-means algorithm and likely correspond to non-emotion related breaths. To assign a magnitude to each valence event, the mean absolute difference between the breath amplitude for both the high and low valence MPP was taken. The MPPs are then rescaled from 0 to 1 before state estimation is performed.

To avoid convergence of the k-means algorithm to local minima or overfitting to one of labels, the labeling of breaths and MPP generation are repeated over 10 iterations. In cases where the k-means algorithm overfits to one label (typically low valence due to its closer proximity to neutral valence breaths), the number of clusters is increased until overfitting is prevented. In these cases, the cluster with the smallest centroid values is classified as low valence. For each iteration, a moving average filter is applied to both MPPs. The averaged high and low MPPs which display the strongest negative correlation are then used for valence estimation. This is because high and low valence events are unlikely to coincide unless the individual's is manually manipulating their own breath, or the breaths are likely not indicative of valence. In addition, if either marked point process does not contain a feasible number of events by setting minimum and maximum thresholds for the number of high and low valence events the algorithm is reiterated.

### K-Means Clustering Algorithm

A k-means clustering algorithm is used to group individual breaths into either high or low valence events based on features extracted from a respiration belt. These features include the amplitude of the breath, the inhalation time, the inhalation rate, and interaction terms between the features. Initially, breaths are grouped with two clusters, but the number of clusters is later adjusted to prevent overfitting. A cityblock distance metric is used for minimization. Specifically, this method is used to reduce the impact of outliers on the clustering of high valence indicative breaths by considering the median value of features for cluster centroids. The MATLAB implementation of the k-means++ algorithm is used for cluster center initialization with a maximum of 30 iterations allowed for convergence of cluster centroids. During clustering, breaths that pertain to neutral valence or noise have to be removed from analysis. This is done by taking the z-score of all breath amplitudes and removing breaths less than 0.2 or greater than 5. The cluster centroid with a smaller L2 norm is assigned as low valence indicative, whereas all other breaths are considered to be high valence indicative.

The k-means clustering algorithm is prone to overfitting when there is no clear separation between features. Though the respiration patterns that correspond to very high and very low valence may be unique, the presence of a neutral valence respiratory pattern complicates this separation. Though the removal of normal breaths greatly reduces the impact of neutral valence breaths, the clustering is still susceptible to overfitting to low valence and converging to local minima. To prevent poor clustering and obtain optimal results, the k-means algorithm is iterated over 10 iterations. Several steps are taken to prevent the overfitting to one of the labels as the eventual non-convergence of the MPP filter. Heuristic upper and lower limits are set on the number of events for the low or high valence MPP based on experimental conditions. The upper limit is set to the signal duration, and the lower limit is set to 0.02× signal duration, where the signal duration is given in seconds. Both limits are based on experimental feasibility and may need to be adjusted for different scenarios. For example, the lower limit for pulses assumes pulses have to occur, but, in reality, a person may never change from neutral valence. In this particular setup, a certain minimum number of valence indicative events was expected due to the viewing of emotion eliciting clips. The upper limit may not hold in more extreme changes in emotion, such as in the case of posttraumatic stress disorder or panic disorder. In these cases, the respiration rate may be drastically impacted by the underlying condition leading to the feasibility assumptions to fail.

In addition to the prevention of overfitting, an additional parameter is measured to ensure the optimal labeling of breaths has been achieved. To ensure that high and low valence MPPs contain unique information, the negative correlation between the high and low valence MPPs was calculated. To do this, a moving average filter is applied after the generation of the MPPs based on k-means labeling. The resulting signals contain information on the averaged amplitude of valence events over time. The Pearson correlation coefficient is calculated between the moving-averaged high and low valence MPPs and select the iteration which maximizes the negative correlation between the two signals. The negative correlation is maximized to reduce the coincidence of high and low valence events, since a person is unlikely to experience high and low valence simultaneously or in very short intervals.

### State-Space Model

The state-space model relates the probability of the valence events occurring to an unobserved valence state. A separate valence state is calculated using the high valence MPP and low valence MPP. A high valence state is calculated based on the inferred valence events associated with increasing inhalation rate that is associated with pleasant emotion. The low valence state is calculated based on inferred valence events associated with decreases in depth of breath commonly associated with low valence. For both states, a state-space model previously used for tracking sympathetic arousal is used (Dzedzikis et al. 2020), (Egger et al. 2019) to track valence. Similar to previous models, the state-space model assumes the latent valence state for both high and low valence follows a random walk as:

$${x}_{j} = {x}_{j - 1} + {ϵ}_{j} \qquad EQ . ( 13 )$$

where ϵⱼ˜N(0, σ_{ϵ}²) is the process noise. The presence of valence events nⱼ in the marked point process are modeled as a Bernoulli distributed random variable with probability mass function pⱼⁿ^{ⱼ}(1−pⱼ)¹⁻ⁿ^{ⱼ} where pⱼ=P(nⱼ=1). One can relate the state and probability of a valence event occurring using a logit transform as shown.

$$log ( \frac{{p}_{j}}{1 - {p}_{j}} ) = β + {x}_{j} ⇒ {p}_{j} = \frac{1}{1 + {e}^{- ( β + {x}_{j} )}} \qquad EQ . ( 14 )$$

According to this equation, the probability of observing valence events associated with either valence state increases as the respective valence state increases. For example, during high valence, the occurrence of valence events associated with the increasing inhalation rates would be higher during increases in valence. β is a parameter that is to be determined empirically assuming the state xⱼ begins at zero at the start according to the equation:

$$β \approx log ( \frac{{p}_{0}}{1 - {p}_{0}} ) \qquad EQ . ( 15 )$$

One can approximate p₀ as the mean probability of a valence event occurring over the duration of the recording. This number is unique for each participant and represents the physiological baseline for the valence event probability. In reality, this probability would depend on the specific emotional stimuli a person regularly experiences throughout the day, which would be highly subject-specific and could vary significantly over time for one person.

Next, consider the amplitude of each inferred valence event rⱼ assuming that the degree of change in high or low valence corresponds to the degree of change in the respective feature. In the case of the high valence state, the participants' change in inhalation rate would increase as they experience increasing levels of valence. Similarly, for the low valence state, the depth of breath would likely decrease as they experience decreasing levels of valence. Previous models describing neural states have used a linear or log-linear relationship between the state variable and continuous values. Using a linear model relating the valence event amplitudes and the valence state similar to (Coleman et al. 2011) (Prerau et al. 2009), define the relationship as:

$${r}_{j} = {γ}_{0} + {γ}_{1} {x}_{j} + {v}_{j} \qquad EQ . ( 16 )$$

where vⱼ˜N (0, σᵥ²) represents sensor noise and model error. γ₁ and γ₀ are coefficients relating the valence state to the amplitude of the inferred valence events that are to be determined. The joint probability for observing a valence event given an amplitude values can be written as:

$$p ( {n}_{j} ⋂ {r}_{j} \vert {x}_{j} ) = \{ \begin{matrix}1 - {p}_{j} & if {n}_{j} = 0 \\ {p}_{j} \frac{1}{\sqrt{2 π {σ}_{v}^{2}}} {e}^{\frac{- {( {r}_{j} - {γ}_{0} - {γ}_{1} {x}_{j} )}^{2}}{2 {σ}_{v}^{2}}} & if {n}_{j} = 1\end{matrix} \qquad EQ . ( 17 )$$

According to the equations, an amplitude is only present when a valence event is present and is modeled as a Gaussian distributed variable. The valence state xⱼ and the parameters of the model γ₀, γ₁, σᵥ², σ_{ϵ}² are to be determined. The high valence state and low valence state and their respective model parameters is estimated using an Expectation-Maximization framework similar to (Mendel 1995) and (Brown et al. 1998). The algorithm will iterate between expectation and maximization until convergence of parameters.

### Expectation Step

Given the marked point processes containing the presence of valence events and their associated amplitudes Y^{J}=(n₁, r₁), (n₂,r₂), . . . (nⱼ,rⱼ), respective valence states xⱼ are predicted in the expectation step. A forward and backward Bayesian filtering approach is used to obtain a Gaussian approximation of the posterior probability p (xⱼ|yⁱ) at each time bin. The prediction and update equations in the forward filter are as follows:

### Predict:

$${x}_{j \vert j - 1} = {x}_{j - 1 \vert j - 1} \qquad EQ . ( 18 )$$

$${σ}_{j \vert j - 1}^{2} = {σ}_{j - 1 \vert j - 1}^{2} + {σ}_{ϵ}^{2} \qquad EQ . ( 19 )$$

### Update:

If nⱼ=0

$${x}_{j \vert j} = {x}_{j \vert j - 1} + {σ}_{j \vert j - 1}^{2} ( {n}_{j} - {p}_{j \vert j} ) \qquad EQ . ( 20 )$$

$${σ}_{j \vert j}^{2} = {[ \frac{1}{{σ}_{j \vert j - 1}^{2}} + {p}_{j \vert j} ( 1 - {p}_{j \vert j} ) ]}^{- 1} \qquad EQ . ( 21 )$$

If nⱼ=1

$${C}_{j} = \frac{{σ}_{j \vert j - 1}^{2}}{{γ}_{1}^{2} {σ}_{j \vert j - 1}^{2} + {σ}_{v}^{2}} \qquad EQ . ( 22 )$$

$${x}_{j \vert j} = {x}_{j \vert j - 1} + {C}_{j} [ {σ}_{v}^{2} ( {n}_{j} - {p}_{j \vert j} ) + {γ}_{1} ( {r}_{j} - {γ}_{0} - {γ}_{1} {x}_{j \vert j - 1} ) ] \qquad EQ . ( 23 )$$

$${σ}_{j \vert j}^{2} = {[ \frac{1}{{σ}_{j \vert j - 1}^{2}} + {p}_{j \vert j} ( 1 - {p}_{j \vert j} ) + \frac{{γ}_{1}^{2}}{{σ}_{v}^{2}} ]}^{- 1} \qquad EQ . ( 24 )$$

The filter equations are a combination between (Smith et al. 2004) and (Prerau et al. 2009) that include both the binary observation behavior in the absence of a valence events and consideration of the continuous parameters relating to the amplitude of the valence events. Since x_{j|j} appears on both sides of equations (20) and (23), one can use the Newton-Raphson method to approximate the state variables. Once the forward filtering is performed, the estimated state is improved by reversing the direction of the signal and performing backwards filtering as follows:

$${A}_{j} = \frac{{σ}_{j \vert j}^{2}}{{σ}_{j + 1 \vert j}^{2}} \qquad EQ . ( 25 )$$

$${x}_{j \vert J} = {x}_{j \vert j} + {A}_{j} ( {x}_{j + 1 \vert J} - {x}_{j + 1 \vert j} ) \qquad EQ . ( 26 )$$

$${σ}_{j \vert J}^{2} = {σ}_{j \vert j}^{2} + {A}_{j}^{2} ( {σ}_{j + 1 \vert J}^{2} - {σ}_{j + 1 \vert j}^{2} ) \qquad EQ . ( 27 )$$

### Maximization Step

From the estimated state values obtained from the expectation step, one may wish to estimate the model parameters by maximizing the data likelihood. By using the following equations similar to (Wickramasuriya et al. 2020) and (Dempster et al. 1977) one can estimate the expectation of the log likelihood and calculate the model parameter values:

$$EQ . ( 28 )$$

$${\sum }_{j = 1}^{J} 𝔼 [ {n}_{j} ( β + {x}_{j} ) - log ( 1 + {e}^{β + {x}_{j}} ) ] + \frac{( - \vert \tilde{J} \vert )}{2} log ( 2 π {σ}_{v}^{2} ) - {\sum }_{j ϵ J} \frac{𝔼 [ {( {r}_{j} - {γ}_{0} - {γ}_{1} {x}_{j} )}^{2} ]}{2 {σ}_{v}^{2}} + \frac{( - \vert \tilde{J} \vert )}{2} log ( 2 π {σ}_{ε}^{2} ) - {\sum }_{j ϵ J} \frac{𝔼 [ {( {x}_{j} - {x}_{j - 1} )}^{2} ]}{2 {σ}_{ϵ}^{2}}$$

where J is the total possible event locations and \tilde{J}={j|nⱼ=1}. The algorithm iterates between estimating the valence states in the expectation step and improving the parameter estimations in the maximization step until convergence of parameters. The model parameters were considered to have converged once the mean absolute is below a toleration threshold of 10⁻⁶.

### Combined Valence Index Determination

From the estimated state variables obtained from the Expectation-Maximization algorithm, one can calculate an index for high and low valence states separately based on the behavior of the states. Define the index of each respective state as p(xⱼ>x_{median}), which defines the index as a continuous value which represents the state's deviation from the median value. This value indicates the probability that valence events are occurring due to more than chance and represents the ideal observer certainty level. The high and low valence index calculated from either MPP are then combined according to the following equation:

$$Combined Valence Index = \frac{1}{2} + \frac{1}{2} HVI - \frac{1}{2} LVI \qquad EQ . ( 29 )$$

The resulting combined valence index, ranging from 0 to 1, considers both the valence events representative of low valence changes and the valence events representative of high valence changes to define a more accurate estimated valence index based on deviation from a neutral valence of 0.5. The high valence index is added to consider pleasant changes in valence from the baseline neutral valence, and the low valence index is subtracted to denote a high occurrence of unpleasant changes in respiration. In the resulting combined index, a value of 1 indicates very high valence, 0 indicates very low valence, and 0.5 represents a neutral emotion, which is indicated by the absence of high or low valence events.

To compare the valence indices to the self-reported ratings of the participants, the self-reported ratings were converted into a continuous variable. 20 ratings are provided throughout the duration of the respiration recording corresponding to each of the 20 video trials. The continuous value for valence rating is set to be equal to the provided rating during the corresponding trial and linearly interpolated between trials where the valence levels are unknown. This is done to observe general trends in valence, but, in reality, the participant's instantaneous valence level is likely to decrease between trials in the absence of an emotional stimulus. The continuous values of the self-reported rating are then normalized and rescaled between 0 and 1 to compare directly to the calculated combined valence index.

Previously, empirically determined probability thresholds were used to define the presence of valence events. These thresholds were determined by minimizing the mean-squared error between the combined valence index and the self-reported valence rating. The first 25% of the respiration signal was used to determine these threshold values. In the case that the first 25% of the signal does not contain significant changes in reported valence, the first 25% of the signal that contains both a high valence and low valence component was used. The purpose of this is to provide a measure of the participant's susceptibility to an emotional response, which is unique to each participant and difficult to estimate without the knowledge of a ground truth.

### Model Accuracy

Model accuracy was determined by identifying high and low valence periods in the self-reported ratings. Periods were determined by setting thresholds on the normalized ratings. High and low valence periods were identified as periods where the ratings were above 0.75 or below 0.25, respectively. The combined valence index is used to determine estimated locations of high and low valence in the participant. Since the index can be interpreted as the probability of belonging in either a high or low valence state, high and low valence periods were identified as being above 0.8 or below 0.2, respectively. These thresholds are different from the self-reported rating threshold since the goal was to indicate, with high certainty, the presence of a high or low valence event. Sensitivity, specificity, and accuracy are calculated separately for high and low valence based on whether the periods are properly identified during the trials. High or low valence periods detected between trials are removed from consideration. In addition, trials were removed in which the participant had a conflicting response to the stimuli. For example, if a participant responds to a video clip meant to elicit disgust with amusement, the natural respiratory response to the emotion may have been impacted by manual modulation.

### Statistical Analysis

Statistical analysis was performed by comparing the qualities of the inferred valence events and combined valence estimations for high and low periods. High and low periods are identified based on self-reported ratings. Self-reported ratings are normalized and re-scaled between zero and one. High valence periods are considered to be greater than 0.5 and low valence periods are considered to be less than 0.5. For each participant, the numerical valence estimation based on the combined valence index is compared over all high and low valence periods using a right-tailed Wilcoxon rank sum test. In addition, the number of valence events, ∥n∥₁, and mean amplitude of valence events are measured in either period and compared.

### Results

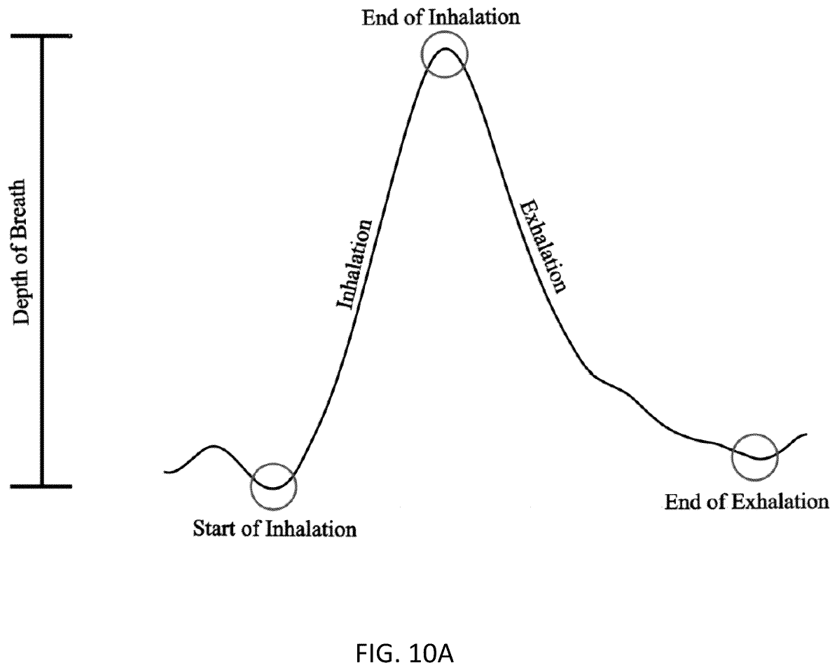

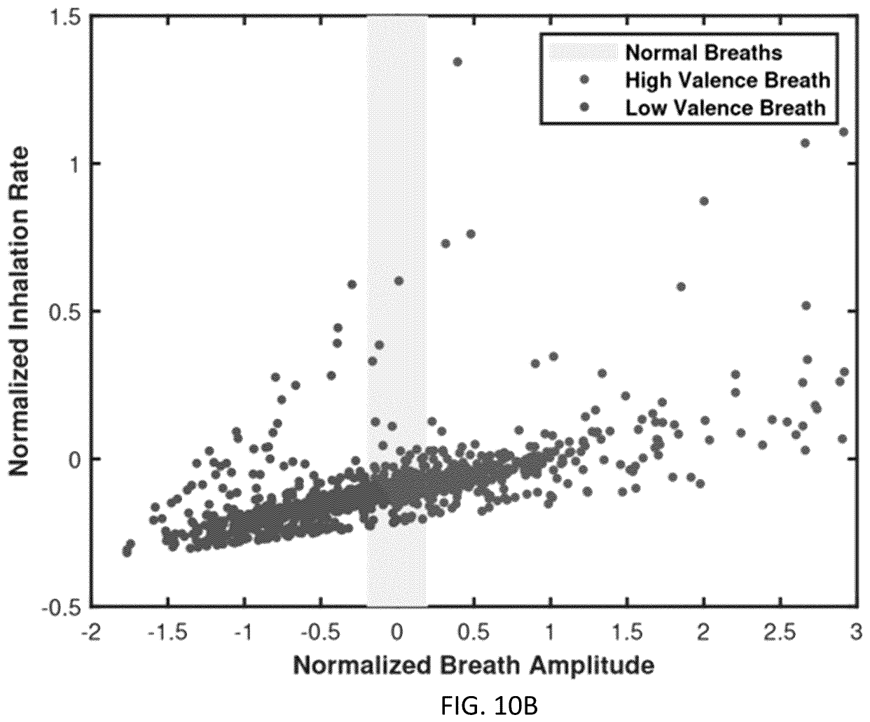

FIGS. 1A and 10A show a sample respiration waveform indicating the locations of the start of inhalation, the end of inhalation, and the end of exhalation as well as the depth of breath. The start of inhalation is the location of the initial neural stimulus corresponding to an emotional response. The inhalation rate is calculated based on the mean slope of the respiration signal between the start of inhalation and the end of inhalation. The depth of each breath is calculated based on the difference between the respiration amplitude at the end of inhalation and the start of inhalation as shown. The rate of inhalation appears to be approximately constant throughout inhalation, but the rate of exhalation appears to change prior to end of exhalation.

An example clustering for one iteration of the k-means algorithm along two features is shown in FIG. 10B. Both the breath amplitude and inhalation rate are normalized before grouping to reduce the impact of outliers on the cluster centers of the k-means algorithm. A linear correlation can also be observed between breath amplitude and inhalation rate, suggesting that larger breaths require faster inhalation times to minimize a change in the total respiration cycle time. Normal breaths are identified as having an absolute value of less than 0.25. A high density of breaths can be observed in this region which likely correspond to neutral valence periods and non-valence indicative breaths. The particular grouping shown in FIG. 10B is one which maximizes the negative correlation between the high and low valence marked point process. In other iterations of the k-means algorithm, the clustering can typically overfit to low valence breaths or fail to converge in the maximum number of allowed iterations.

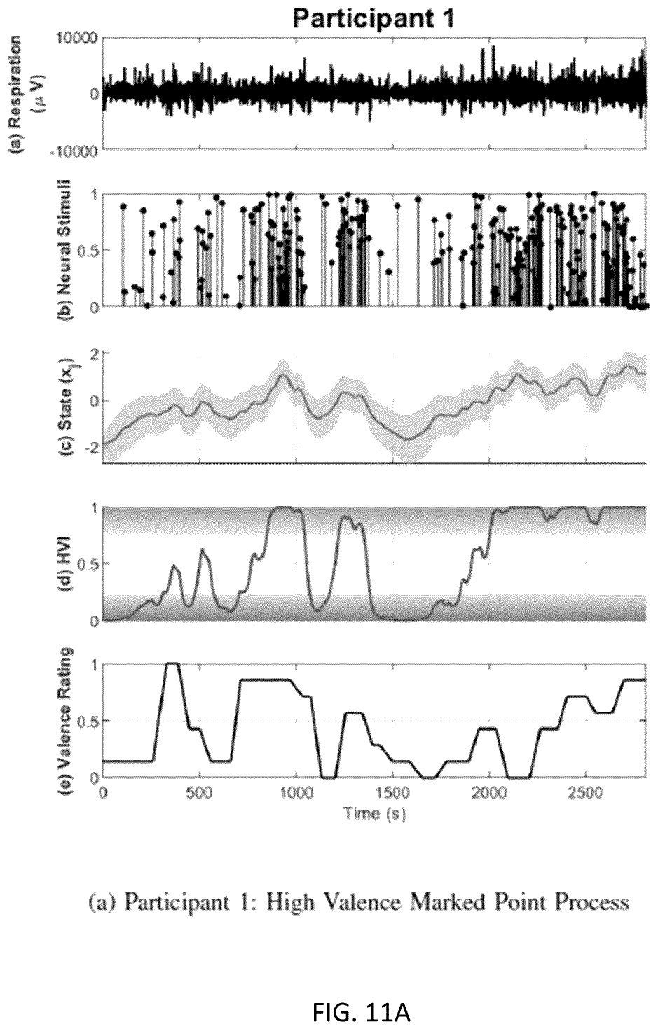

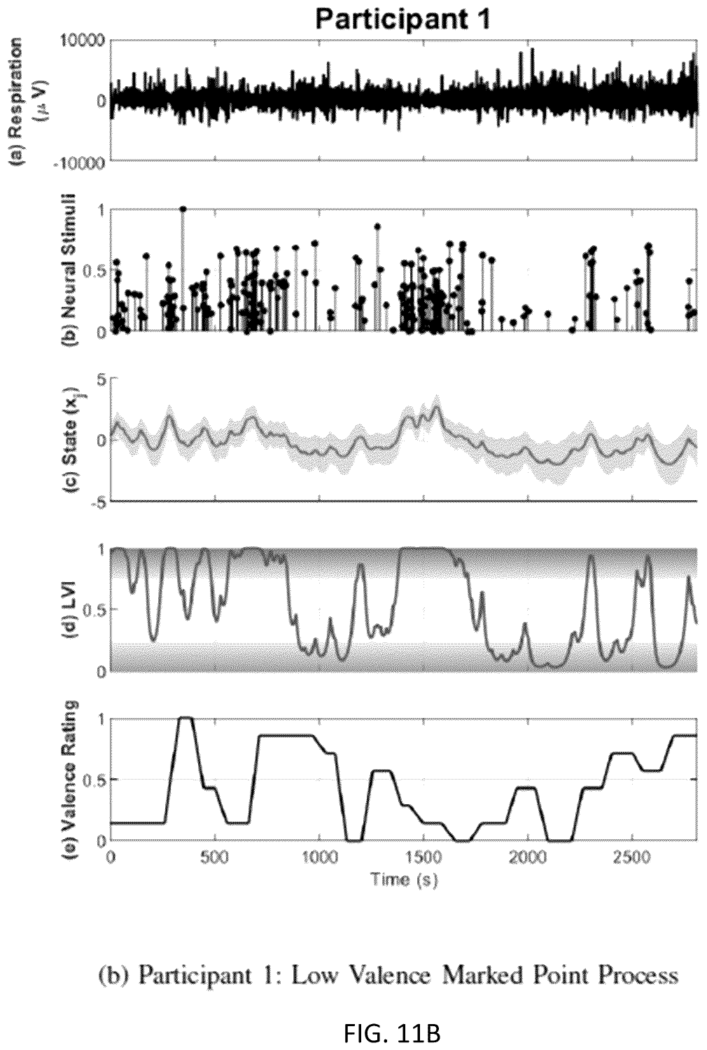

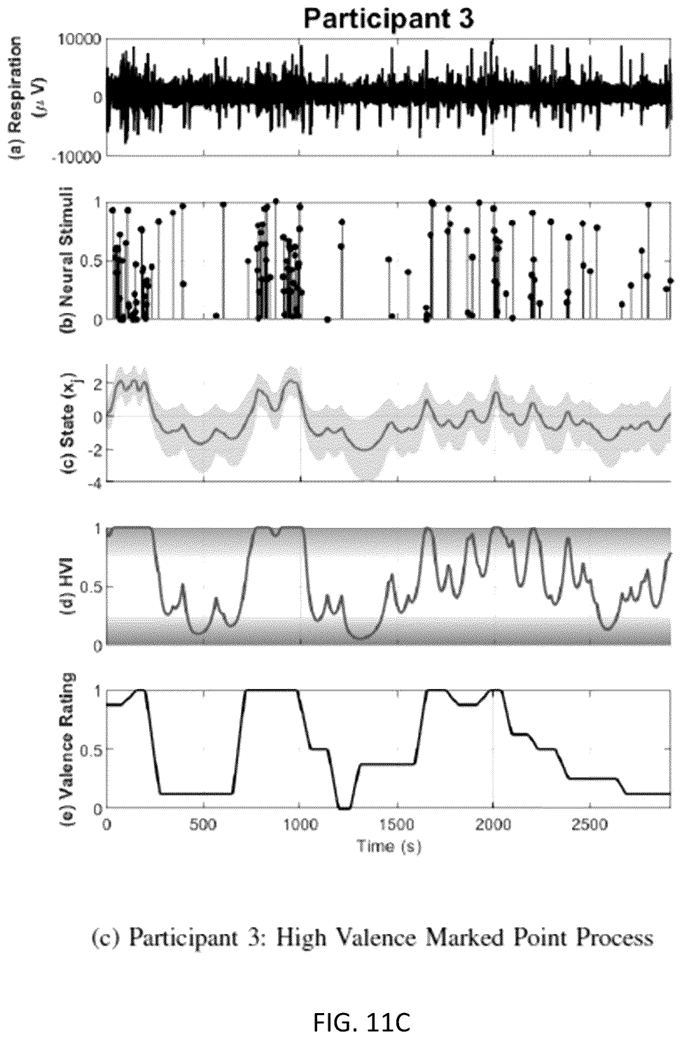

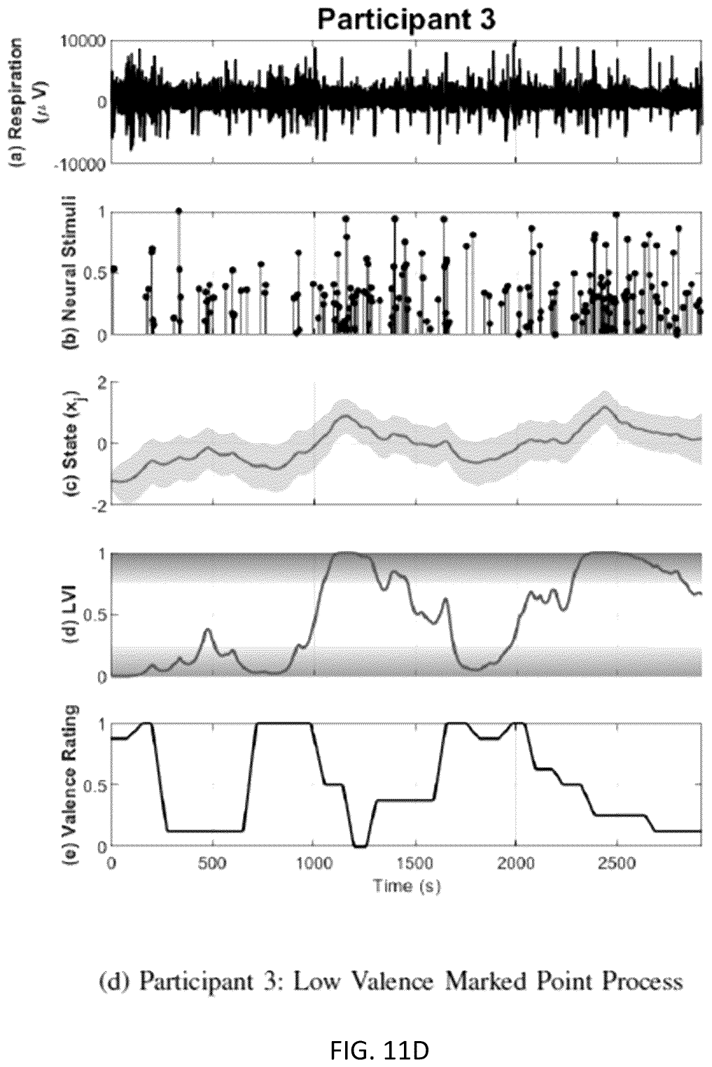

Results from marked point process generation, state estimation, and index determination are shown for both high and low valence in FIGS. 11A-D for two participants. The preprocessed and baseline corrected respiration signal is shown throughout the duration of the recordings. Large respiration amplitude spiking can be observed throughout the preprocessed signal indicating locations of potential artifacts, which are removed before generation of the marked point process. For the high valence MPP, a high density of inferred valence events can be observed during increases in valence observed in the self-reported valence ratings. The sparsity and magnitude distribution of valence events varies significantly between participants. MPP results for participant 1 show a higher occurrence of valence events compared to participant 3. In comparison, MPP results from participant 3 contain fewer stimuli, but have higher mean magnitudes than participant 1. For the low valence MPP, a high density of valence events was observed associated with the decreases in valence shown in the self-reported ratings. Compared to the high valence indicative events, the low valence marked point process is less sparse and contains a larger amount of low amplitude impulses. Valence events are also observed in between trials, in which neutral clips evoking no emotional response are shown.

The high and low valence states and associated indices are also shown and compared to the self-reported ratings. The valence state and index increase as the magnitude and occurrence of valence events increase. Periods of very high and very low valence are indicated in the plot of the valence index. The high valence index captures the peaks associated with high valence period in the self-reported ratings but fails to capture several decreases in valence. These decreases are partially captured in the low valence index. For example, in participant 1, small decreases in valence can be observed at the end of the recording. The low valence index contains peaks corresponding to the same drops in valence that are more pronounced than the decreases observed in the high valence index. For participant 3, the high valence index appears to be faster varying, while still capturing the high valence periods found in the reported valence ratings. The low valence index is smoother and appears to correspond to the periods of low valence.

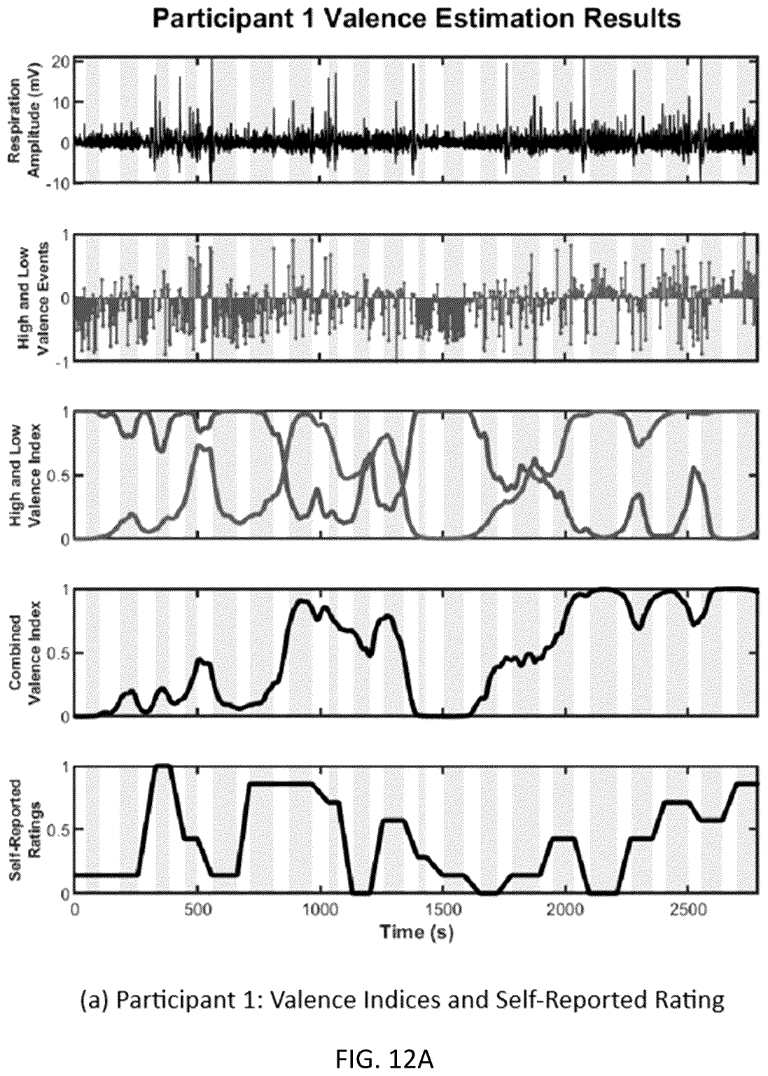

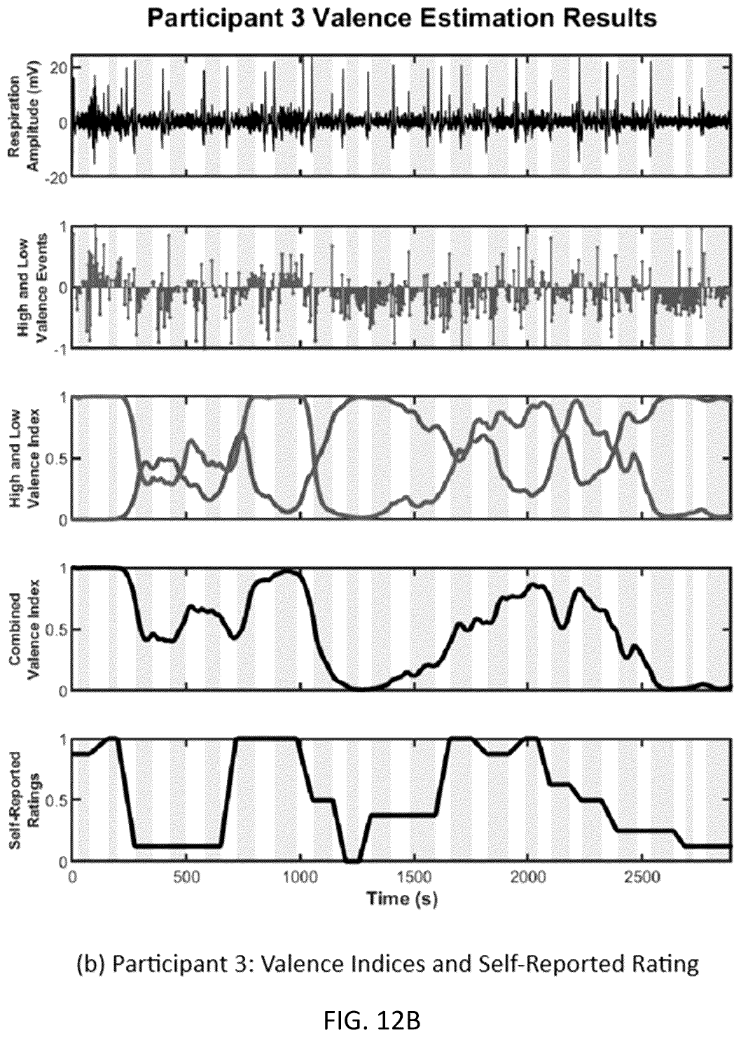

FIGS. 12A-12B show the combination of the high and low valence indices into a single index and comparison to the interpolated self-reported ratings of the participants. The high valence index captures the trends in the self-reported valence ratings in terms of high and low valence periods independently. The low valence index better captures decreases in valence and refines the predicted valence once it is combined with the high valence index. Both indices capture similar trends in valence in terms of indication of a high or low valence period. The combined valence index provides an improved estimate of valence considering the information unique to either index. The self-reported valence ratings are also shown for each participant along with the trial periods in which video clips are shown. Though the ratings are interpolated between trials to generate a continuous value for reported valence, these periods should correspond to decreases in valence corresponding to the showing of a neutral valence clip. However, the combined valence index fails to capture rapid changes in valence and, instead, follows a general trend in valence dependent on the video clips shown.

For participant 1, the low valence index indicates two lower valence periods corresponding to the 17th and 19th trial that are not as apparent in the high valence index as significant decreases in valence. The combined index considers both indices and, instead, shows a decrease to neutral valence corresponding to the peaks in the low valence index. This slight decrease in valence is also reported by the participant in both trials. Likewise, for participant 3, extraneous peaks detected in the high valence index were observed being corrected by low valence periods detected in the low valence index. However, in either case, the low valence index does not appear to be a better indicator of changes in valence than the high valence index. Instead, it appears to refine and correct the estimation of the high valence index. In some cases, as in trial 16 of participant 1, both valence indices misidentify a low valence period as a high valence period. This could be caused by manual correction of respiration by the participant, error in valence rating, or a conflicting response to the stimulus (i.e. feeling amused by a horror movie). In many participants, an increase was observed in estimated valence at the end of the recording, which could be a result of confounding factors in experimental setup.

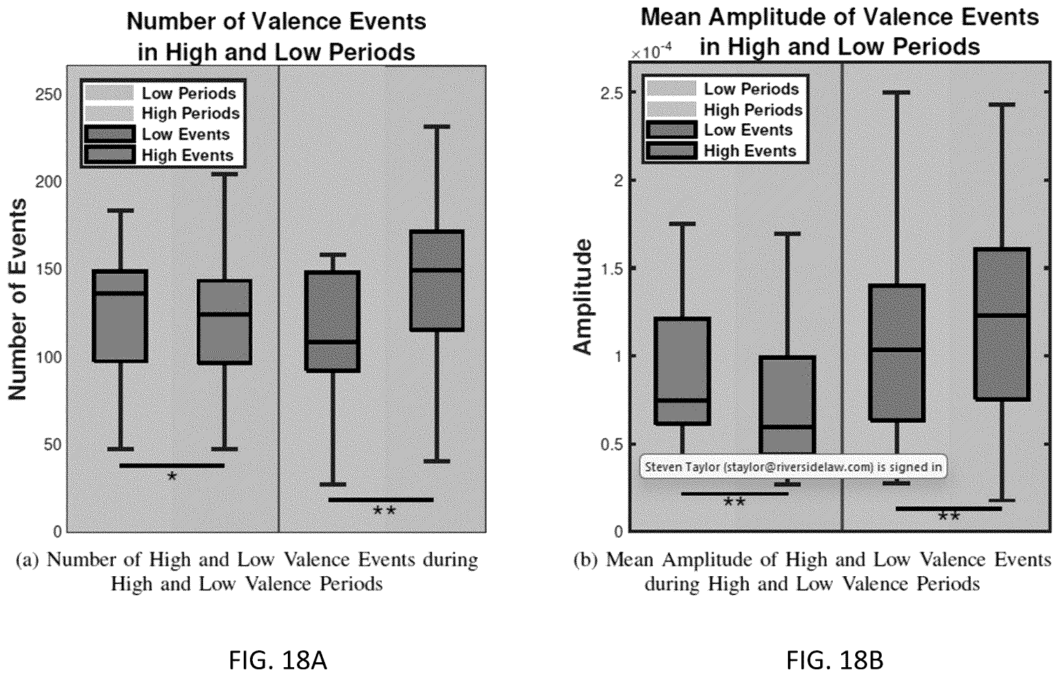

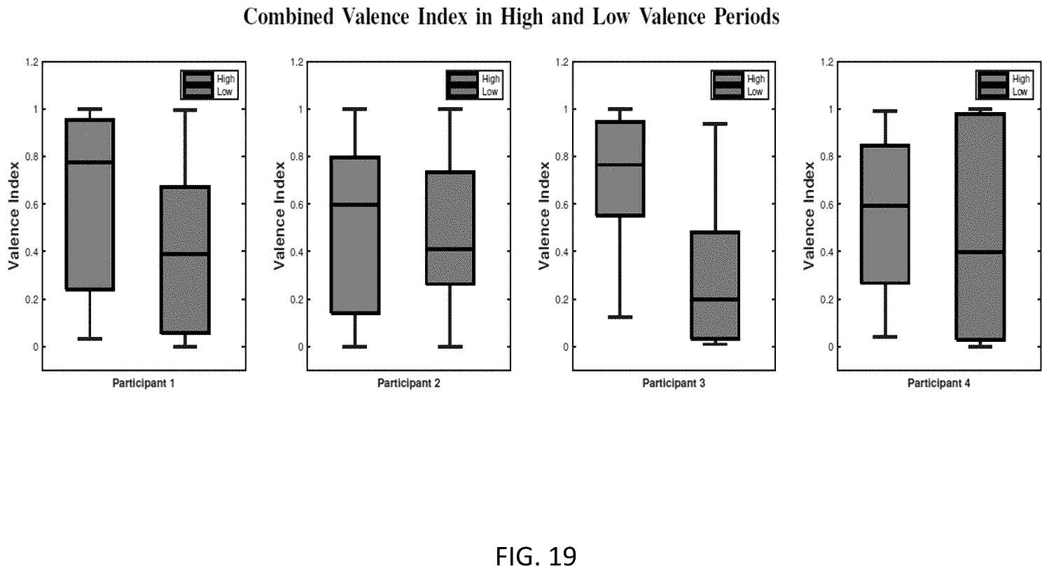

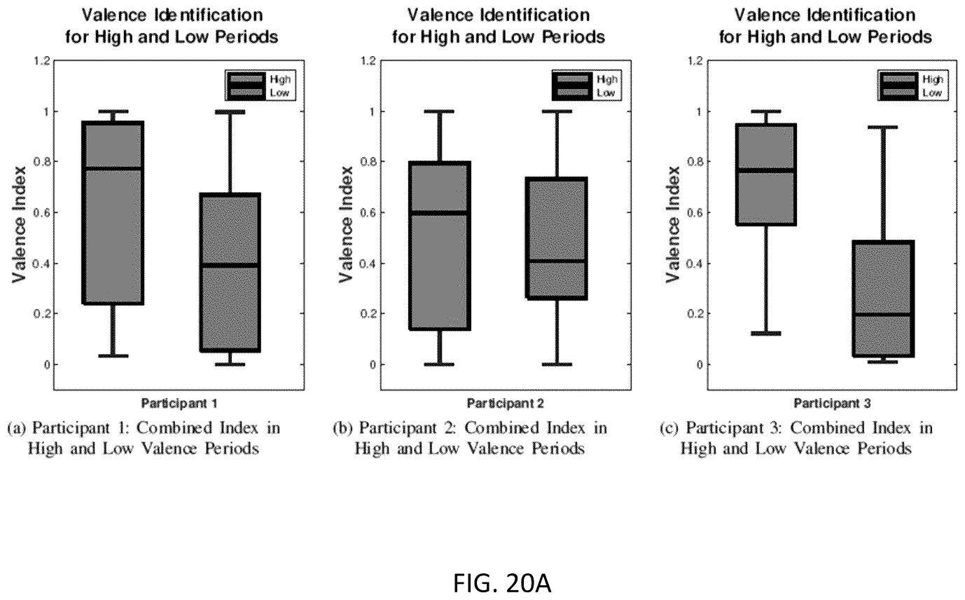

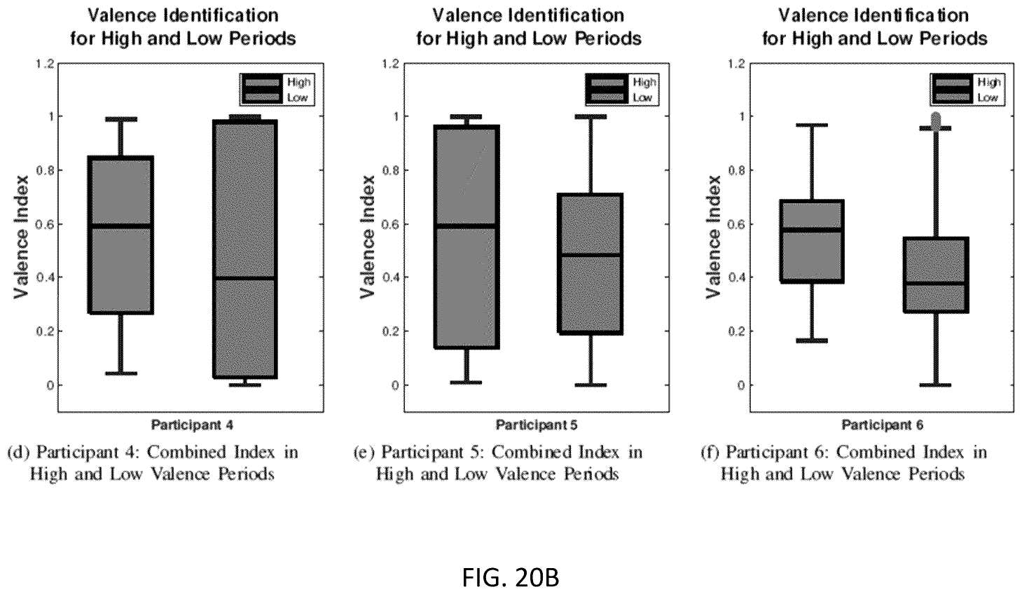

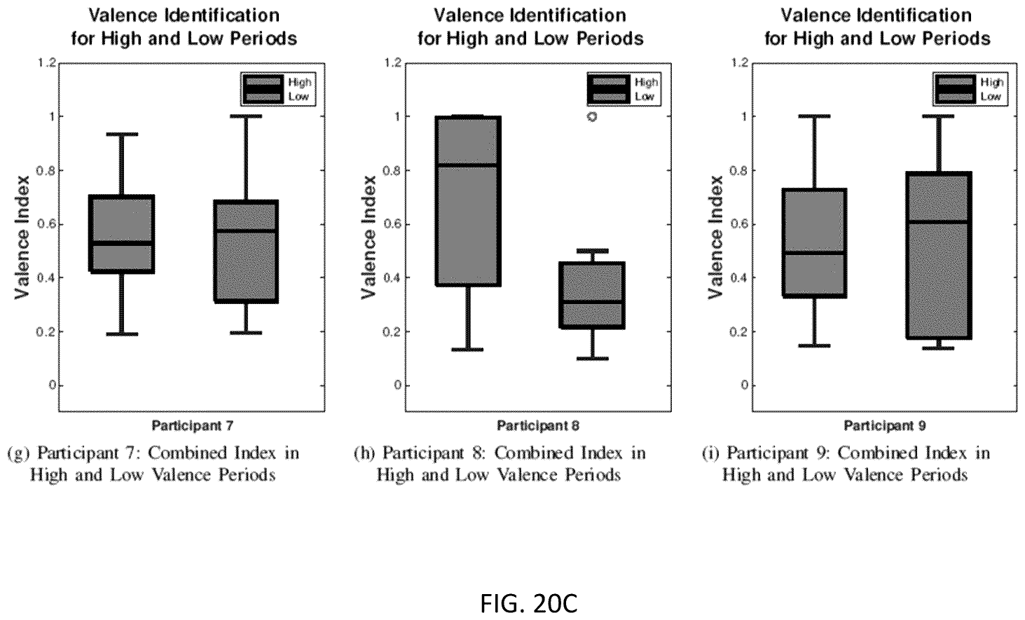

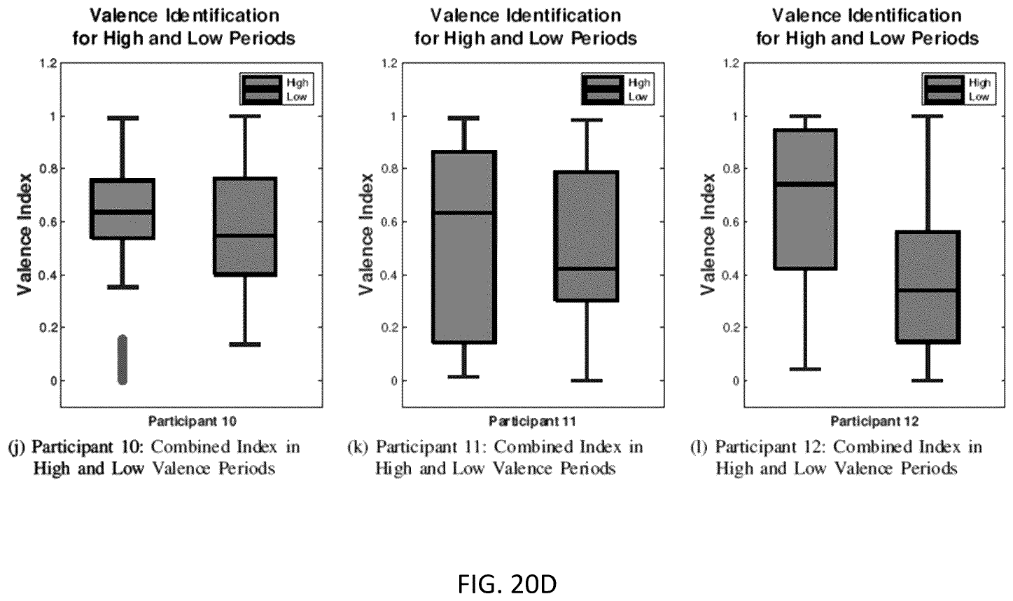

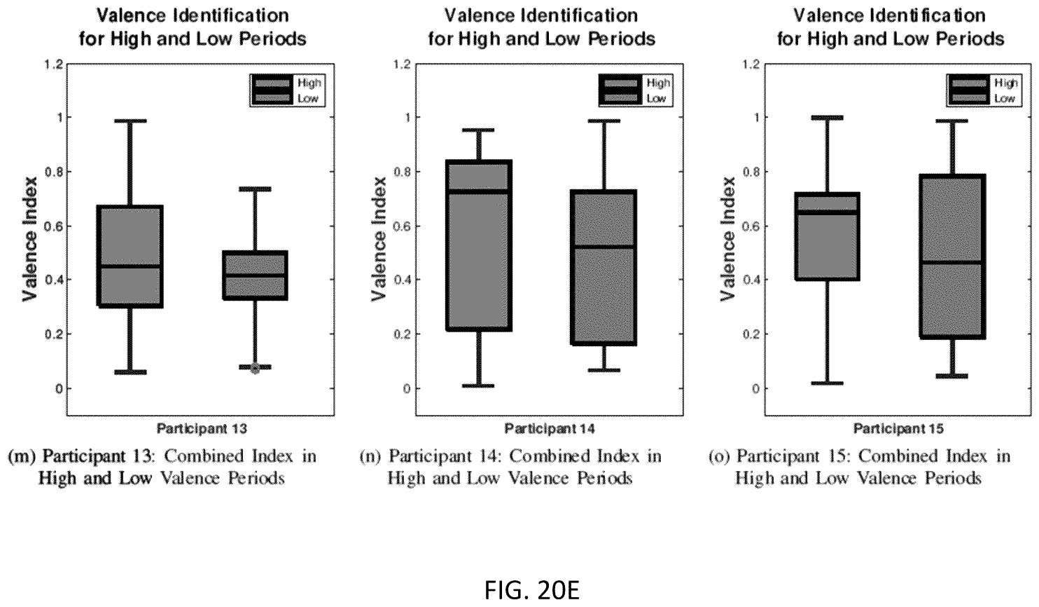

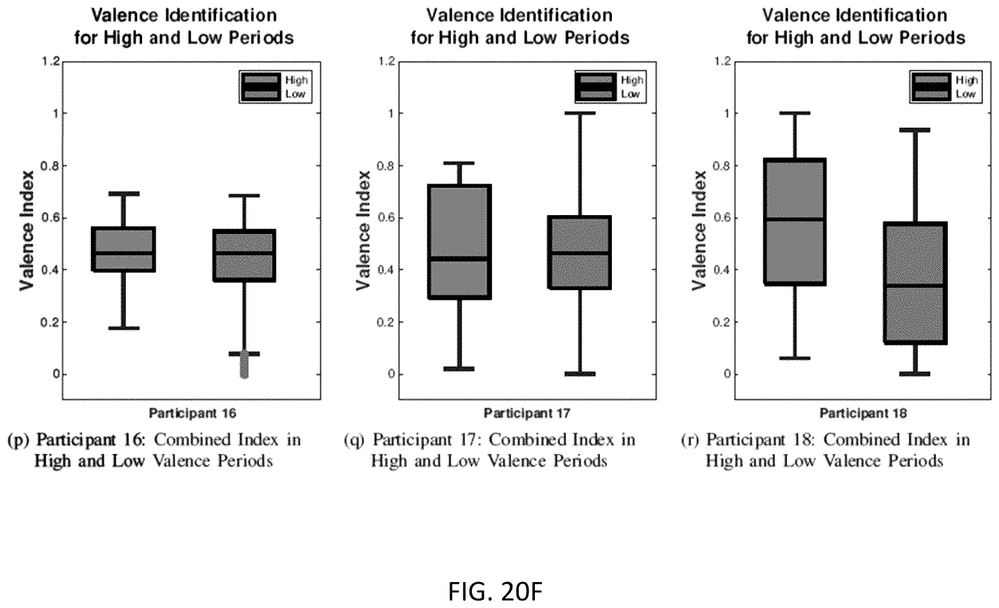

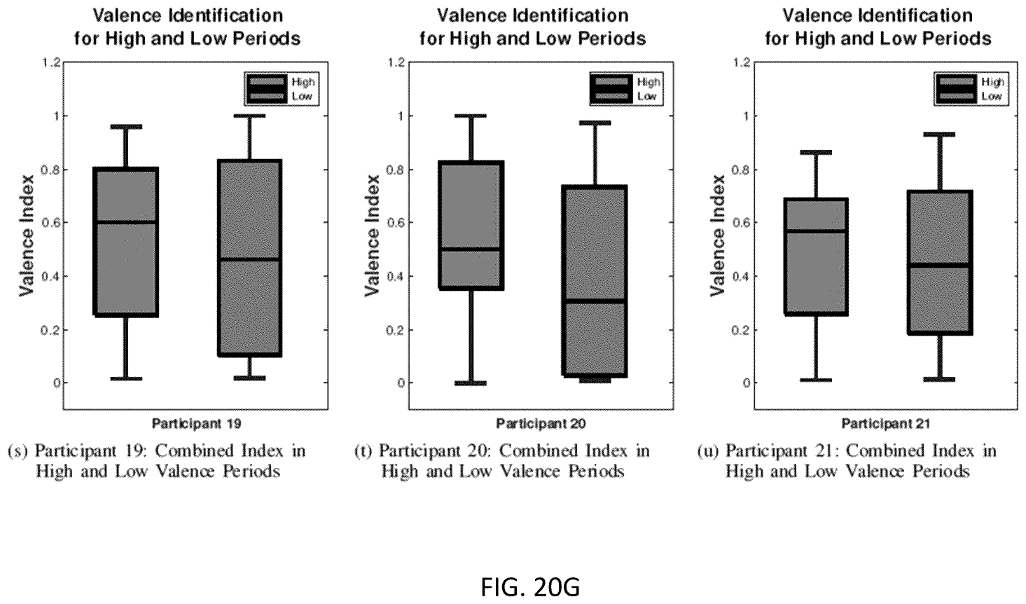

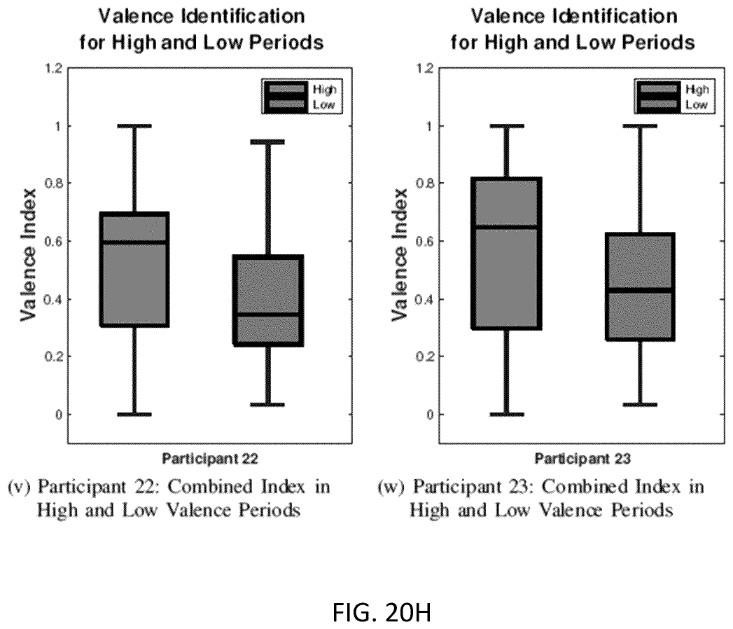

FIGS. 18A-18B and 19 show box plots of valence event parameters and valence estimation results for high and low valence periods. Based on the event parameters, the low valence event detection performs better in terms event detection, whereas the high valence event detection benefits more from the consideration of the amplitude of events. Regardless of the statistical significance of the differences in high and low valence event detection, the estimation performs well if the number and magnitude of events is larger for the high and low MPPs in their respective periods. The overall performance of the valence estimation is measured using the ability to identify high and low valence periods based on the combined valence index. For all but 6 participants (2, 7, 9, 17, 19, and 21), the valence index was able to distinguish high and low periods with a p-value of less than 0.05. For several of these participants, the results were significantly improved by flipping the high and low valence MPPs, meaning the assumption that high valence corresponds to higher amplitude breaths has failed for these participants. For two participants (7 and 11), contained missing video clips and shorter respiration signals. By changing the threshold of considering high and low valence periods from 0.5, the quality of the algorithm can be better assessed for specific applications.

For the purposes of statistical analysis, potential periods of high or low valence based on self-reported valence ratings were identified. A threshold of 0.5 was set for the normalized and rescaled valence ratings to distinguish high and low valence periods. For both periods, the corresponding valence index is measured in each time bin throughout the entire recording. The duration of the recording following the final video clip being shown is removed from analysis as the participant's valence levels are unknown, but this has frequently been observed to contain a large amount of high valence events for many subjects. This could possibly be a result of issues with experimental setup, confounding factors, or motion artifacting. The separation between high and low valence periods can be impacted by the threshold selected. For example, choosing narrower thresholds for identification drastically reduces the number of total valence events in either period, resulting in the magnitude of the event being more informative than the incidence of events. In the case of video clip viewing, the degree of valence changes may be less significant than other applications, such as mental health monitoring.

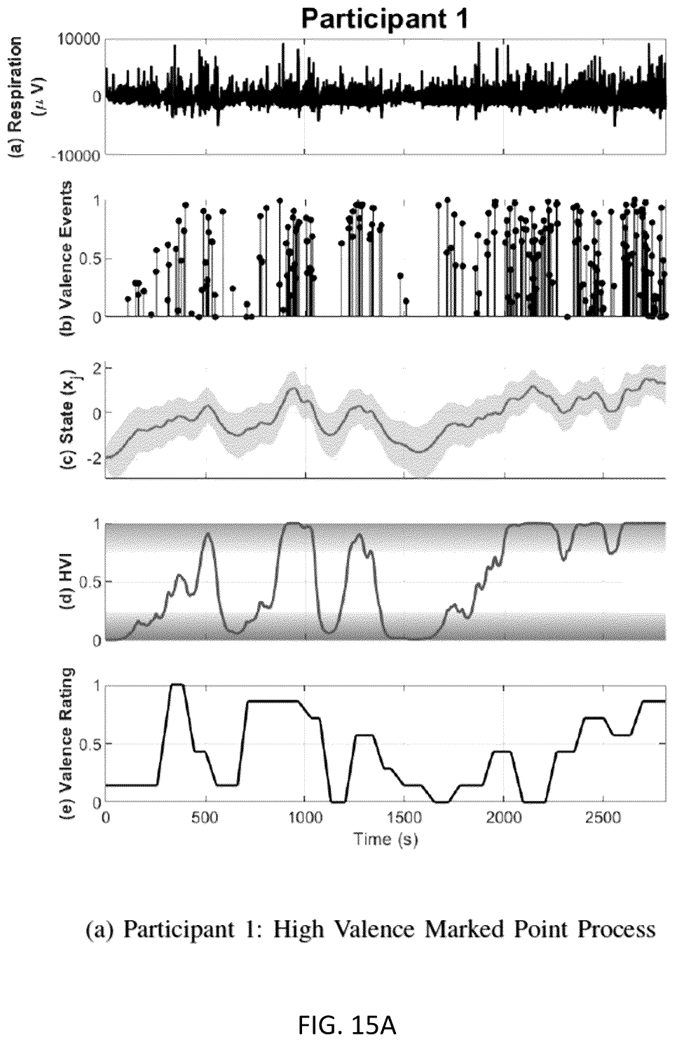

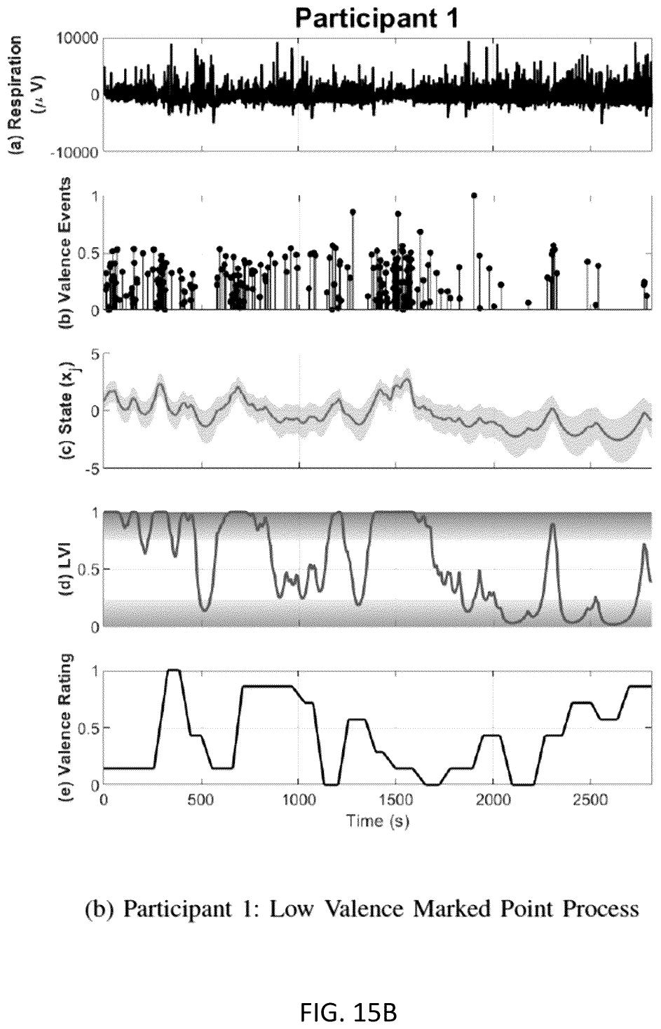

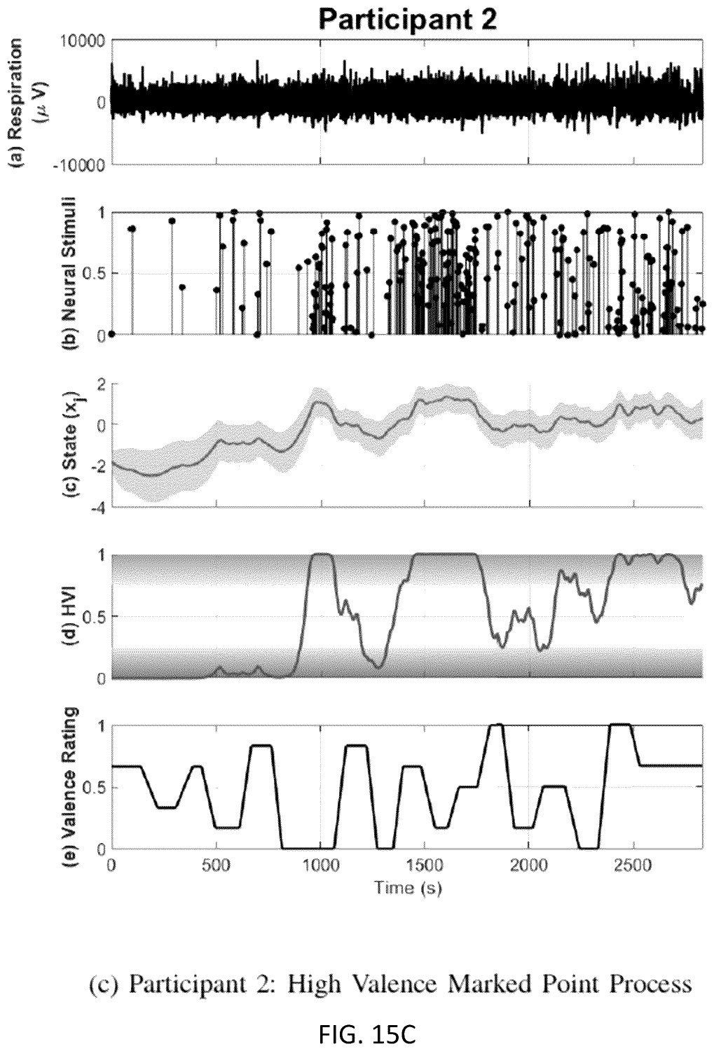

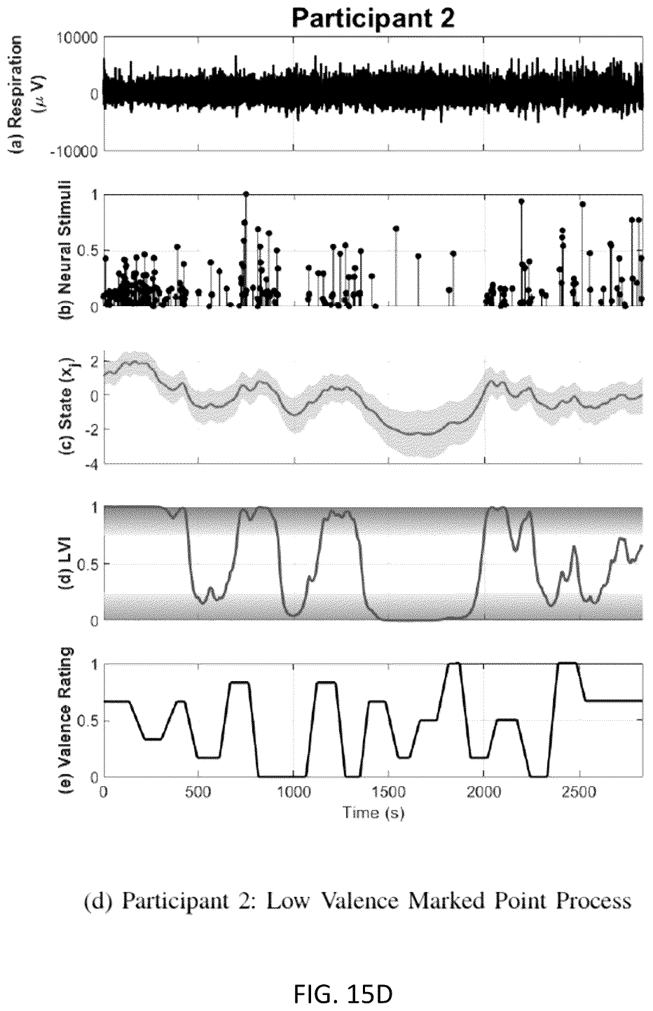

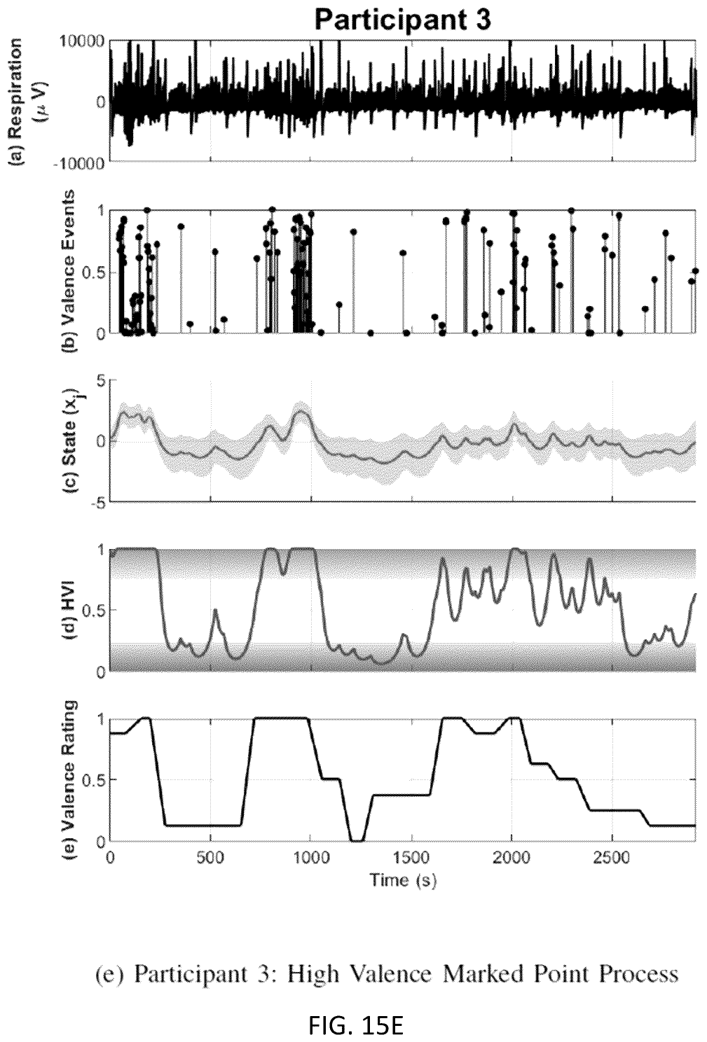

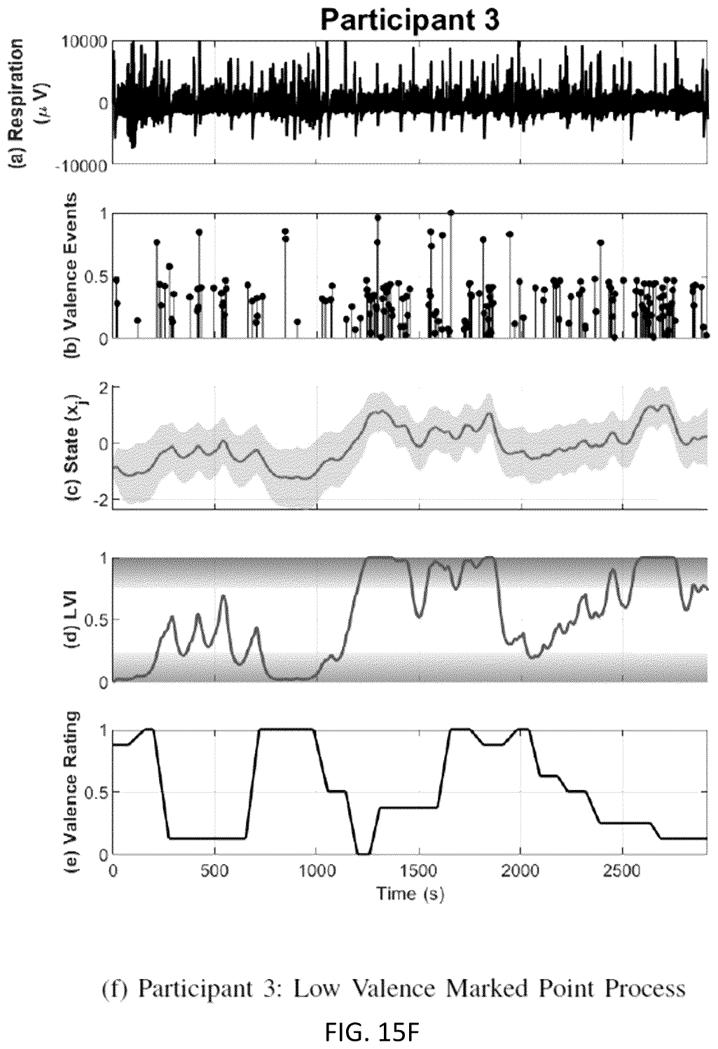

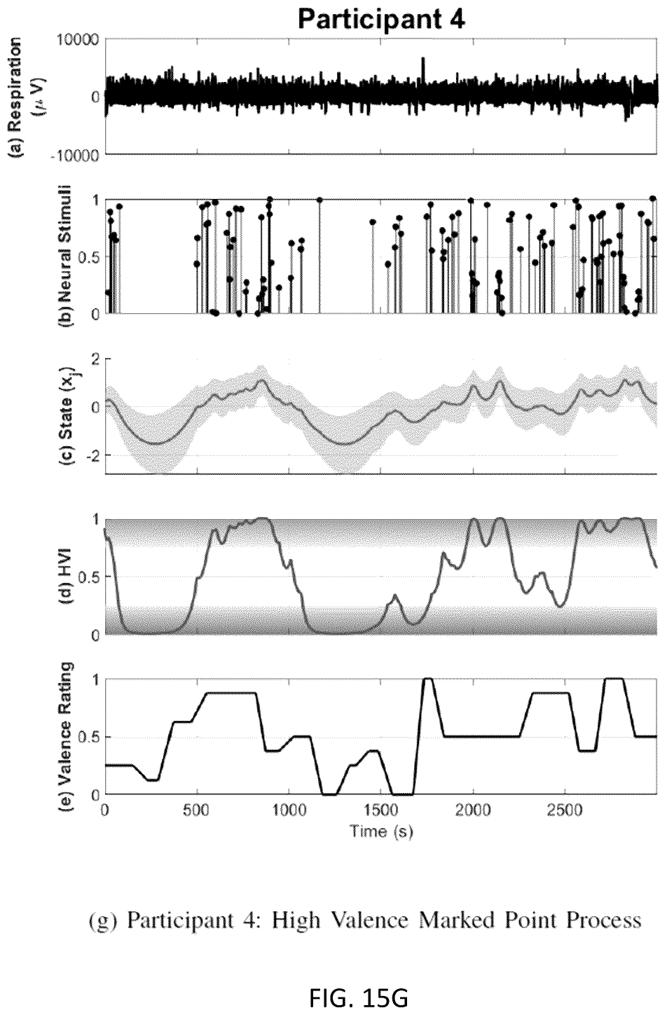

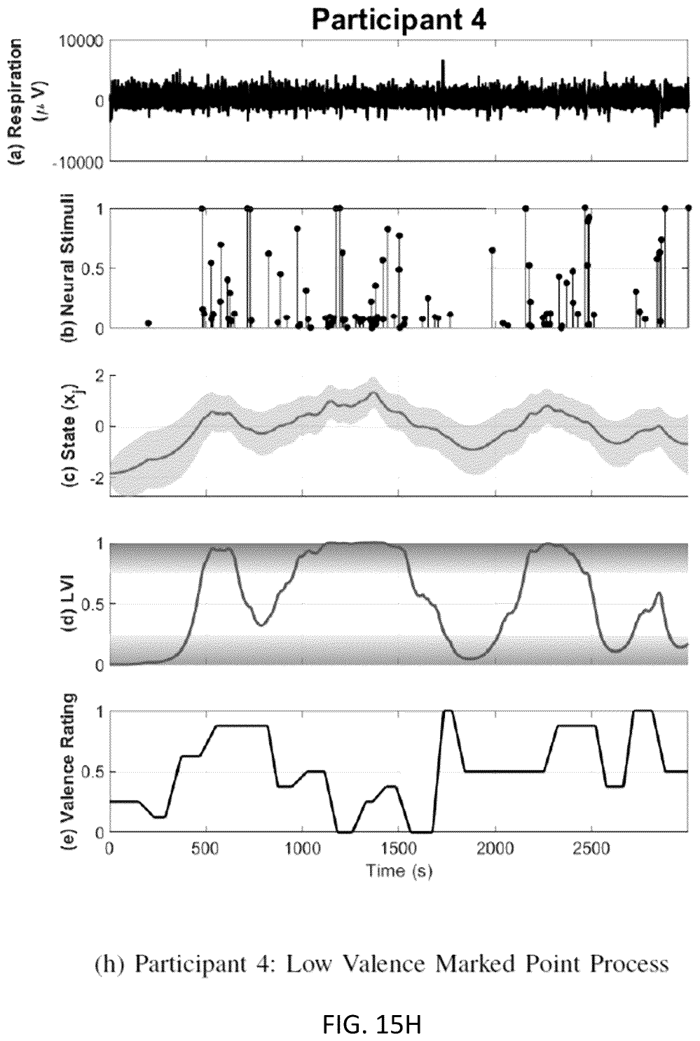

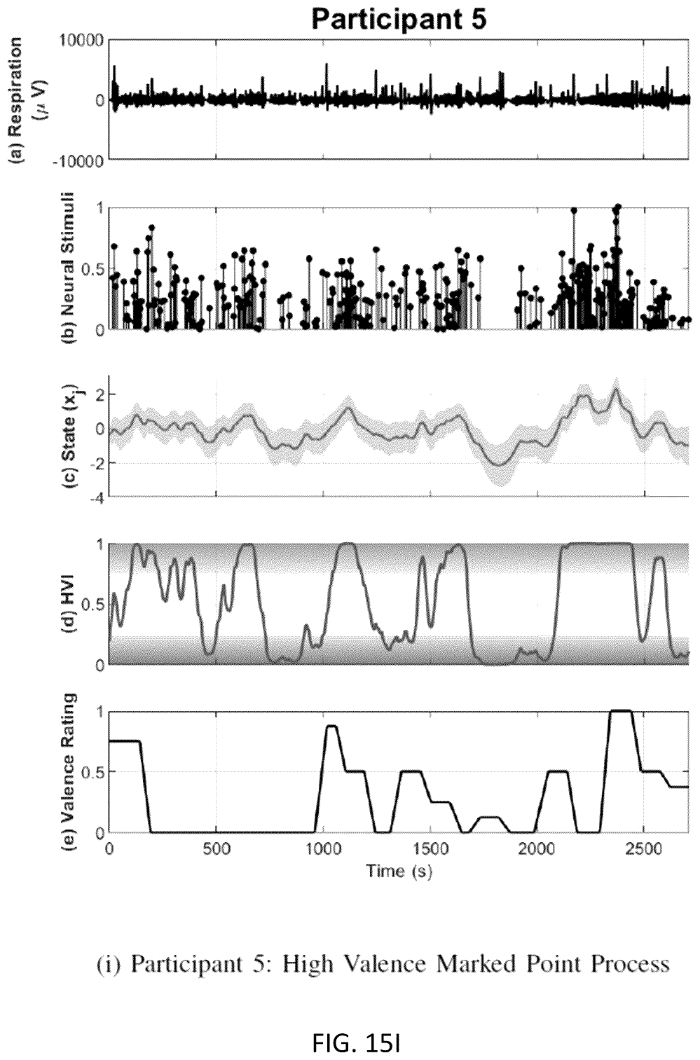

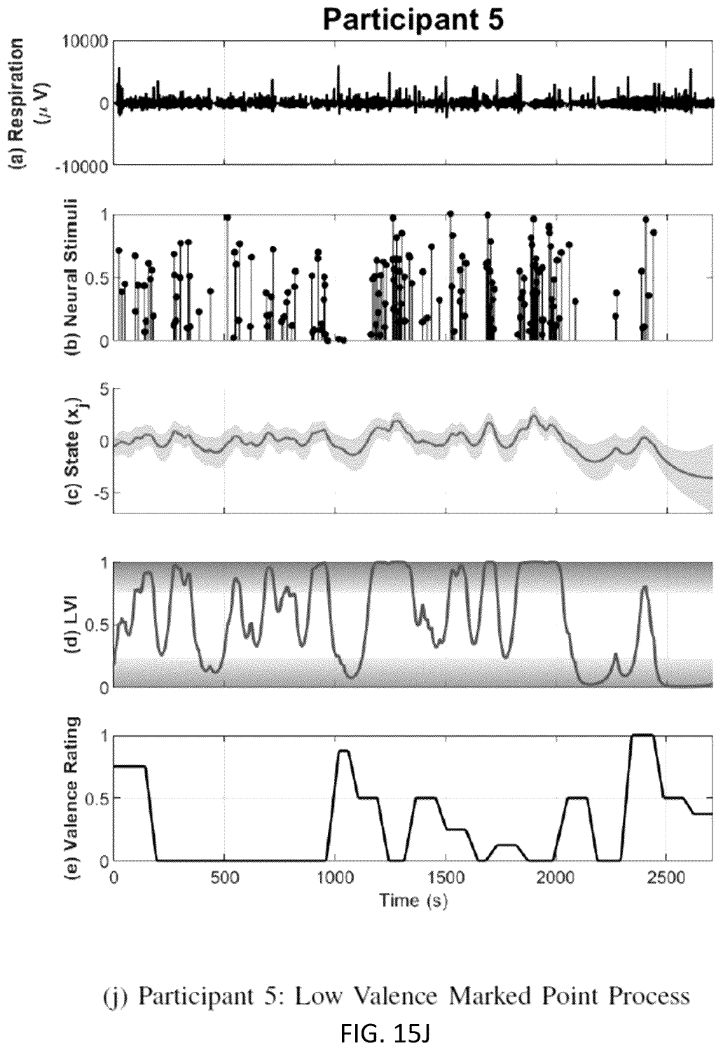

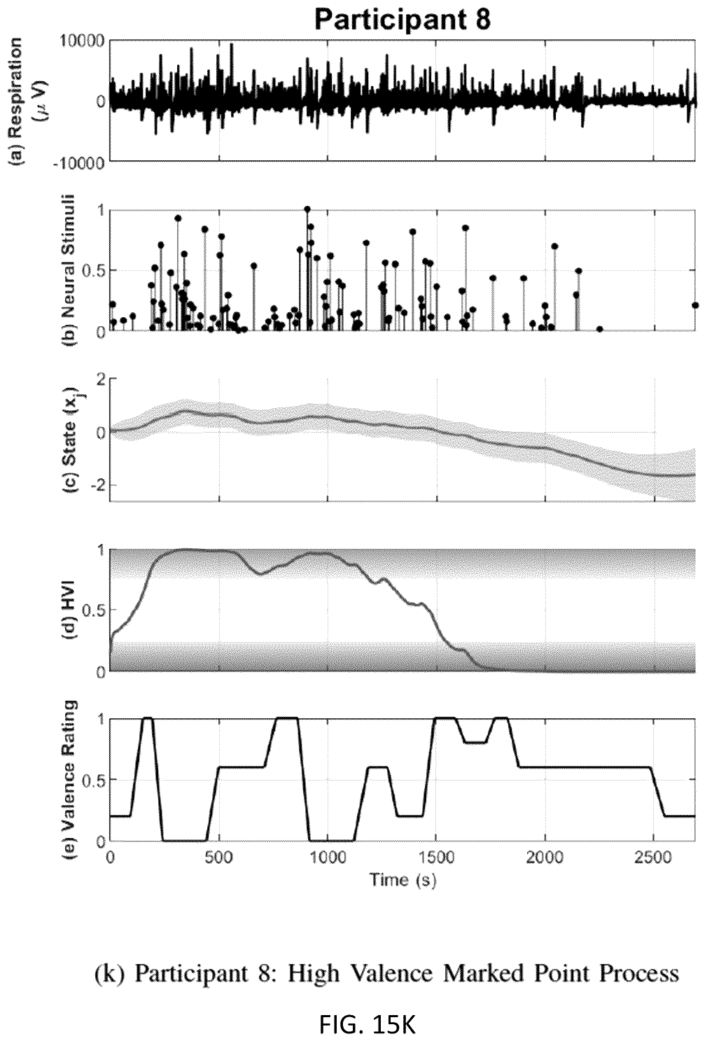

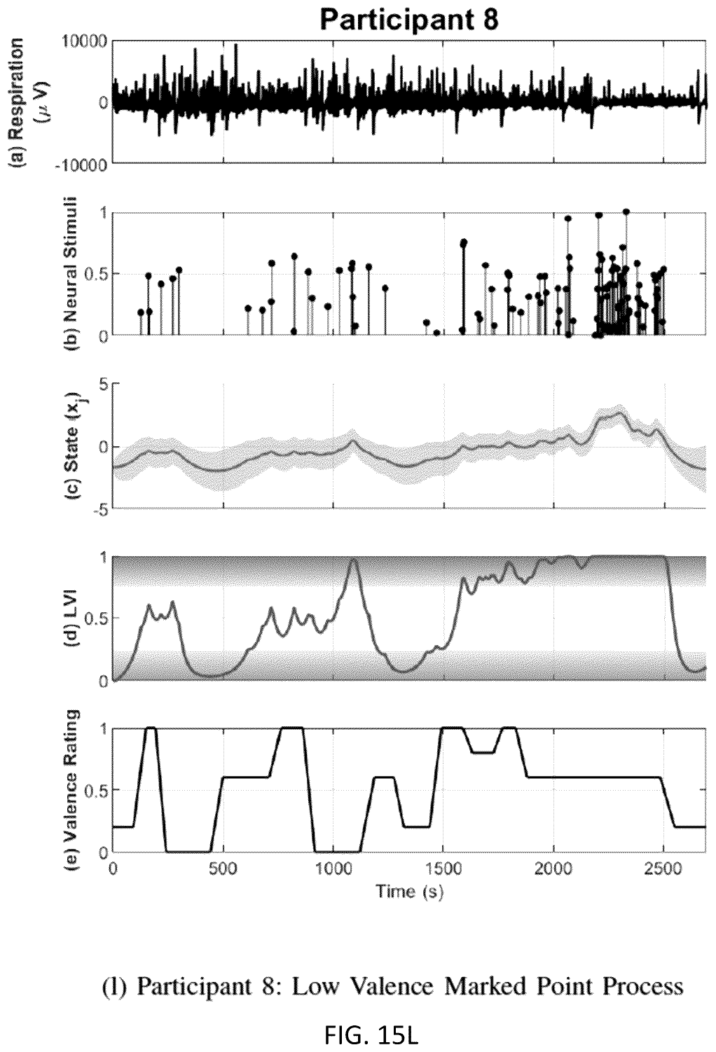

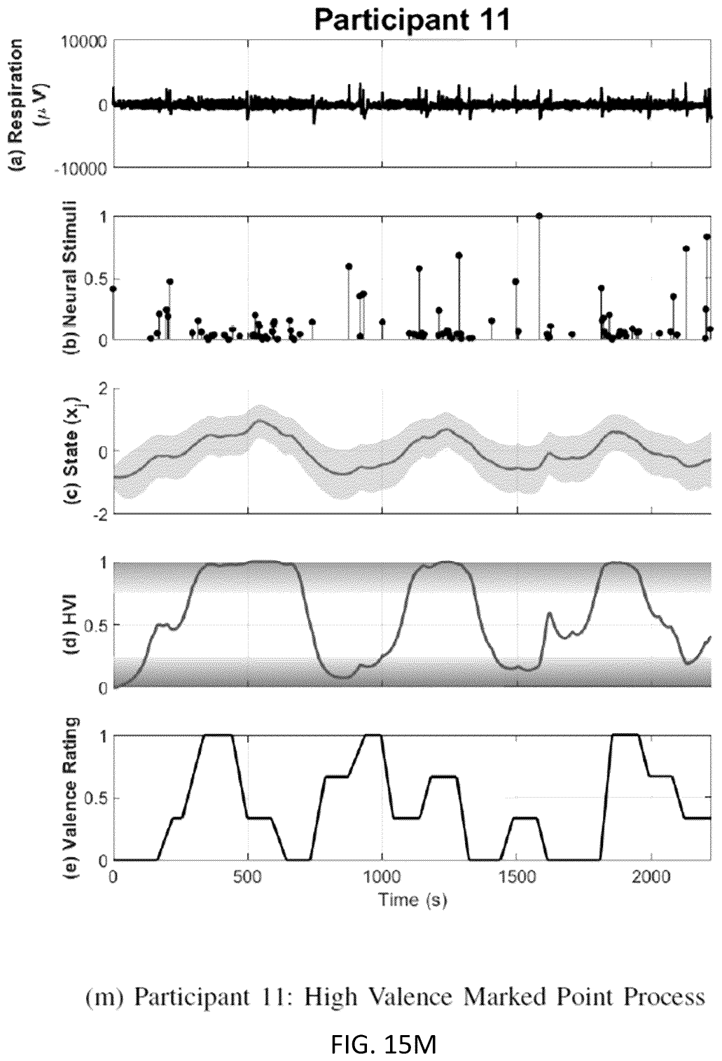

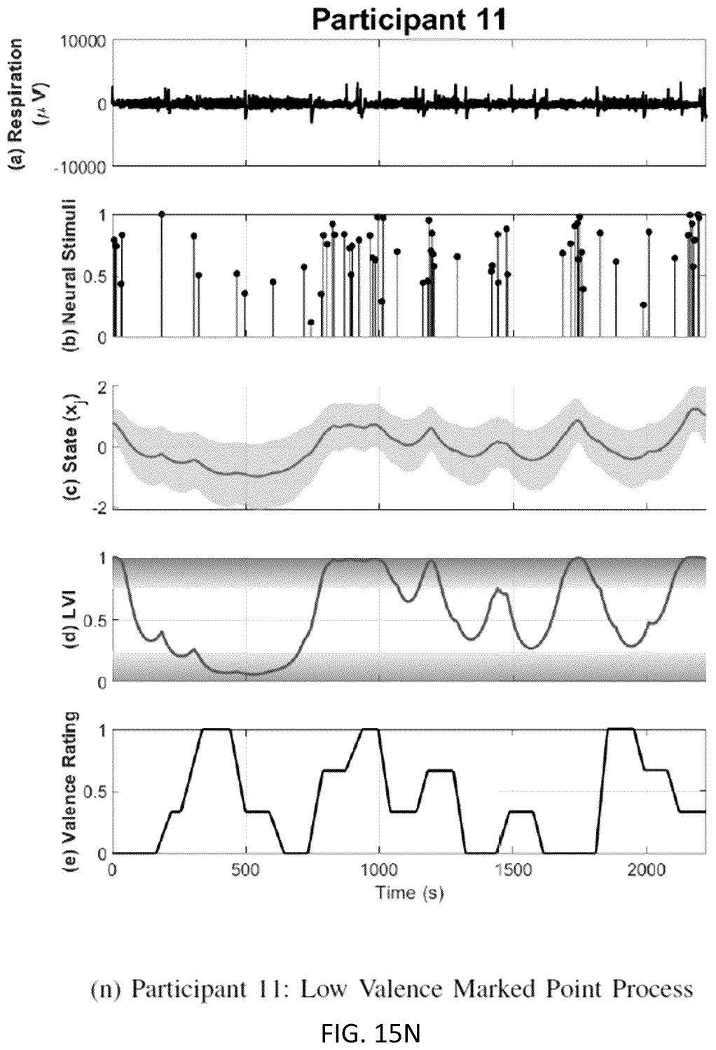

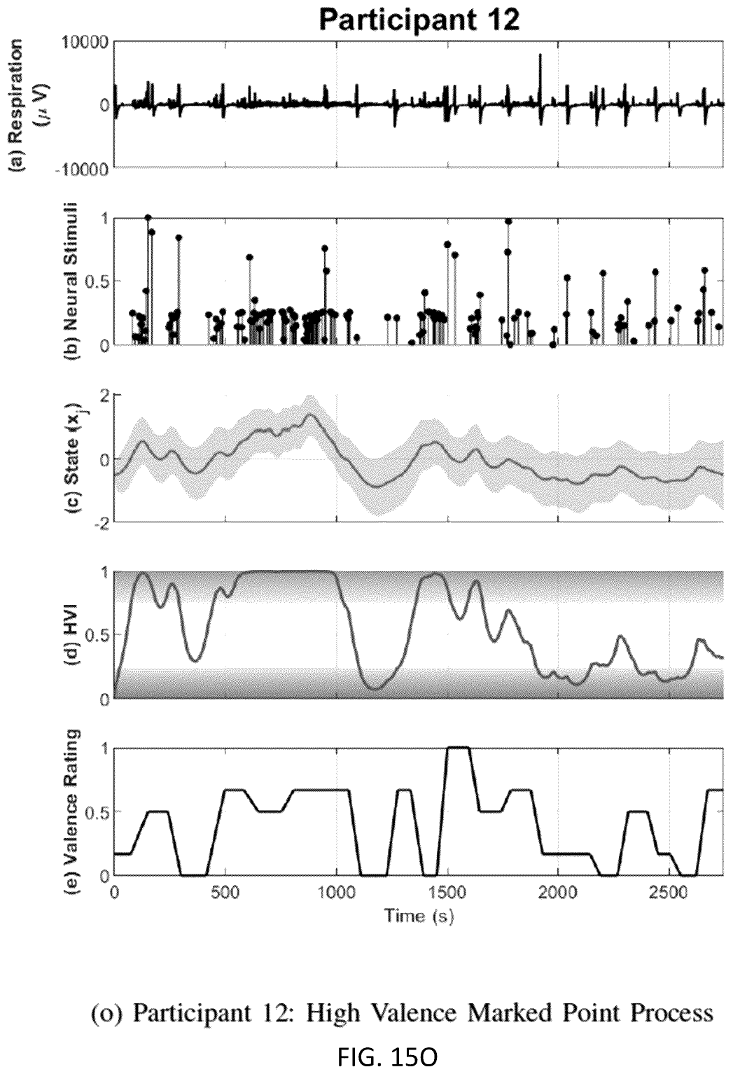

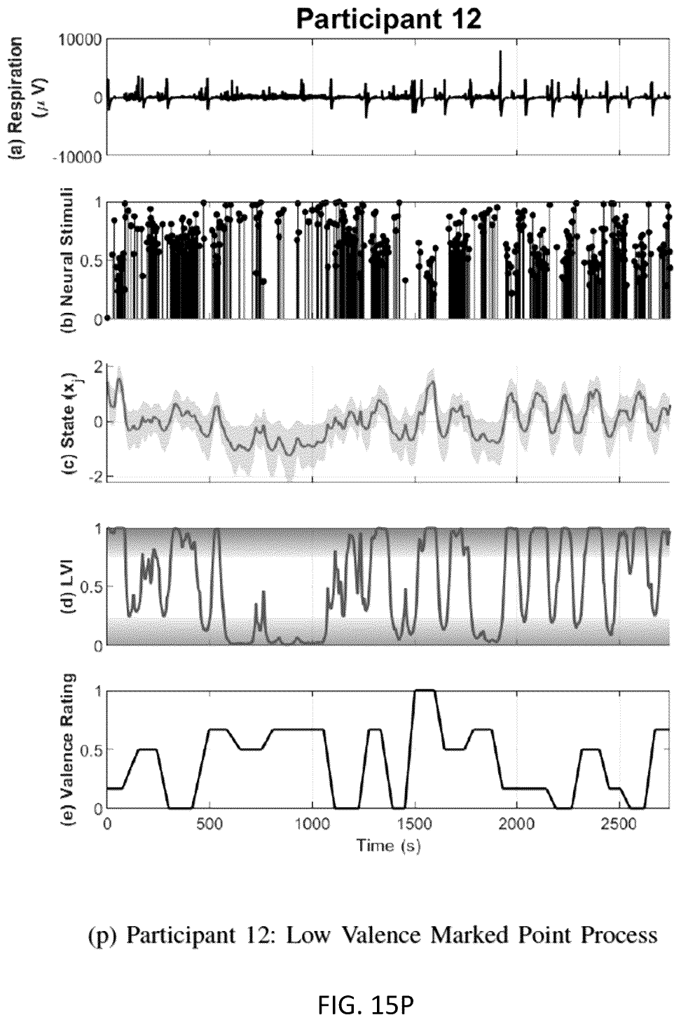

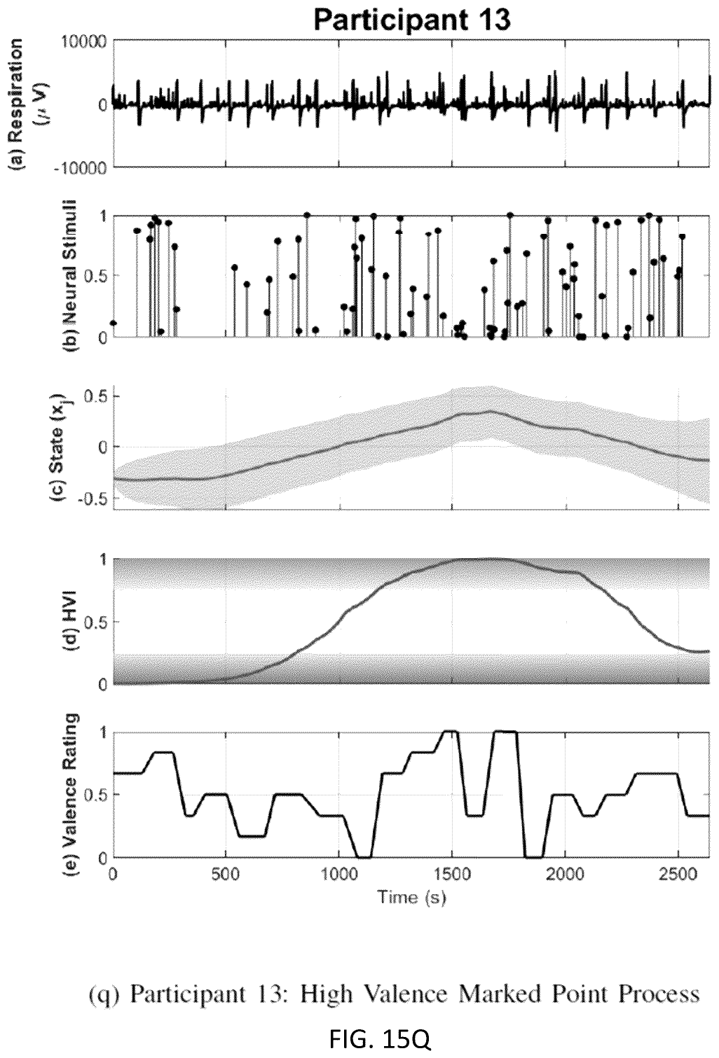

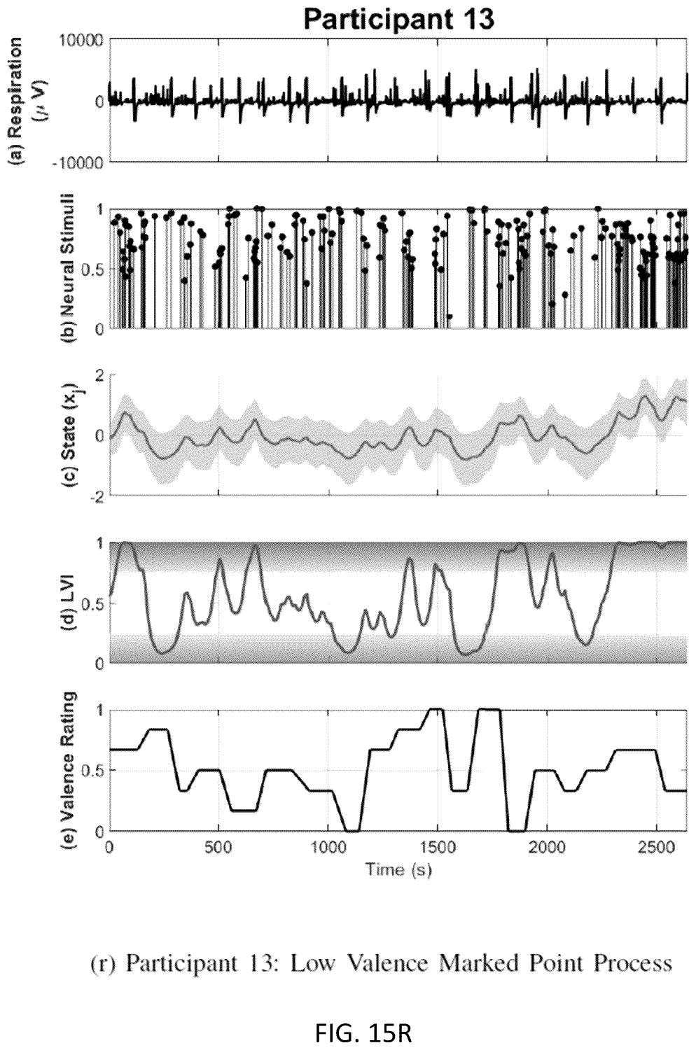

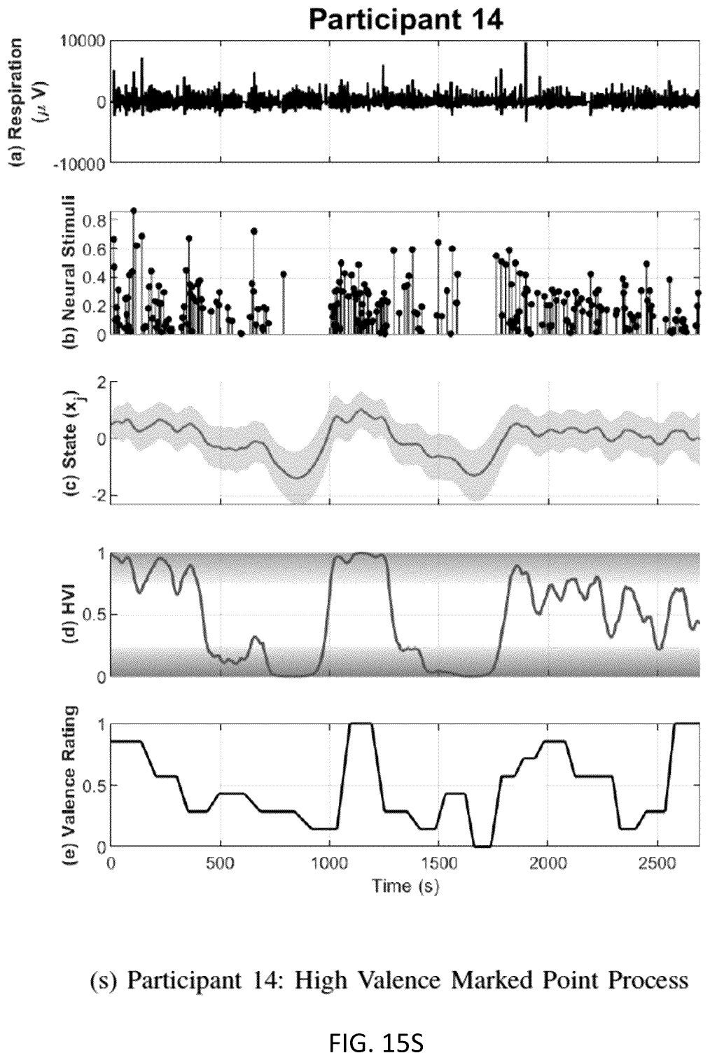

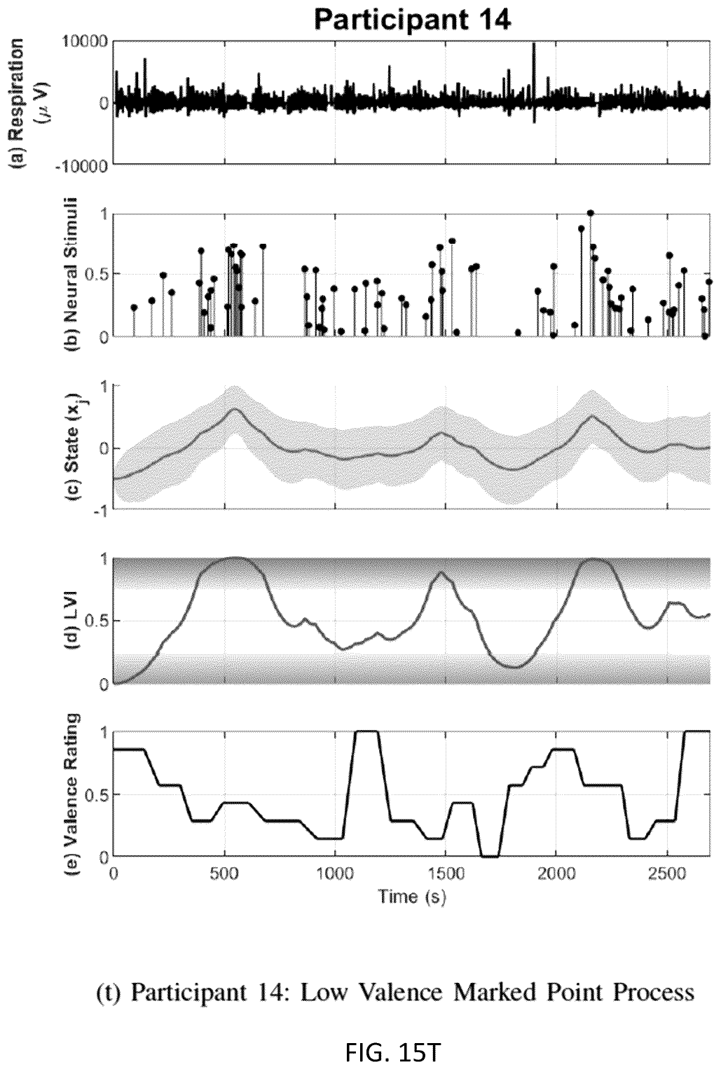

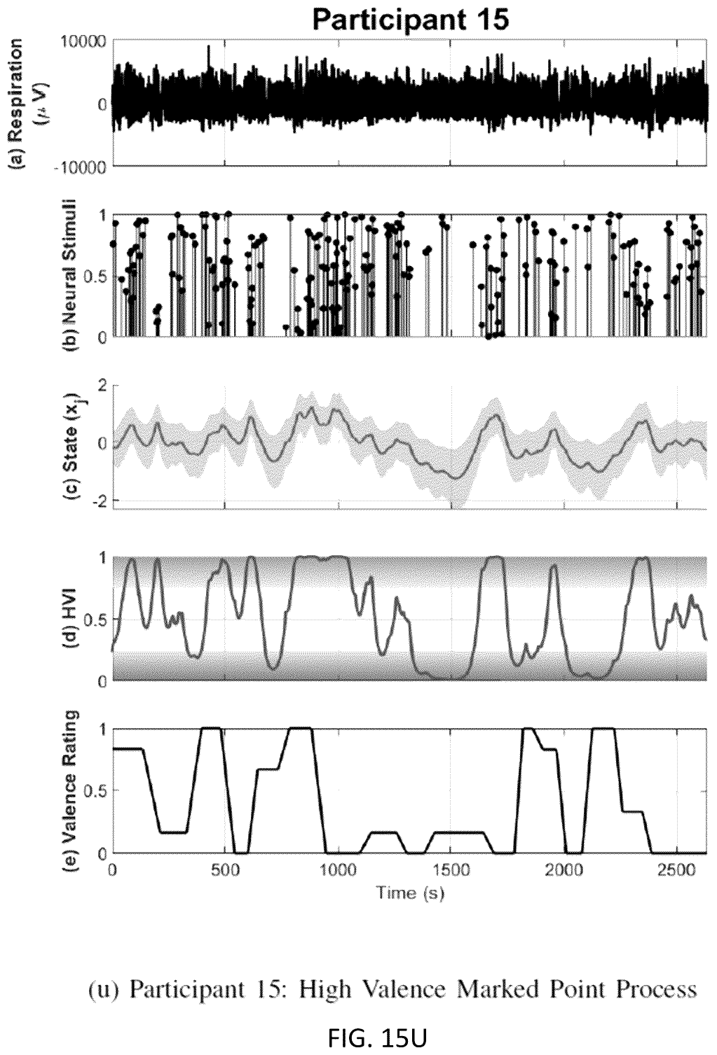

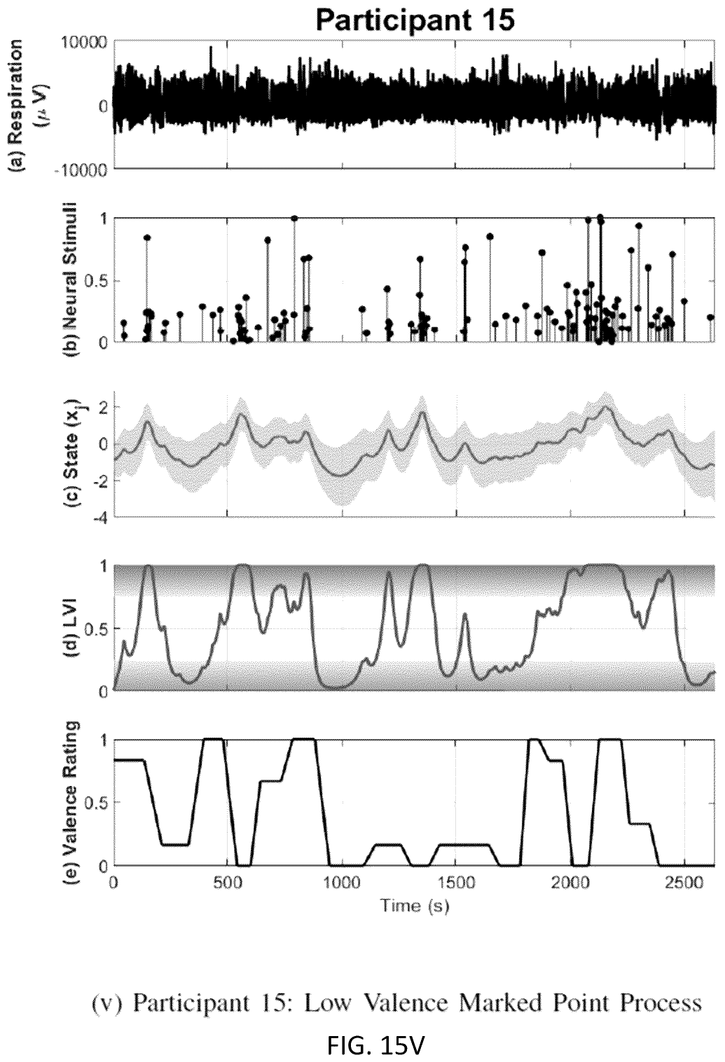

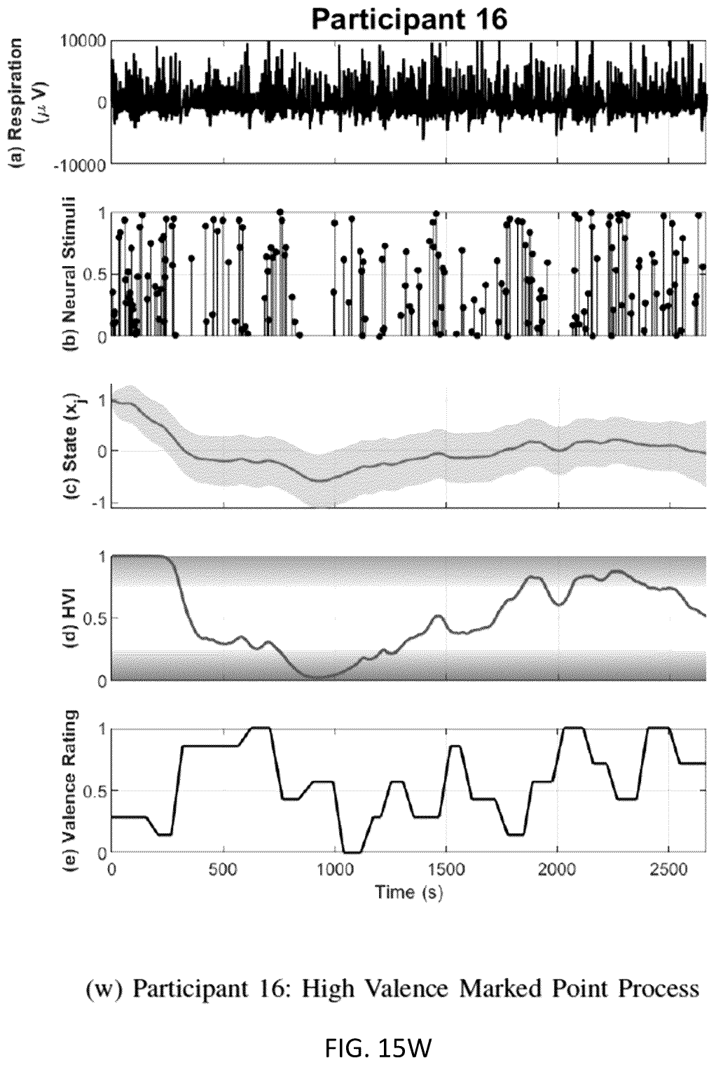

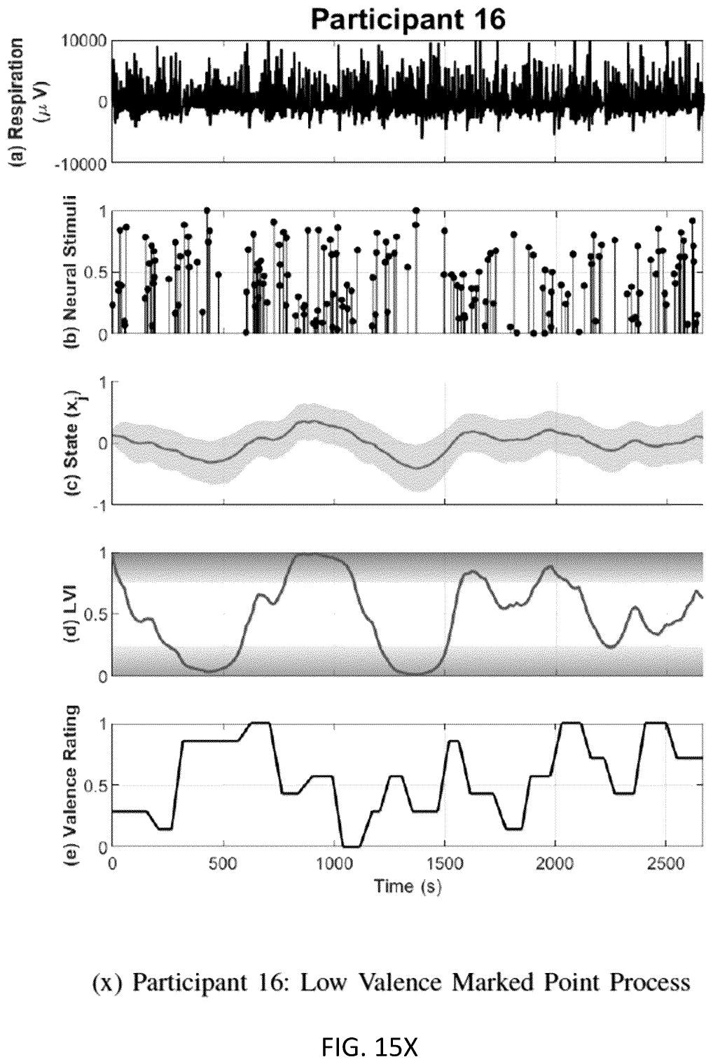

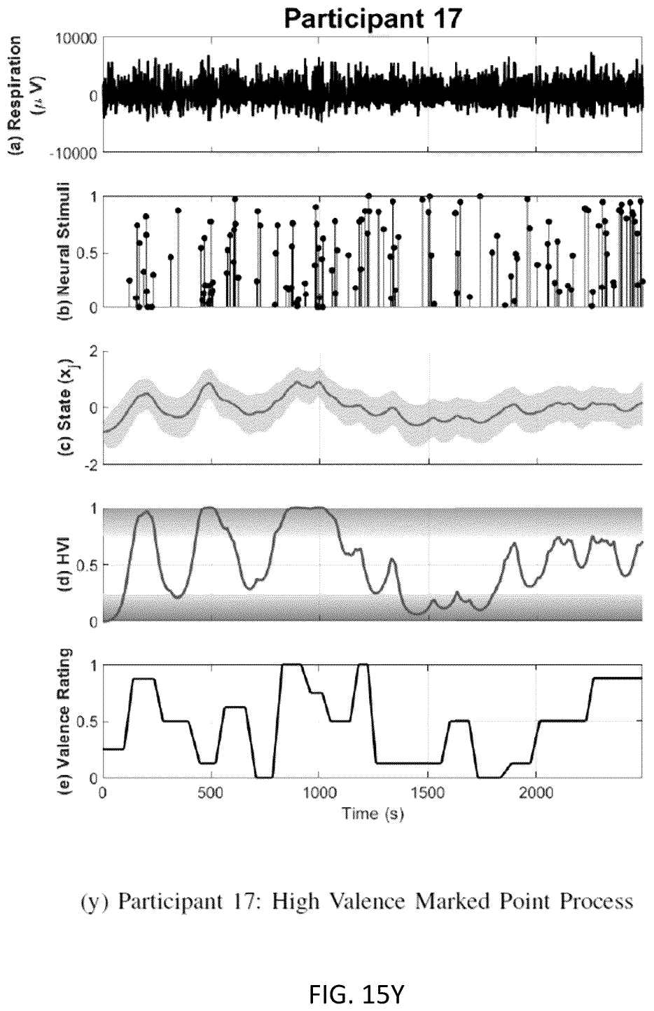

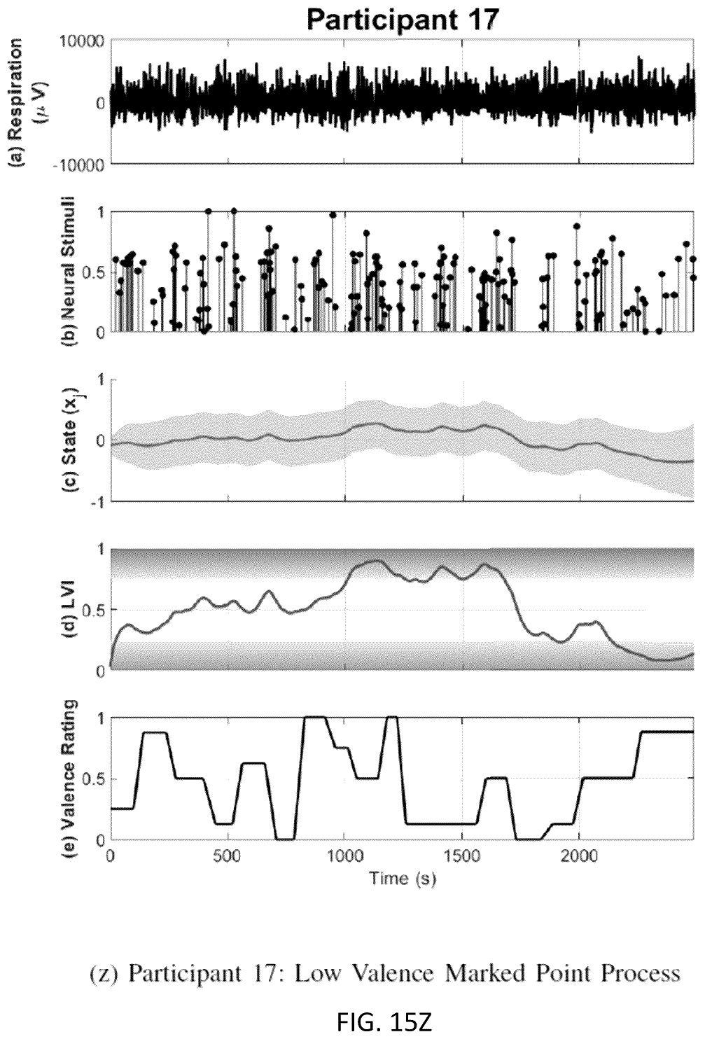

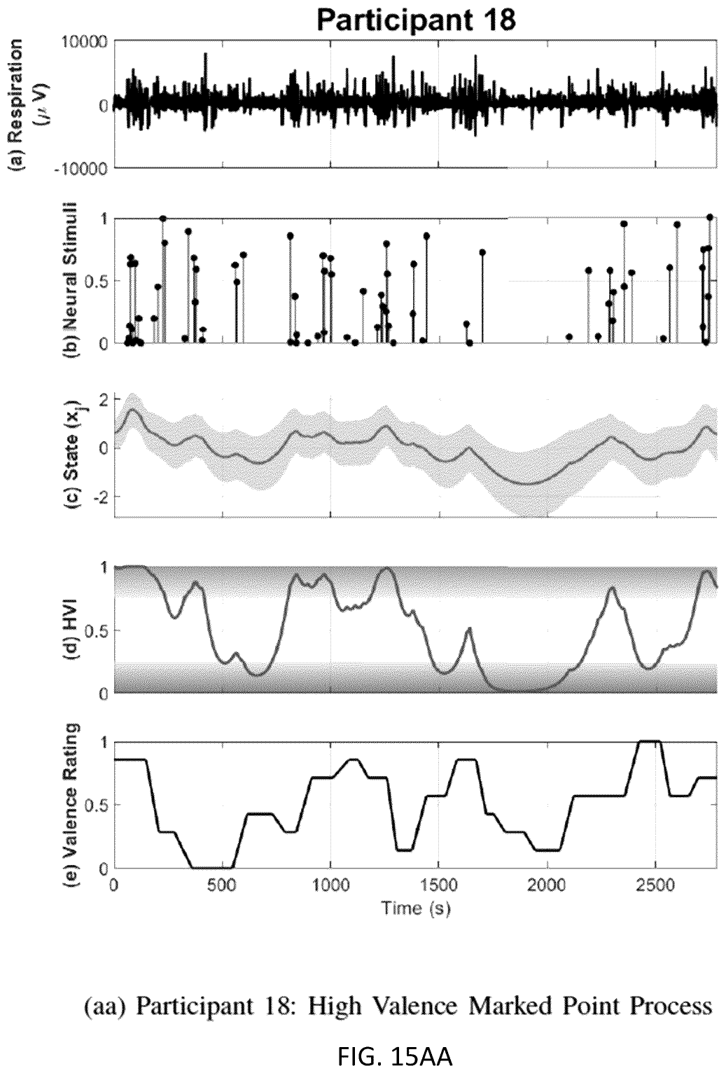

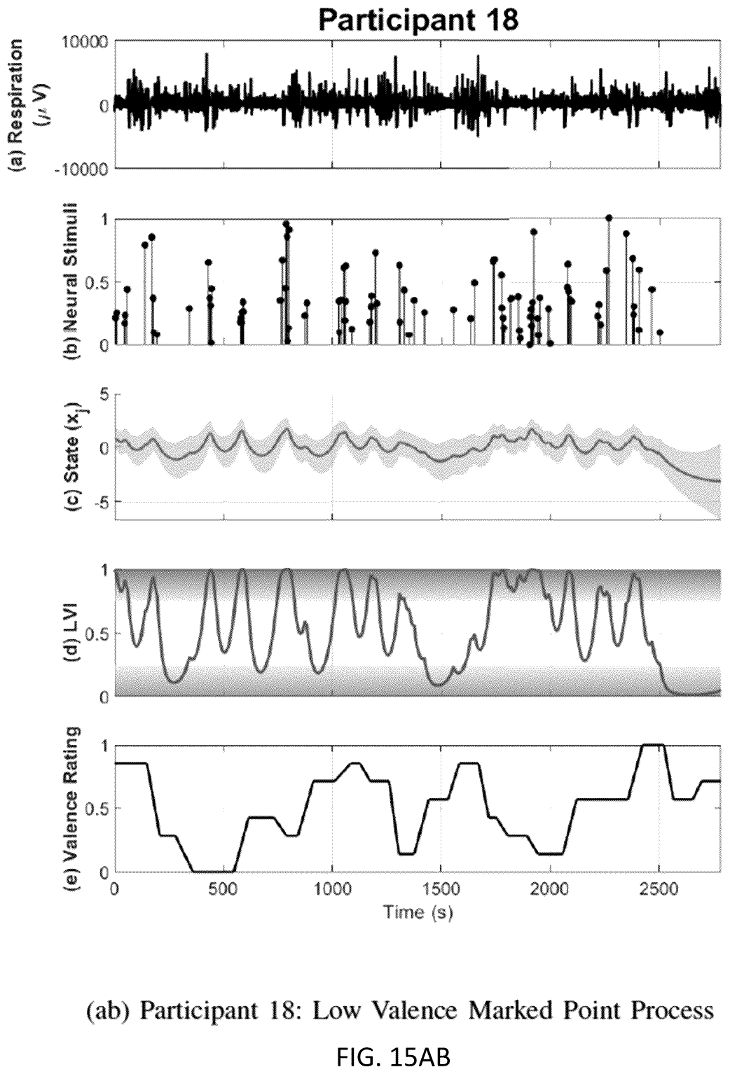

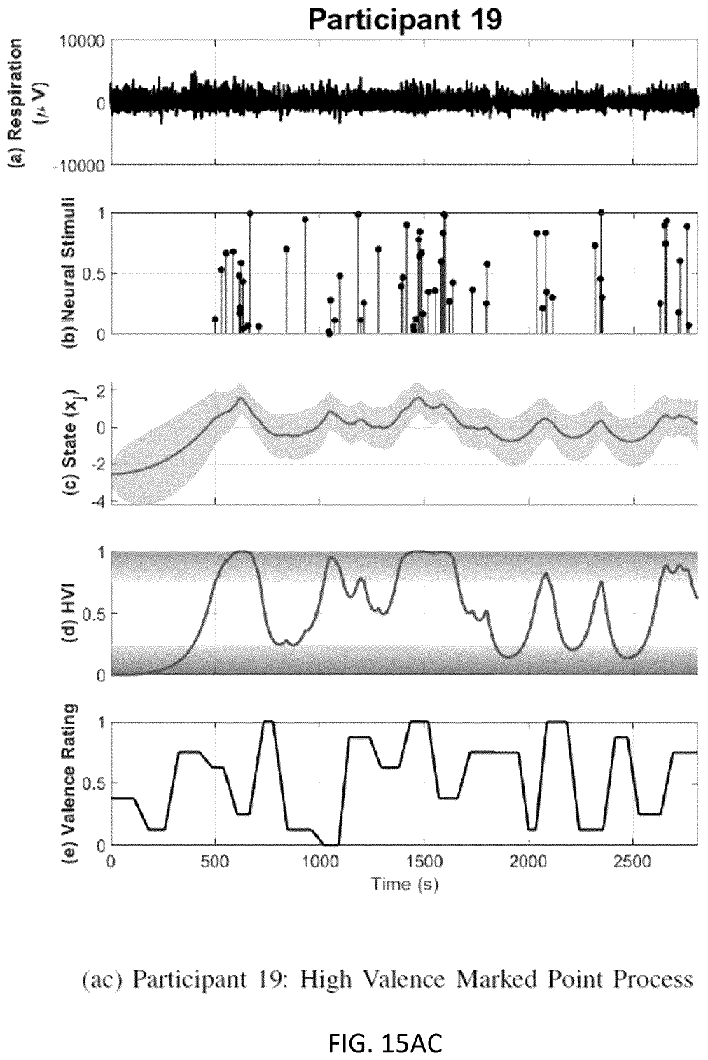

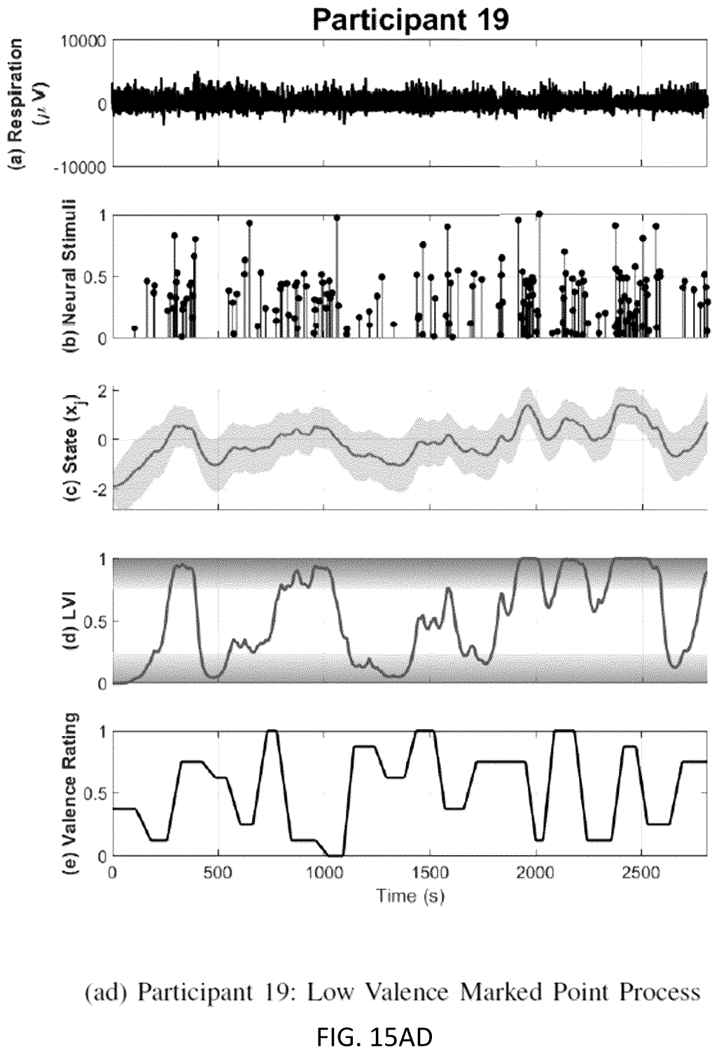

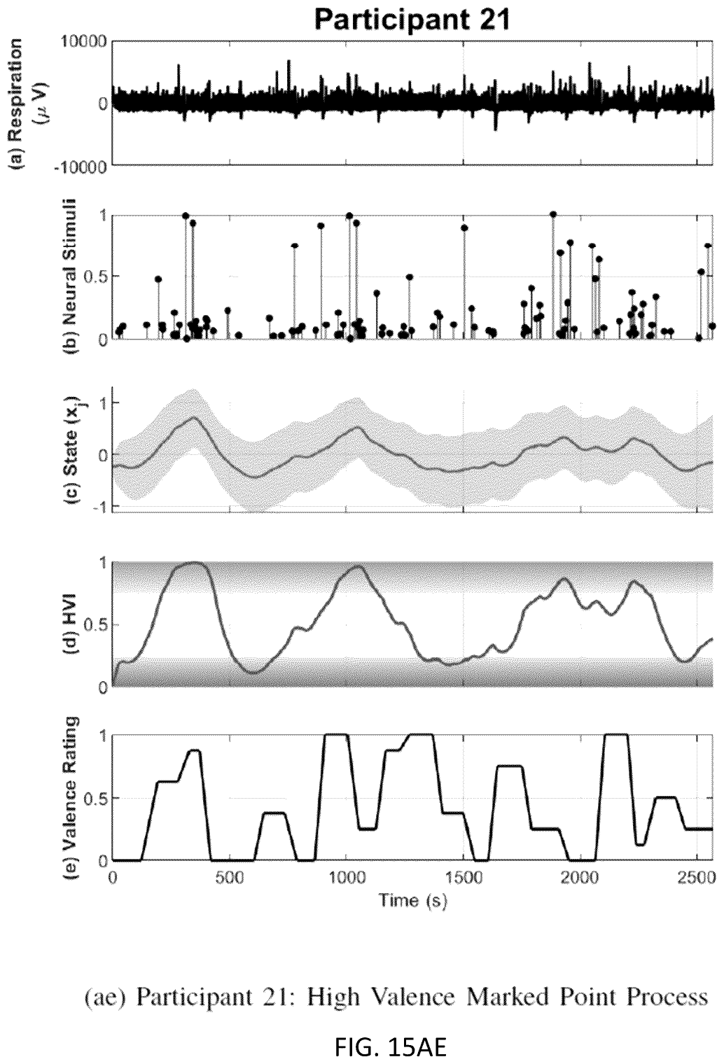

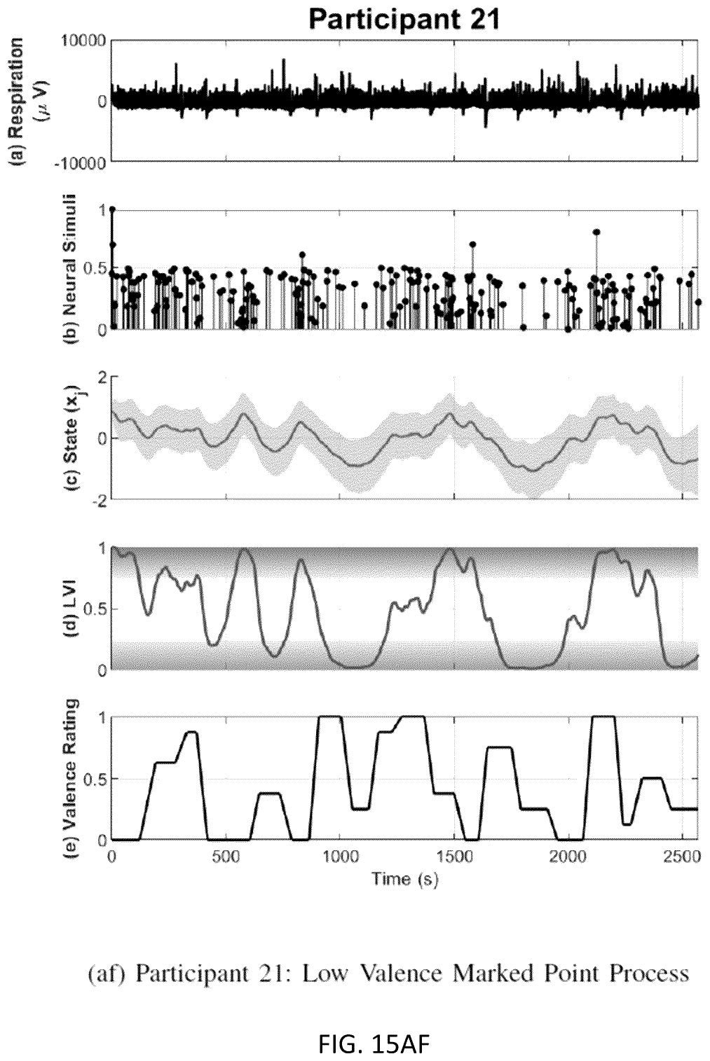

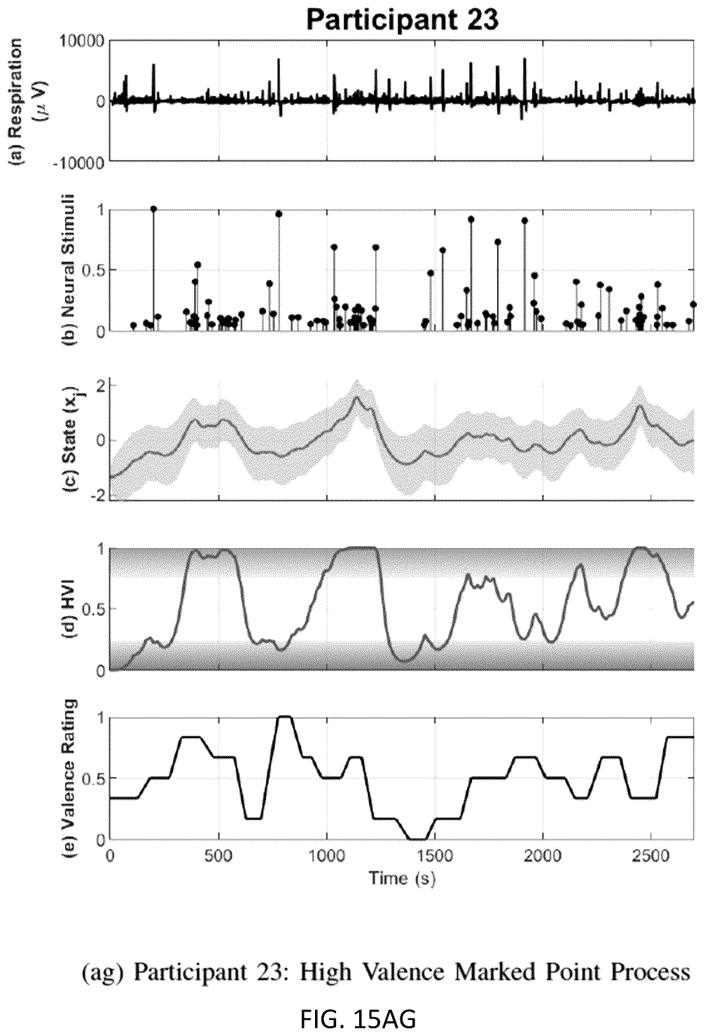

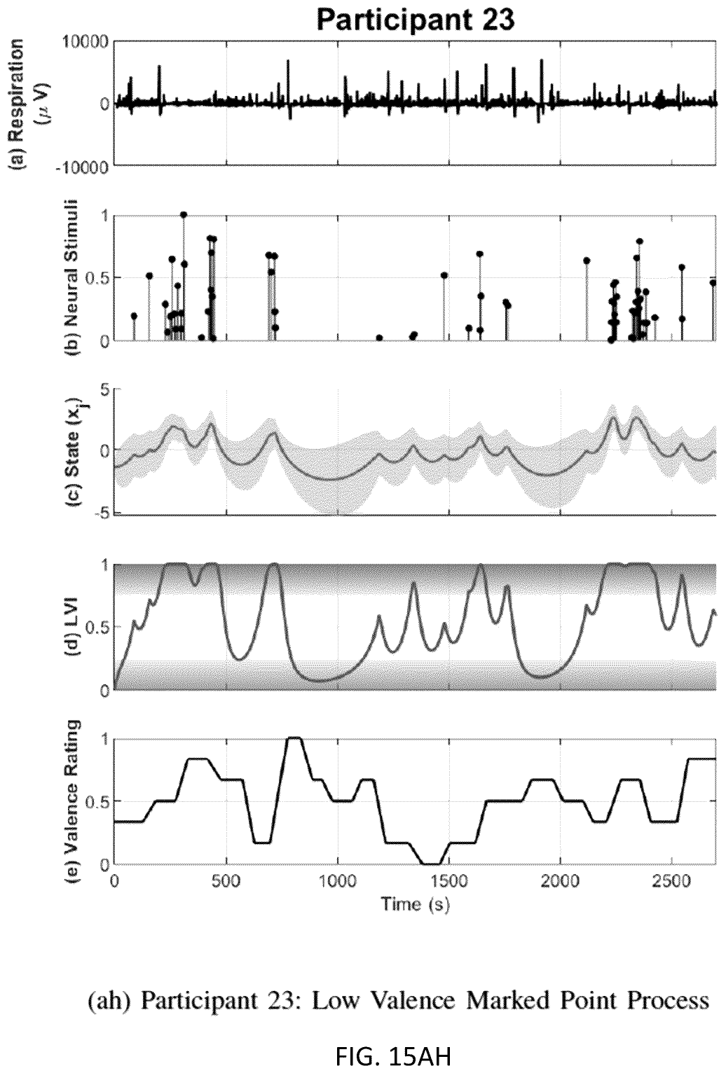

FIGS. 15A-15AH, 16A-16W, and 20A-20H show the generated marked point processes, valence indices, and high and low valence period estimation for participants respectively in the Multimodal Database for Affect Recognition and Implicit Tagging (MAHNOB-HCI). Since the respiration signal is measured via a respiration belt, it is prone to noise and motion artifacting that is apparent throughout the respiration signals shown in FIGS. 15A-15AH. In particular, large amplitude spiking can be observed throughout all signals that have significantly higher amplitudes than typical breaths. In some participants, such as participants 11-14, the mean amplitude of breaths is significantly lower than other participants. In these cases, motion artifacting and noise is more apparent and can significantly impact the quality of estimation. There appears to be high magnitude valence events being recovered surrounding locations of potential motion artifacting, despite the removal of these locations during preprocessing.

In addition, several respiration signals have periods of lower respiration amplitude that could possibly be caused by measurement error rather than a valence response. This behavior can be observed in several participants but is most apparent in the beginning portion of the respiration signal taken from participant 2 and the ending portion of the signal taken from participant 8. These periods result in false identification of low valence periods, since their features (depth of breath and rate of inhalation) are being compared to the mean values taken from the entire signal. To account for these extended deviations in measure values, one could instead only consider recent breaths rather than the entire history of breaths. However, this behavior may be indistinguishable from an extended period of low valence and could reduce the quality of estimation in other participants who have more stable respiration measurements.

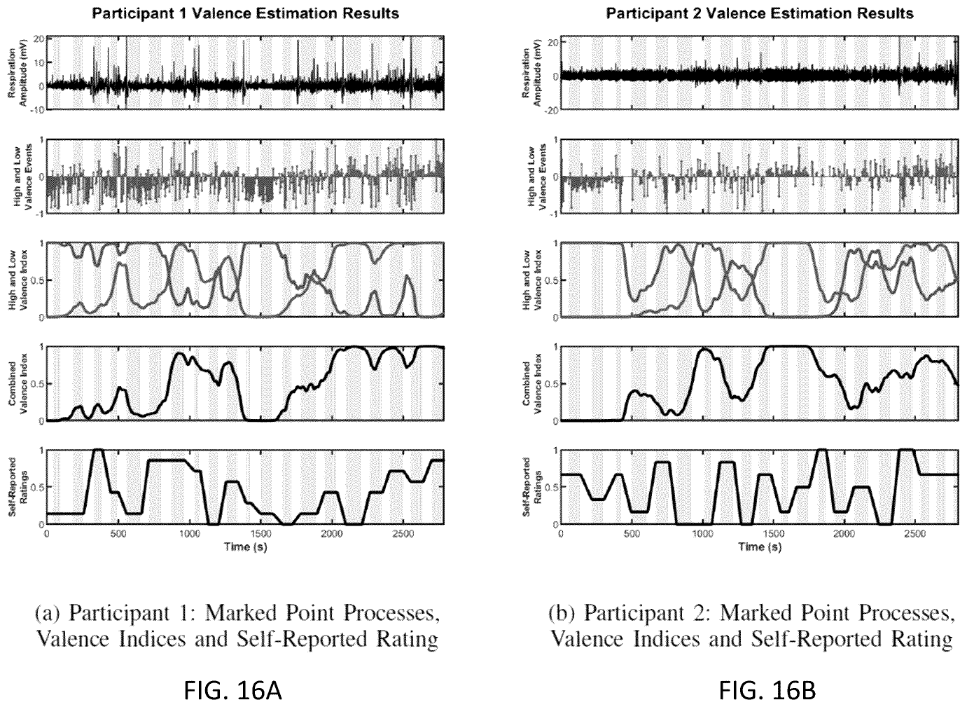

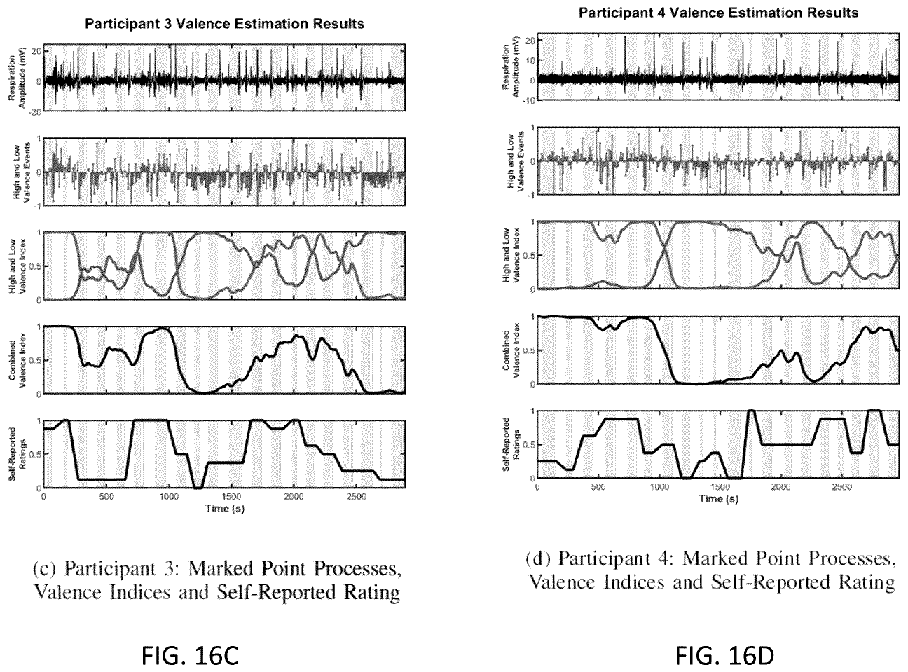

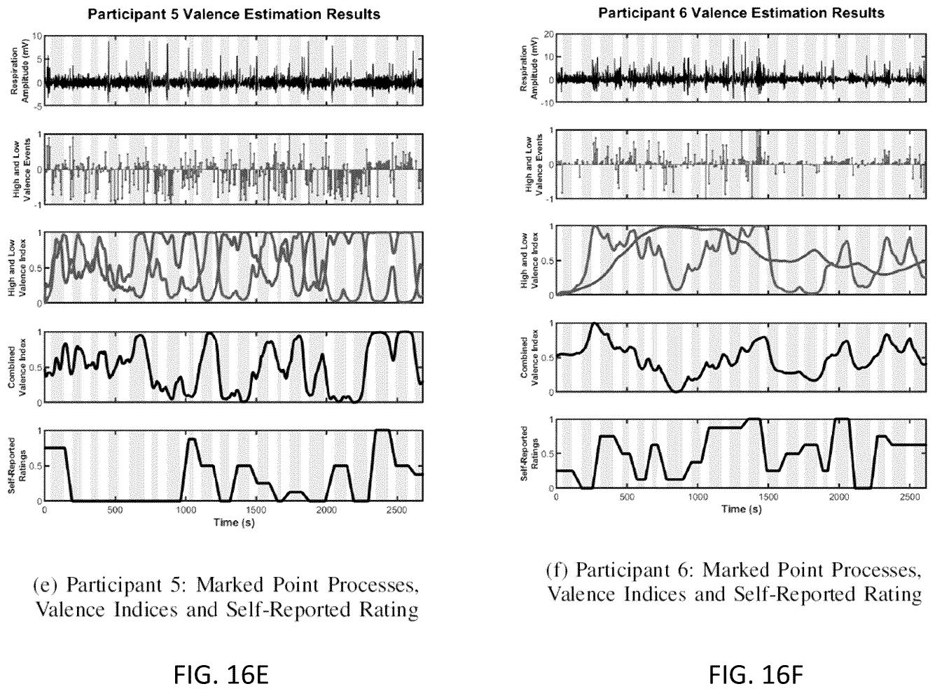

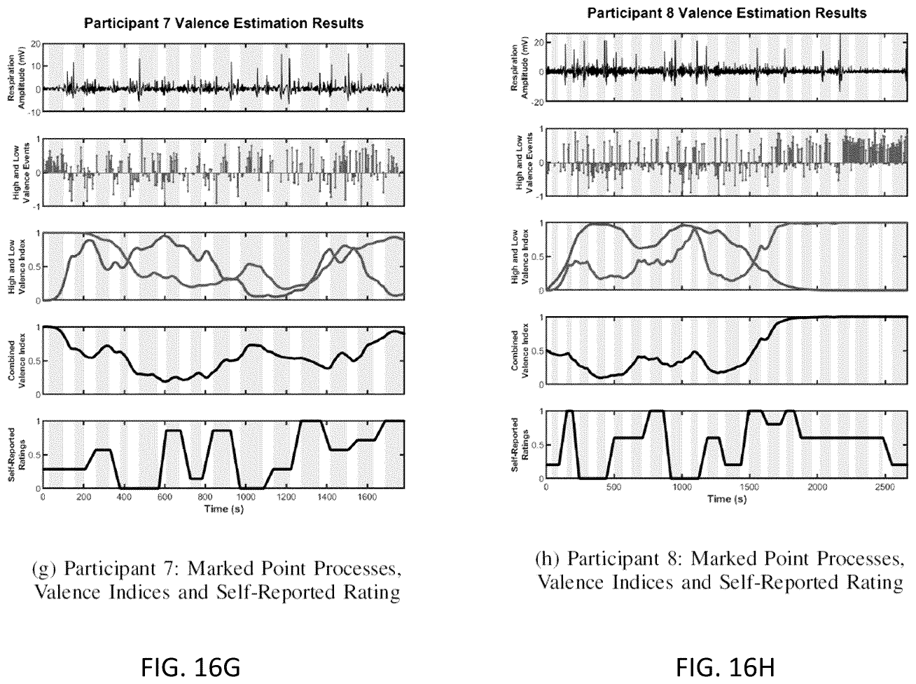

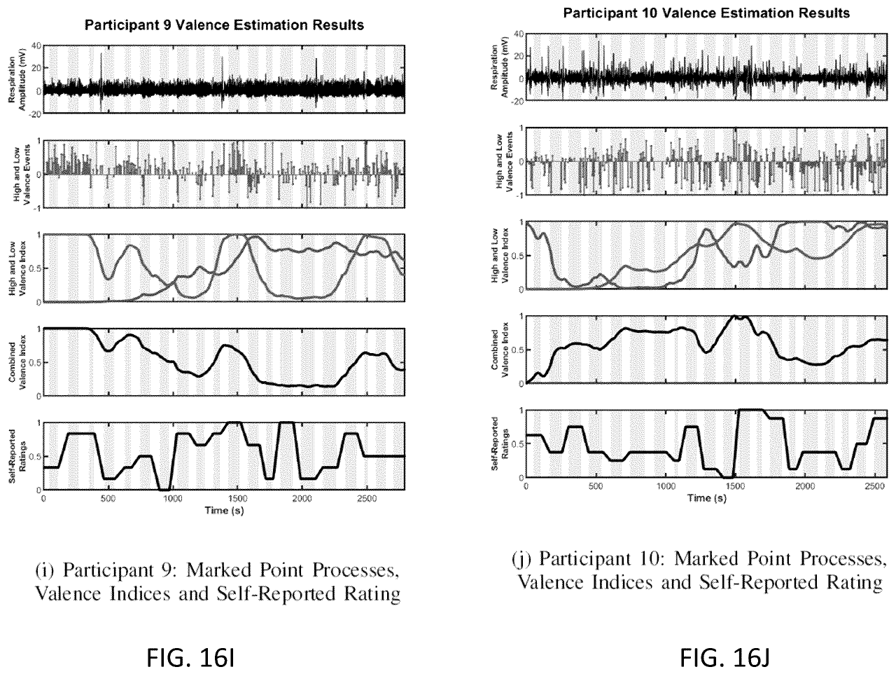

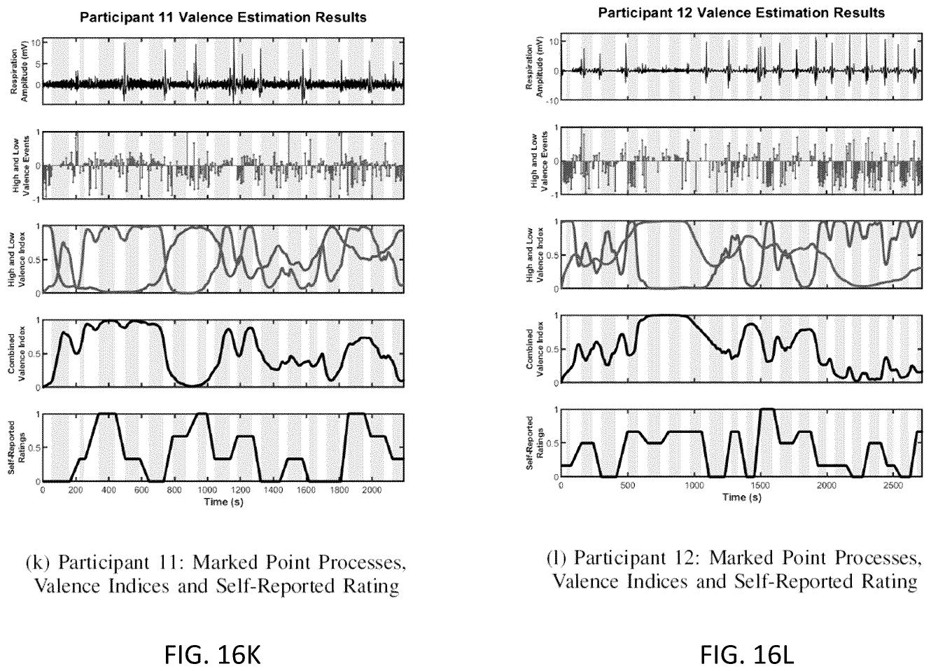

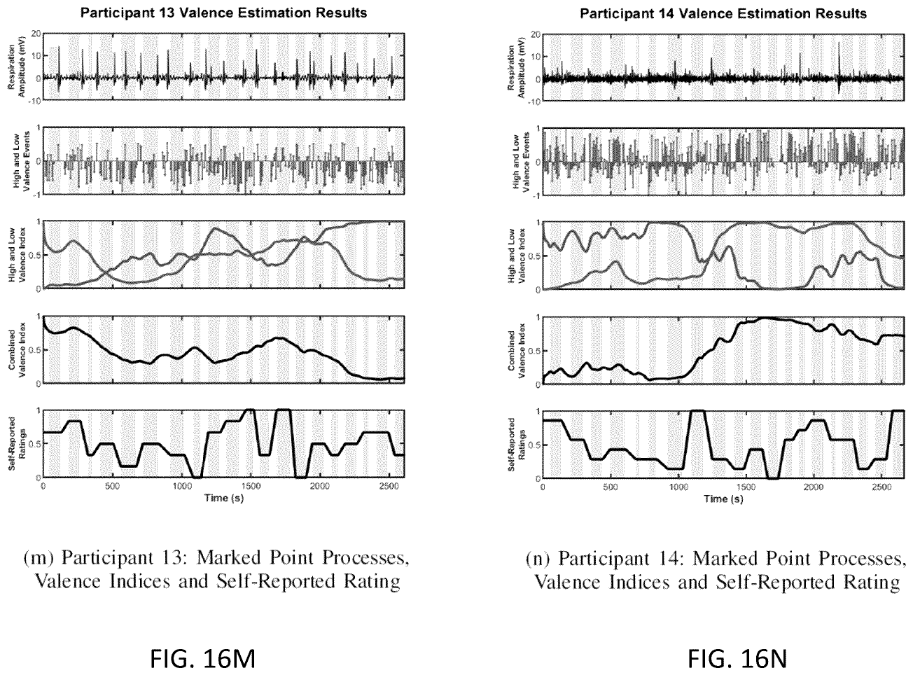

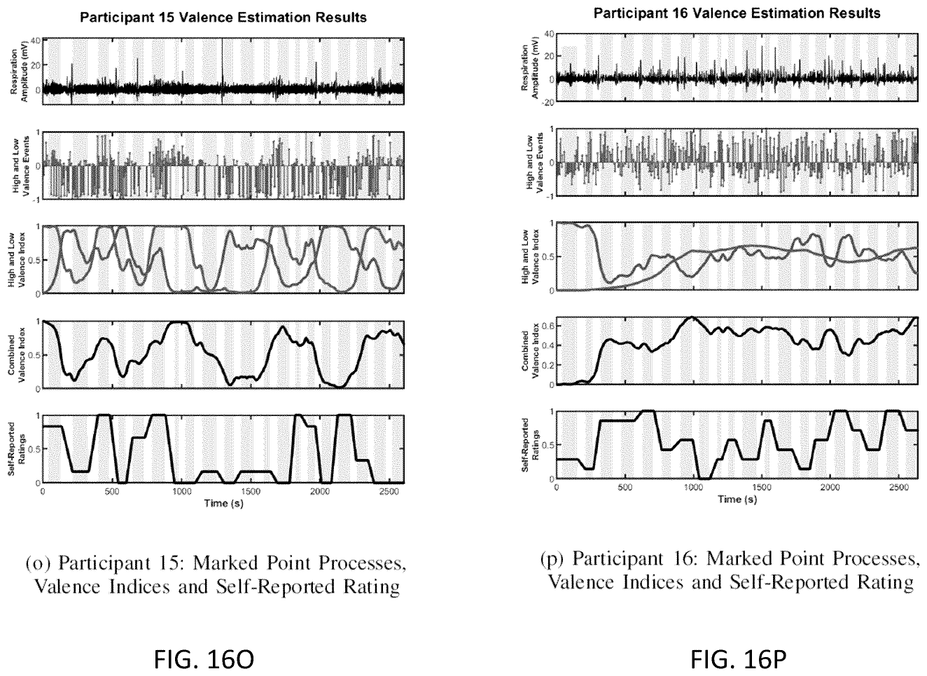

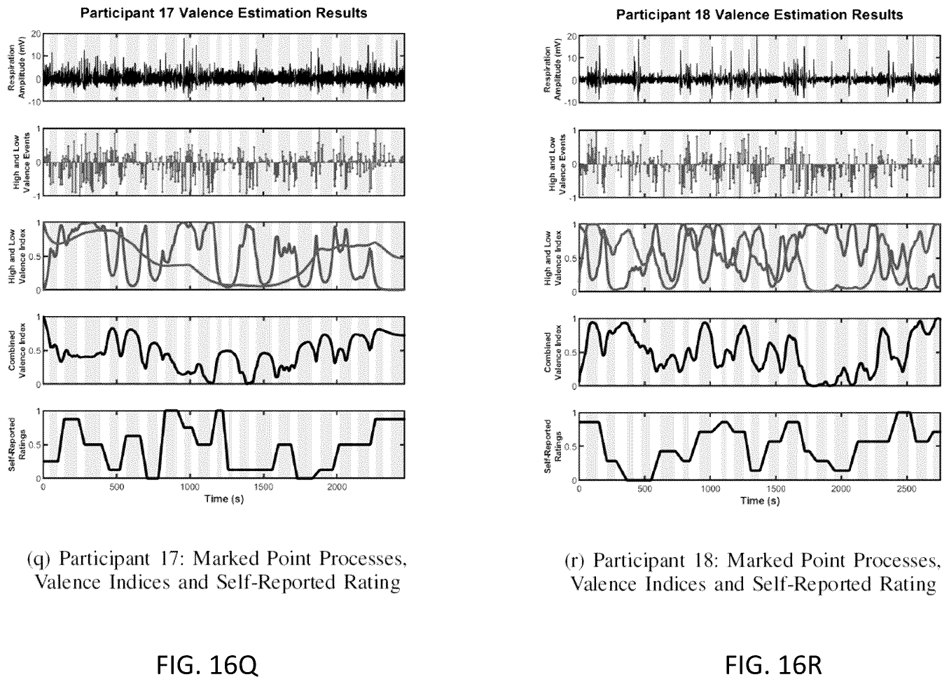

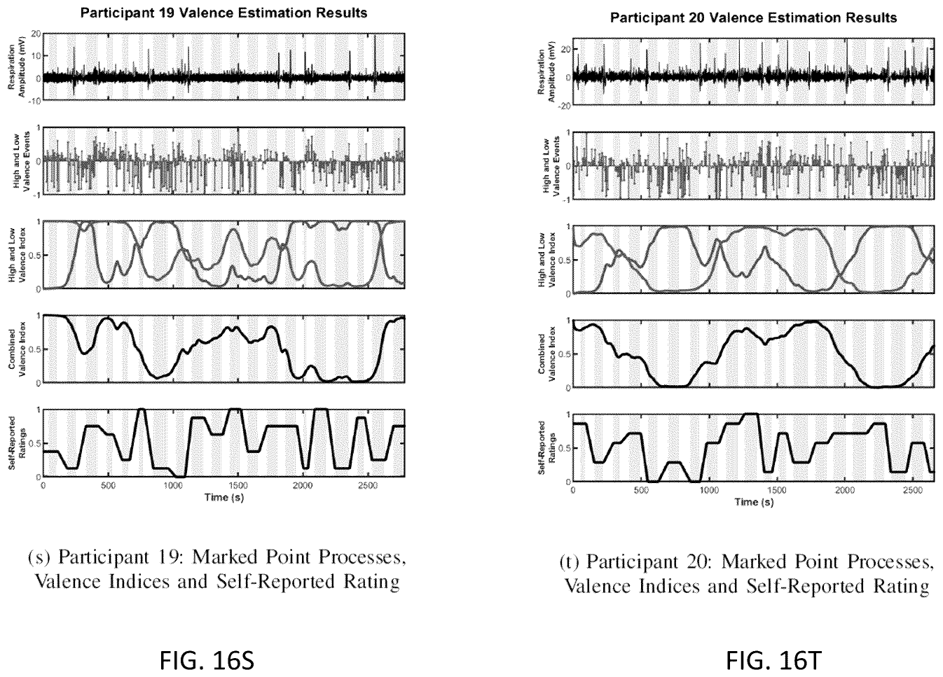

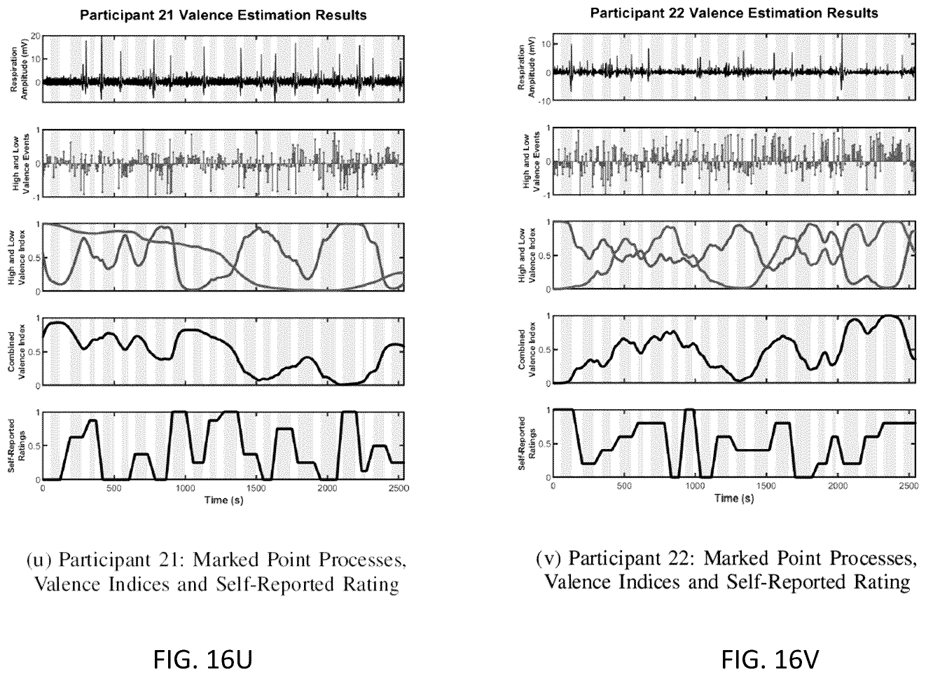

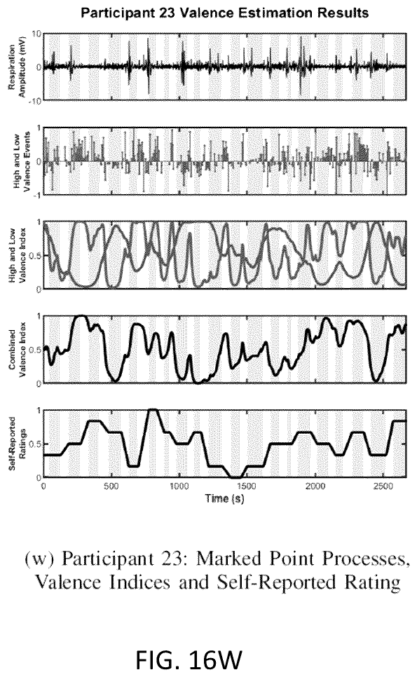

FIGS. 16A-16W show the generation of the marked point process and combination of high and low valence indices for all participants. The low valence index appears to better capture a valence response between trials. As a result, in the combined valence index, the participant was observed returning to neutral valence at a much higher temporal resolution than other participants. Similar behavior can also be observed in other participants, where the low valence index improves the overall estimate by decreasing the estimated valence during extended high valence periods and increasing valence during extended low valence periods. The temporal resolution also appears to be related to the sparsity of the marked point process, which is tuned during the maximization of the negative correlation between the high and low valence events during k-means clustering. In a real-world application, the k-means labeling may need to be verified to prevent the flipping of high and low valence events. Without training, it is difficult to generalize increases or decreases in valence for all subjects, which results in the algorithm failing for some subjects, as noted for potentially 6 participants (2, 7, 9, 17, 19, and 21). In most of these cases, the labeling may be interchanged, or the person may be resistant to physiological changes in response to valence.

In FIGS. 16A-16W, self-reported valence levels are normalized and rescaled for comparison to the combined index. This was done to maintain consistency in the comparisons and identification of high and low valence periods. However, realistically, high and low valence periods could be specific to individuals and dependent on their interpretation of the valence rating since participants are taught to rate their valence levels using a self-assessment manikin, which only provides a vague understanding of the rating scale. After identification of high and low valence periods in the combined valence index and reported valence ratings, statistical analysis was performed by determining if high and low valence events contain the information necessary to distinguish high and low valence periods.

The results in FIGS. 16A-16W show that the model is capable of capturing trends in valence with low temporal resolution. Many of the high and low valence periods are detected in the correct order, but the exact location of the peaks appears to be time shifted in many participants. In addition, the model fails to capture rapid changes in valence and, instead, appears to follow a general trend in valence. This is most apparent in participant 13, where valence estimation shows a period of low valence, a period of high valence, and a period of neutral valence similar to the self-reported ratings, but the rapid trial-to-trial changes that are observed in the ratings are not captured, suggesting the potential use of the model in long term valence monitoring.

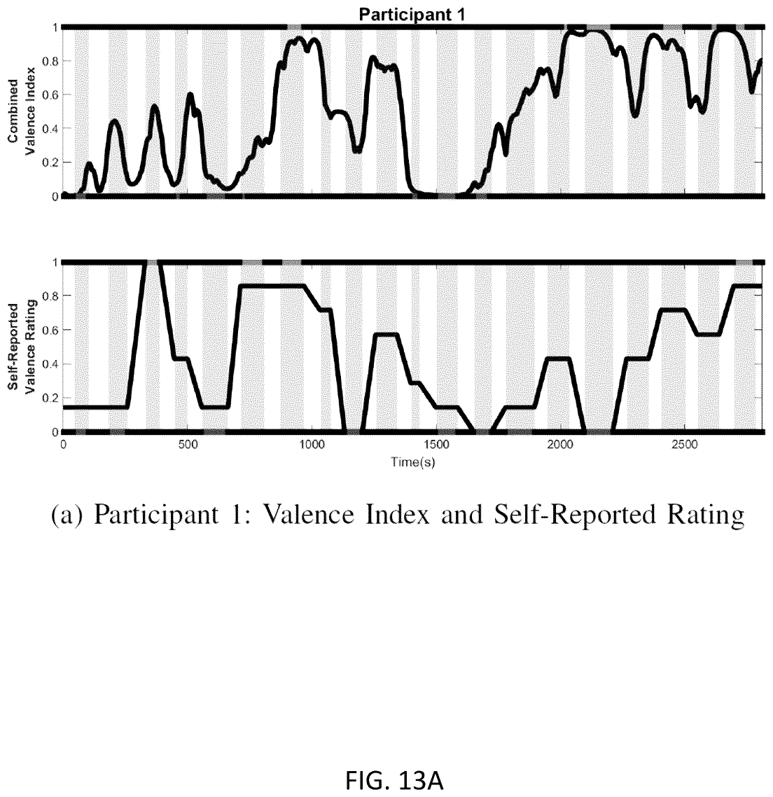

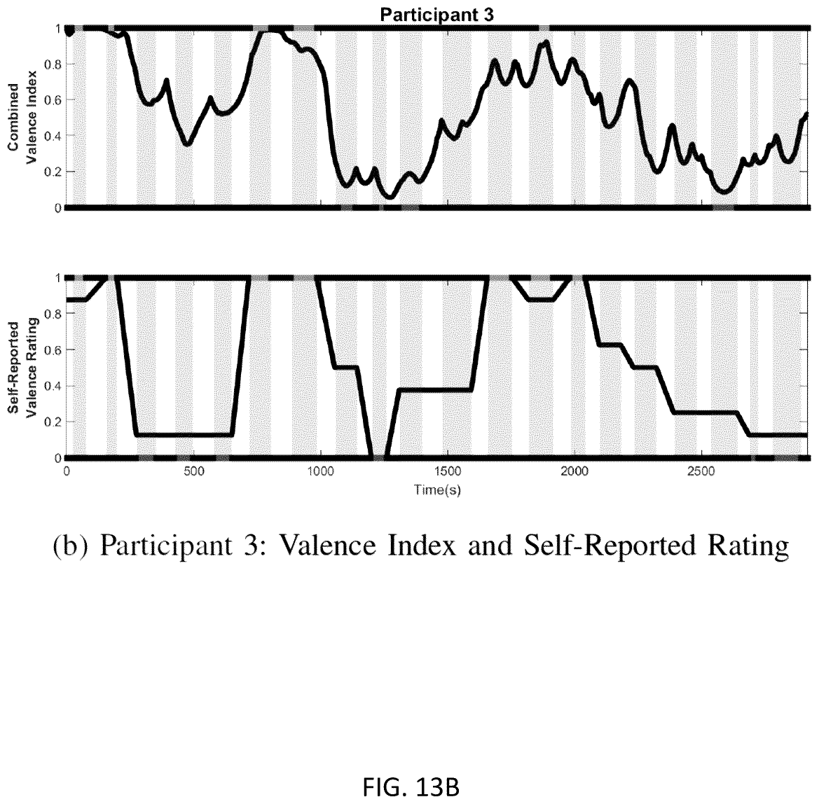

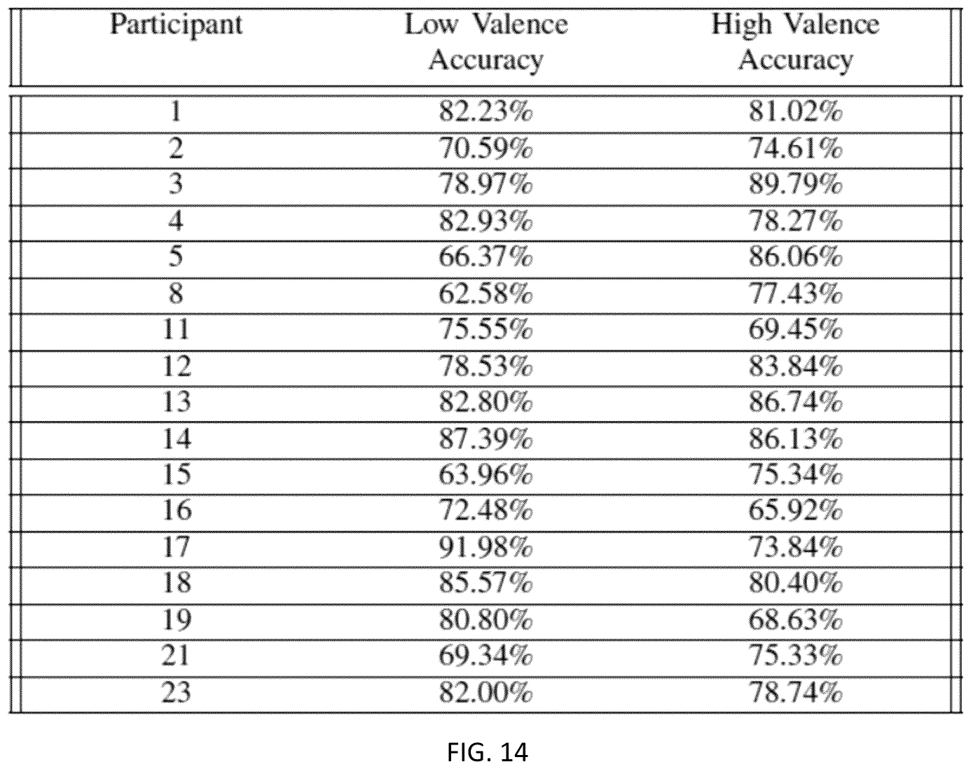

FIG. 13 shows the results for high and low valence period determination based on the estimated valence level. The high and low valence periods identified by the valence index are then compared to the high and low valence periods as defined by the self-reported valence ratings. The valence index appears to fluctuate more than the self-reported valence ratings. Even though a high or low valence period may not be properly identified, changes in valence are still captured by the index. For example, for trials 3-5 of participant 3, low valence is experienced following a period of very high valence. The index captures this change, but the decrease is not significant enough to be considered a low valence period. Instead, it is misidentified as neutral valence. The combined valence index successfully captures trends and changes in valence, but, in some cases, fails to capture the exact magnitude of the change. Accuracy results for all participants are shown in the table of FIG. 14. The mean accuracy over all participants is found to be 78.24% for high valence period estimation and 77.33% for low valence period estimation.

### Discussion

Both the rate of inhalation and depth of breath are important indicators of a valence response in a respiration signal. The high valence index, calculated based on the high valence indicative events associated with increasing rates of inhalation, appears to capture general trends in valence as well as periods of high valence experienced by the participant. Similarly, the low valence index, calculated based on the low valence indicative events associated with decreasing depth of breath, captures periods of low valence but does not appear to capture general trends in valence as well as the high valence index. In many participants, the high valence index alone can identify periods of high and low valence. However, this estimate is improved by the consideration of the low valence index to better identify short-term decreases in valence.

The quality and temporal resolution of the estimated valence index varies greatly between participants. In all participants, the valence index can capture changes in valence between trials but fails to capture the decreases in valence associated with the neutral emotion clips being shown between trials. This suggests that the valence index is better at estimating general trends in valence rather than per-minute changes. The temporal resolution of each participant appears to be unique and dependent on the probability of valence events occurring throughout the experiment. For participants with poor temporal resolution, as in the valence index fails to capture some trial-to-trial changes in valence, their associated marked point processes contain fewer total pulses, suggesting that these participants could be less susceptible to changes in respiration as a result of an emotional response. In participants with better temporal resolution, there is a larger number of total valence events detected. In these participants, the predicted valence index has improved accuracy, particularly in detecting periods of very high valence. In many participants, the location of high valence periods appears to be shifted later in time. This could be due to the exact location of the high valence event being unknown within the clip viewing, as well as respiration activity gradually recovering to normal after a period of high valence. In one participant, the low valence index appears to be an indicator of high valence levels, suggesting that this participant could be having an opposite physiological response (shallow breaths during high valence) unlike other participants. An improved model could determine the individual's natural physiological response during parameter estimation to consider outliers such as this participant.

The k-means clustering algorithm for event detection offers several advantages from the previous valence estimation method presented in (Shenechi at al. 2017). The k-means method is also more robust to variations between participants without the need for training. The previous model utilized training to find subject specific thresholds for each of the features being assessed. Though this can lead to better results, training can be reliant on the quality of self-reported ratings and requires additional user input in order to function. In addition, supervised learning methods may be prone to overfitting valence estimation to individuals and specific stimuli rather than a generalized physiology-based method. In addition to the training, the previous method did not consider the amplitude of the event in its estimation, which appears to contain more information for low valence estimation according to FIGS. 18A-18B. The amplitude information can be important in distinguishing more significant events. Particularly, during the removal of excessive pulses in the MPP, the smallest magnitude pulses are removed that are more likely to correspond to neutral valence. On the other hand, the very large amplitude events are more likely to be associated with an emotional response.

During the k-means grouping of individual breaths, two important steps are taken to drastically improve the quality of the estimate and reduce misidentification of breaths: normal breaths are removed and the negative correlation between the high and low valence MPP is maximized over several iterations. This method may utilize the heuristic nature of removing normal breaths and improving the k-means algorithm's grouping of high and low valence breaths. Static probability thresholds of 0.2 for both rate of inhalation and depth of breath are able to capture trends in valence for most participants. In some participants, these static thresholds are not optimal to capture the necessary amount of valence events for estimation resulting in poorer estimates or possible convergence issues. However, the estimate is significantly improved by adjusting the thresholds for each participant depending on their individual valence responses. For all participants, the optimal thresholds are between 0.2 and 0.4 which could be determined by minimizing the error between the estimated valence and reported valence ratings. Participants with lower thresholds are likely more susceptible to changes in their respiration pattern resulting from emotion. In other words, during a change in valence, these participants show larger changes in inhalation rate or depth of breath. Participants with higher probability thresholds are likely less susceptible to changes in respiration pattern, meaning they show smaller changes in their respiration pattern in response to a change in valence. This also corresponds to poorer quality of estimation for these participants, suggesting that the accuracy of the model can vary drastically between people dependent on their individual susceptibility to a physiological response. This susceptibility may not be specific to respiration and could encompass other signals such as GSR and ECG.

Several aspects of this model are specific to the experimental setup from the MAHNOB-HCI database. In particular, the preprocessing and artifact detection are highly specific to the recordings in this database. The cutoff frequencies of 0.05 Hz and 4 Hz could be applied to any respiration signal but have been optimized for these recording conditions. In real-world applications, the respiration signal could be exposed to much more significant noise and artifacting that could make feature extraction and eventually valence state estimation more difficult. The preprocessing and motion artifact detection would have to be adjusted and improved significantly for practical applications of valence estimation. In addition to the preprocessing, the window size of peak detection would also have to be adjusted depending on the feasible respiration rate for specific tasks. In the disclosed study, participants are viewing video clips in a controlled setting; so, responses to emotional stimuli may not be as significant compared to someone experiencing an extreme emotion in their daily life or for those suffering from mental disorders. For these cases, the window size would have to be reduced from 1 second to detect extremely fast breathing that could be expected to occur.

The accuracy of the state-space model in predicting high and low valence periods indicates the efficacy of the model as a valence monitor. For example, one potential application could be to monitor patients suffering from certain mental disorders. People suffering from depression could experience extended periods of low valence episodes characterized by periods of shallow breathing, which would be apparent in the low valence index calculated by the model. In addition, the model could also be used for multimedia recommendation applications. For example, high valence periods can be used to quantify a person's interest when viewing media without the need of self-reported user feedback. In both cases, capturing the general trends in valence with lower temporal resolution would suffice since the observed valence periods would persist for extended periods. For a practical implementation of the model, self-reported valence ratings have to be initially provided for threshold determination. Marked point process generation and valence index calculations would have to be performed in batches to compensate for the backward filtering as required by the expectation-maximization algorithm.

Recent developments in wearable technology and the ability to recover respiratory information from PPG signals (Roy et al. 2021), makes respiration practical for continuous monitoring. Unlike physiological signals such as electroencephalogram (EEG) and electromyogram (EMG), the devices capable of recording respiratory activity are already practical and measurable with smartwatch-like wearable devices. Valence estimation can be performed using known inhalation rates and amplitudes derived from PPG signals, which can be collected via wrist worn wearables. Estimation can be performed in near-real time based on a history of respiration activity, since the expectation maximization algorithm prevents the real-time use of the estimation as it requires backward filtering (De Fazio et al. 2021). The valence estimation can be easily implemented in many of these devices to provide feedback to both healthy users and monitor at risk users when necessary. Respiration also differs from other physiological signals in that it can be self-regulated. Manually controlling respiration following unpleasant emotions has been shown to reduce the psychological impact of the emotion and return the person to neutral valence levels. The model can include a control using respiration to correct periods of low valence. The valence estimation would alert the user when they are experiencing extended periods of low valence and signal them to correct their breathing.

### Further Results and Discussion

FIGS. 15A-15AH, 16A-16W, and 17A-17Q show the generated marked point processes, valence indices, and high and low valence period estimation for all participants respectively in the Multimodal Database for Affect Recognition and Implicit Tagging (MAHNOB-HCI). Since the respiration signal is measured via a respiration belt, it is prone to noise and motion artifacting that is apparent throughout the respiration signals shown in FIGS. 15A-15AH. In particular, large amplitude spiking can be observed throughout all signals that have significantly higher amplitudes than typical breaths. In some participants, such as participants 11-14, the mean amplitude of breaths is significantly lower than other participants. In these cases, motion artifacting and noise is more apparent and can significantly impact the quality of estimation. There appears to be high magnitude valence events being recovered surrounding locations of potential motion artifacting, despite the removal of these locations during preprocessing.

In addition, several respiration signals have periods of lower respiration amplitude that could possibly be caused by measurement error rather than a valence response. This behavior can be observed in several participants but is most apparent in the beginning portion of the respiration signal taken from participant 2 and the ending portion of the signal taken from participant 8. These periods result in false identification of low valence periods, since their features (depth of breath and rate of inhalation) are being compared to the mean values taken from the entire signal. To account for these extended deviations in measure values, one could instead only consider recent breaths rather than the entire history of breaths. However, this behavior may be indistinguishable from an extended period of low valence and could reduce the quality of estimation in other participants who have more stable respiration measurements.

FIGS. 16A-16W shows the combination of high and low valence indices for all participants. The low valence index appears to better capture a valence response between trials. As a result, in the combined valence index, the participant was observed returning to neutral valence at a much higher temporal resolution than other participants. Similar behavior can also be observed in other participants, where the low valence index improves the overall estimate by decreasing the estimated valence during extended high valence periods and increasing valence during extended low valence periods. The temporal resolution also appears to be related to the sparsity of the marked point process, which is tuned during the threshold determination step. The threshold determination step involves minimizing mean-squared error between the combined valence index and self-reported valence ratings for 25% of the recording. These thresholds are then used to optimize the valence event identification and improve estimated valence. In a real-world application, this could be performed once per user and requires approximately ten minutes of computation time.

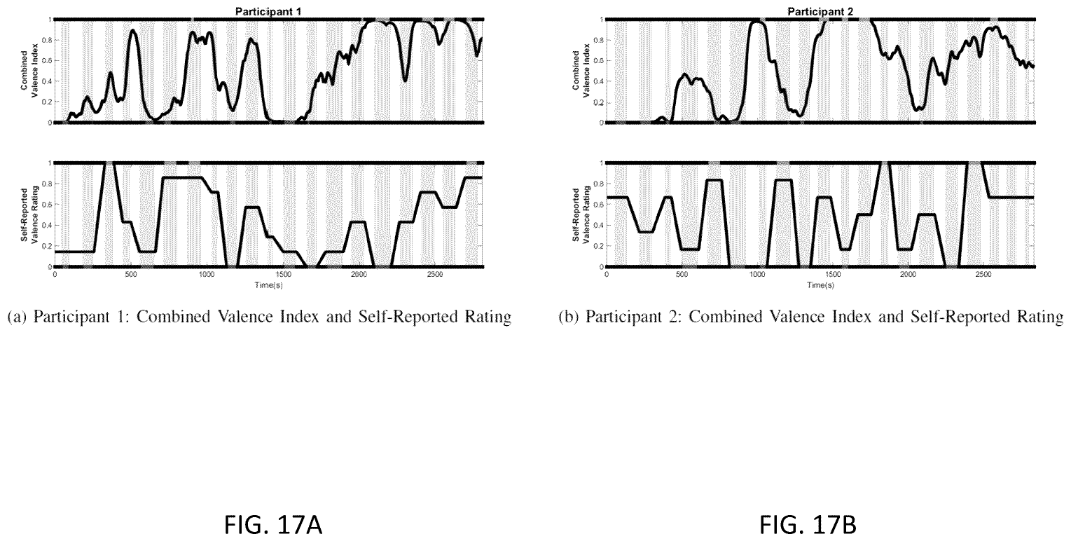

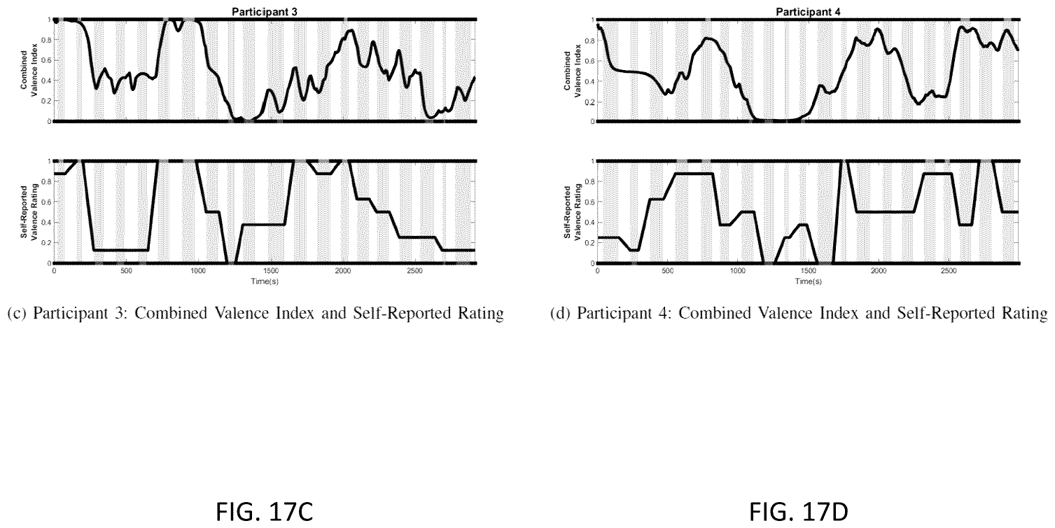

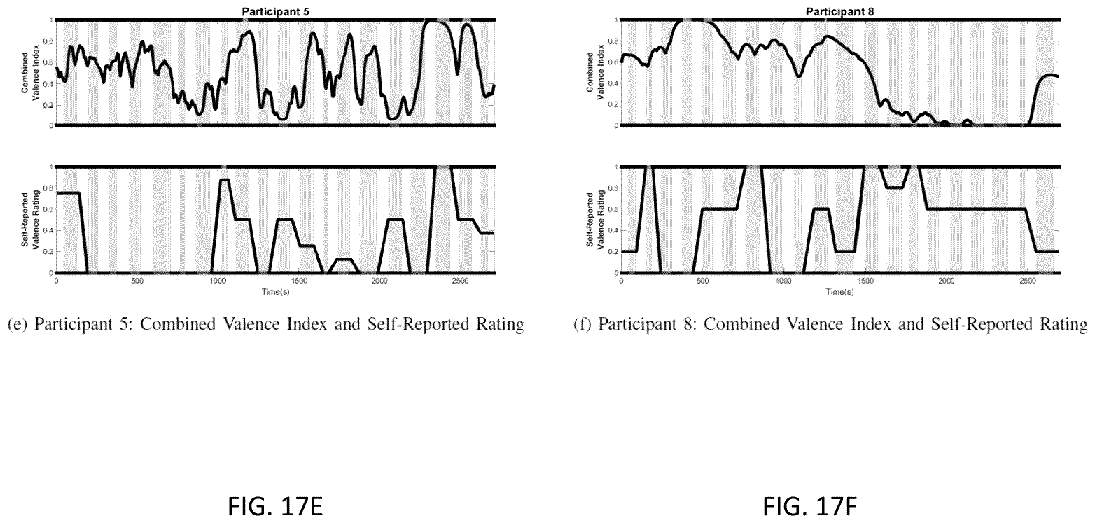

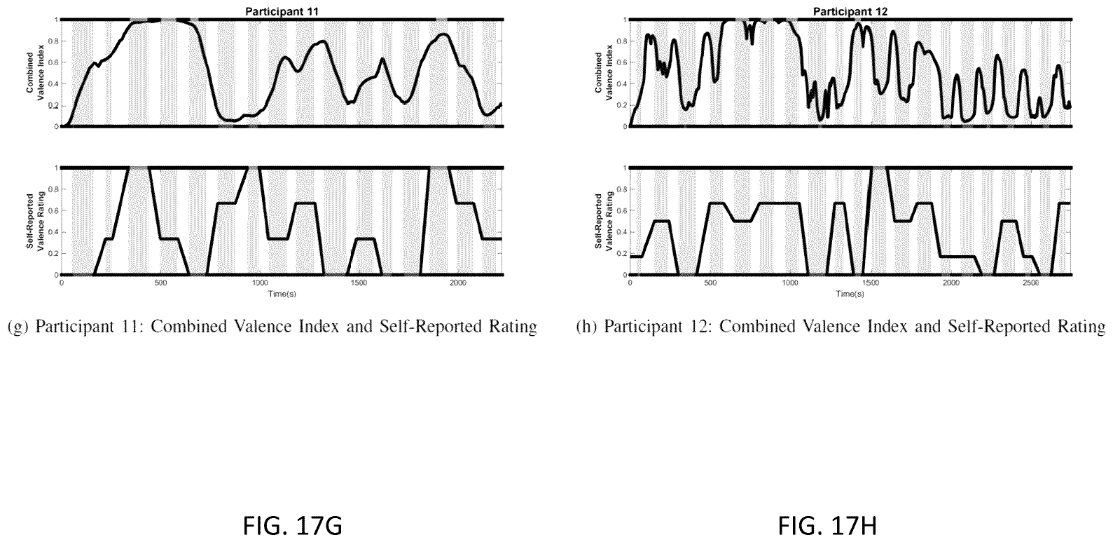

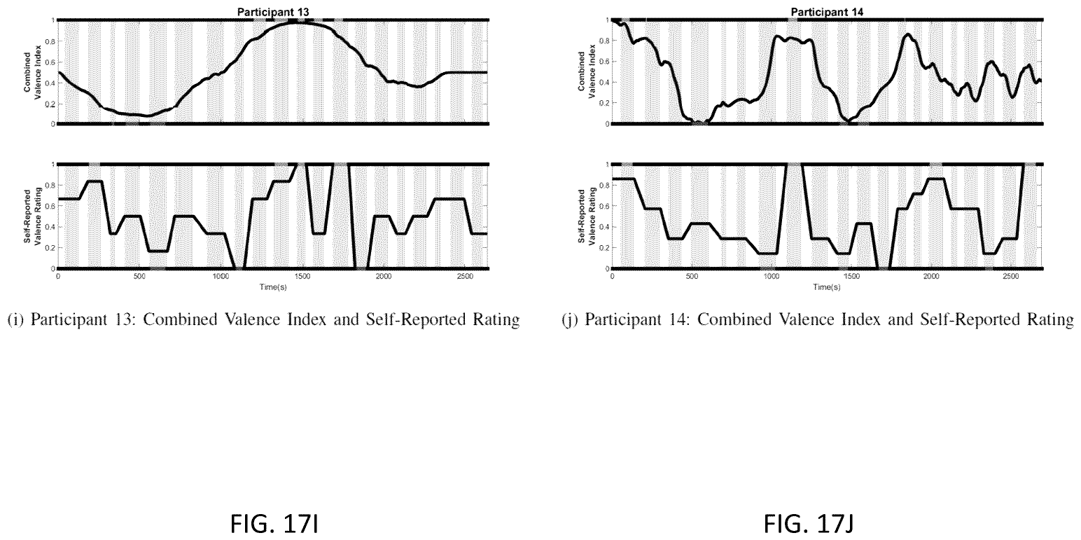

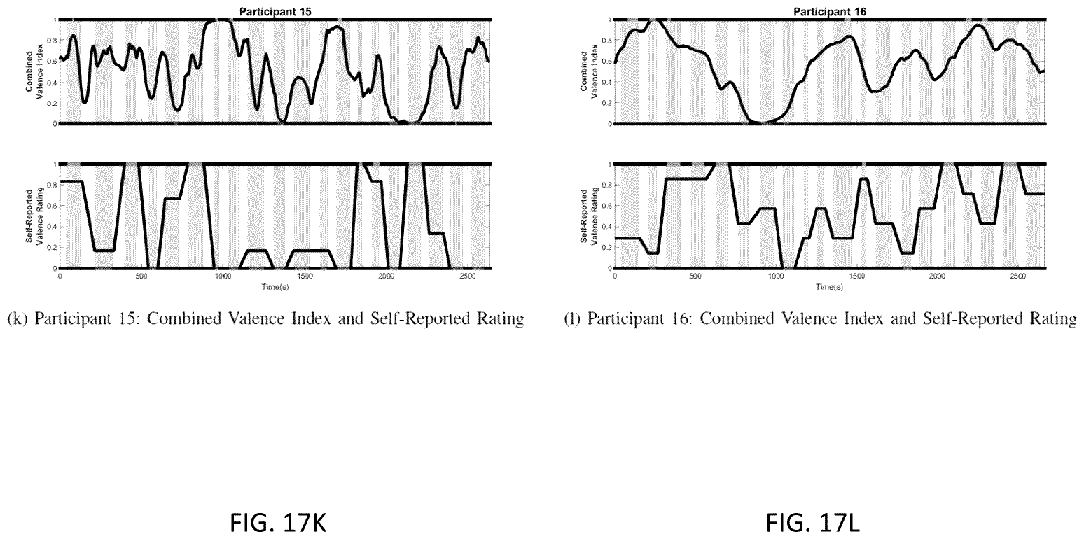

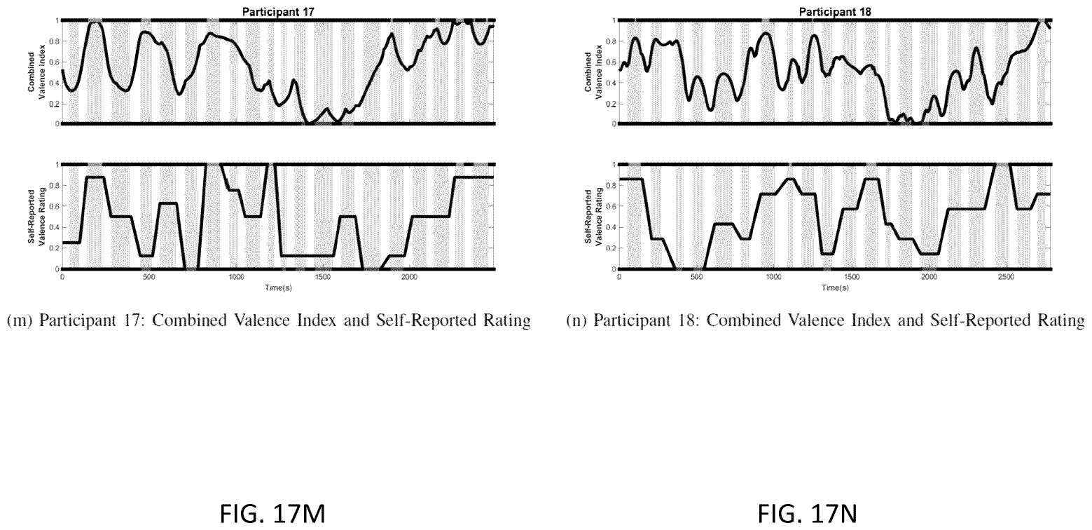

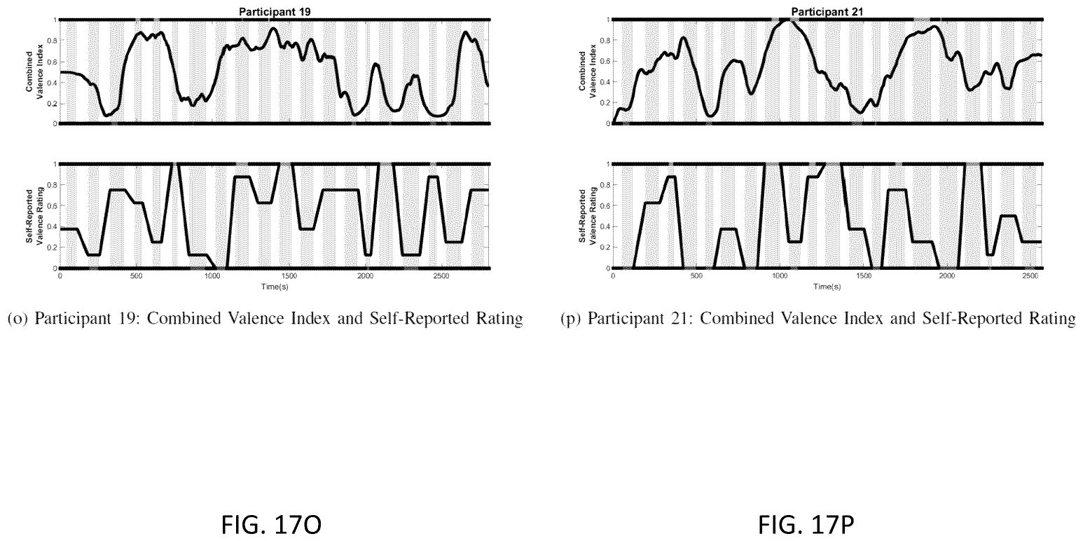

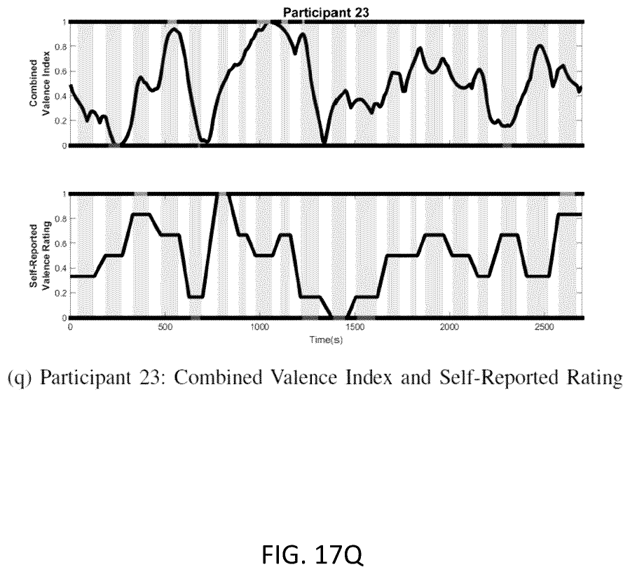

In FIGS. 17A-17Q, self-reported valence levels are normalized and rescaled for comparison to the combined index. This was done to maintain consistency in the comparisons and identification of high and low valence periods. However, realistically, high and low valence periods could be specific to individuals and dependent on their interpretation of the valence rating since participants are taught to rate their valence levels using a self-assessment manikin, which only provides a vague understanding of the rating scale. After identification of high and low valence periods in the combined valence index and reported valence ratings, accuracy calculations were performed by determining if a high or low valence event was properly detected for each time bin of 2 seconds. Since the presence of high or low valence periods is rare relative to neutral valence periods, the calculated accuracy and specificity are relatively large, while the sensitivity remains low. This is a result of the thresholds used to define high and low periods in the combined valence index being stricter than the valence ratings, since one goal was to know with high certainty if the participant is undergoing a high valence period.

The results show that the model is capable of capturing trends in valence with low temporal resolution. Many of the high and low valence periods are detected in the correct order, but the exact location of the peaks appears to be time shifted in many participants. In addition, the model fails to capture rapid changes in valence and, instead, appears to follow a general trend in valence. This is most apparent in participant 13, where valence estimation shows a period of low valence, a period of high valence, and a period of neutral valence similar to the self-reported ratings, but the rapid trial-to-trial changes that are observed in the ratings are not captured, suggesting the potential use of the model in long term valence monitoring.

### Conclusions

In this study, a method to track valence based on changes in respiration pattern was presented. It was found that variations in respiratory pattern could be used to quantify and identify high and low valence periods during emotion elicitation. Using the k-means clustering algorithm, the model groups individual breaths based on extracted features, which it then uses to infer the presence of high or low valence events relating to changes in valence, predict a high and low valence state, then combine the two states into a combined, improved valence estimate. The estimation cannot detect minute-to-minute changes in valence but succeeds in capturing general trends in valence throughout video clip viewings.

The valence estimation can have applications in continuous monitoring of valence such as multimedia recommendation and long-term monitoring of patients suffering from mental disorders who may be experiencing extended periods of low valence. Particularly, improvements and accessibility to wearable sensors capable of measuring respiration makes a respiration-based valence estimator an invaluable tool in emotion recognition.

### Computing Environment

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

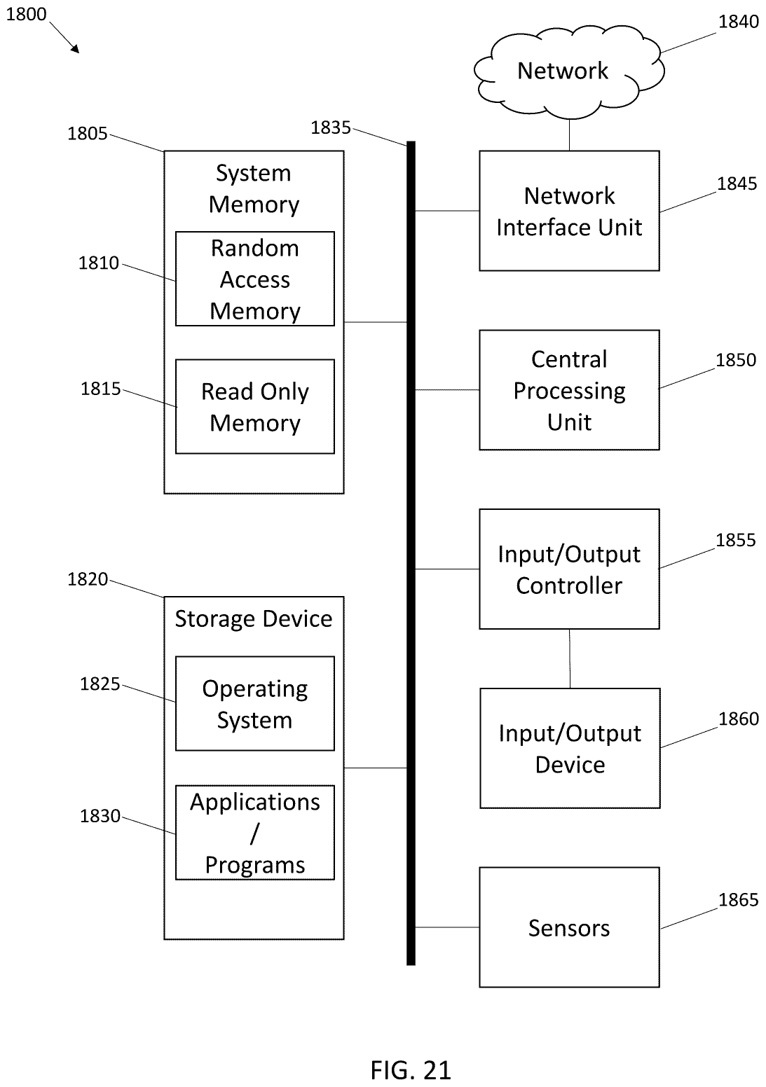

FIG. 21 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 21 depicts an illustrative computer architecture for a computer 1800 for practicing the various embodiments of the invention. The computer architecture shown in FIG. 21 illustrates a conventional personal computer, including a central processing unit 1850 (“CPU”), a system memory 1805, including a random-access memory 1810 (“RAM”) and a read-only memory (“ROM”) 1815, and a system bus 1835 that couples the system memory 1805 to the CPU 1850. A basic input/output system containing the basic routines that help to transfer information between elements within the computer, such as during startup, is stored in the ROM 1815. The computer 1800 further includes a storage device 1820 for storing an operating system 1825, application/program 1830, and data.

The storage device 1820 is connected to the CPU 1850 through a storage controller (not shown) connected to the bus 1835. The storage device 1820 and its associated computer-readable media, provide non-volatile storage for the computer 1800. Although the description of computer-readable media contained herein refers to a storage device, such as a hard disk or CD-ROM drive, it should be appreciated by those skilled in the art that computer-readable media can be any available media that can be accessed by the computer 1800.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1800 may operate in a networked environment using logical connections to remote computers through a network 1840, such as TCP/IP network such as the Internet or an intranet. The computer 1800 may connect to the network 1840 through a network interface unit 1845 connected to the bus 1835. It should be appreciated that the network interface unit 1845 may also be utilized to connect to other types of networks and remote computer systems.

The computer 1800 may also include an input/output controller 1855 for receiving and processing input from a number of input/output devices 1860, including a keyboard, a mouse, a touchscreen, a camera, a microphone, a controller, a joystick, or other type of input device. Similarly, the input/output controller 1855 may provide output to a display screen, a printer, a speaker, or other type of output device. The computer 1800 can connect to the input/output device 1860 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1820 and RAM 1810 of the computer 1800, including an operating system 1825 suitable for controlling the operation of a networked computer. The storage device 1820 and RAM 1810 may also store one or more applications/programs 1830. In particular, the storage device 1820 and RAM 1810 may store an application/program 1830 for providing a variety of functionalities to a user. For instance, the application/program 1830 may comprise many types of programs such as a word processing application, a spreadsheet application, a desktop publishing application, a database application, a gaming application, internet browsing application, electronic mail application, messaging application, and the like. According to an embodiment of the present invention, the application/program 1830 comprises a multiple functionality software application for providing word processing functionality, slide presentation functionality, spreadsheet functionality, database functionality and the like.

The computer 1800 in some embodiments can include a variety of sensors 1865 for monitoring the environment surrounding and the environment internal to the computer 1800. These sensors 1865 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

The following publications are each hereby incorporated herein by reference in their entirety:
1.) L. G. Eaton and D. C. Funder, “Emotional experience in daily life: valence, variability, and rate of change,” Emotion, vol. 1, pp. 413-421, December 2001.
2.) J. A. Russell, “A circumplex model of affect,” Journal of personality and social psychology, vol. 39, no. 6, p. 1161, 1980.
3.) D. S. Wickramasuriya and R. T. Faghih, “A marked point process filtering approach for tracking sympathetic arousal from skin conductance,” IEEE Access, vol. 8, pp. 68499-68513, 2020.
4.) D. S. Wickramasuriya and R. T. Faghih, “A mixed filter algorithm for sympathetic arousal tracking from skin conductance and heart rate measurements in pavlovian fear conditioning,” PloS one, vol. 15, no. 4, p. e0231659, 2020.
5.) W. Sato, T. Kochiyama, and S. Yoshikawa, “Physiological correlates of subjective emotional valence and arousal dynamics while viewing films,” Biological Psychology, vol. 157, p. 107974, 2020.
6.) L. A. Schmidt and L. J. Trainor, “Frontal brain electrical activity (eeg) distinguishes valence and intensity of musical emotions,” Cognition & Emotion, vol. 15, no. 4, pp. 487-500, 2001.
7.) V. Roy, P. K. Shukla, A. K. Gupta, V. Goel, P. K. Shukla, and S. Shukla, “Taxonomy on EEG artifacts removal methods, issues, and healthcare applications,” J. Organ. End User Comput., vol. 33, pp. 19-46, January 2021.
8.) Q. Zhang, X. Chen, Q. Zhan, T. Yang, and S. Xia, “Respiration-based emotion recognition with deep learning,” Comput. Ind., vol. 92-93, pp. 84-90, November 2017.
9.) M. Soleymani, J. Lichtenauer, T. Pun, and M. Pantic, “A multimodal database for affect recognition and implicit tagging,” IEEE Transactions on Affective Computing, vol. 3, pp. 42-55, 2012.
10.) R. Amin and R. T. Faghih, “Physiological characterization of electrodermal activity enables scalable near real-time autonomic nervous system activation inference,” PLOS computational biology, vol. 18, no. 7, p. e1010275, 2022.
11.) L. R. Branco, A. Ehteshami, H. F. Azgomi, and R. T. Faghih, “Closedloop tracking and regulation of emotional valence state from facial electromyogram measurements,” Frontiers in computational neuroscience, vol. 16, p. 747735, 2022.
12.) D. D. Pednekar, M. R. Amin, H. F. Azgomi, K. Aschbacher, L. J. Crofford, and R. T. Faghih, “Characterization of cortisol dysregulation in fibromyalgia and chronic fatigue syndromes: a state-space approach,” IEEE Transactions on Biomedical Engineering, vol. 67, no. 11, pp. 3163-3172, 2020.
13.) T. Yadav, M. M. U. Atique, H. F. Azgomi, J. T. Francis, and R. T. Faghih, “Emotional valence tracking and classification via state-space analysis of facial electromyography,” in 2019 53rd Asilomar Conference on Signals, Systems, and Computers, pp. 2116-2120, IEEE, 2019.
14.) M. B. Ahmadi, A. Craik, H. F. Azgomi, J. T. Francis, J. L. ContrerasVidal, and R. T. Faghih, “Real-time seizure state tracking using two channels: A mixed-filter approach,” in 2019 53rd Asilomar Conference on Signals, Systems, and Computers, pp. 2033-2039, IEEE, 2019.
15.) D. S. Wickramasuriya and R. T. Faghih, “A bayesian filtering approach for tracking arousal from binary and continuous skin conductance features,” IEEE Transactions on Biomedical Engineering, vol. 67, no. 6, pp. 1749-1760, 2019.
16.) D. S. Wickramasuriya and R. T. Faghih, “A novel filter for tracking real-world cognitive stress using multi-time-scale point process observations,” in 2019 41st Annual International Conference of the IEEE Engineering in Medicine and Biology Society (EMBC), pp. 599-602, IEEE, 2019.
17.) D. S. Wickramasuriya, M. Amin, R. T. Faghih, et al., “skin conductance as a viable alternative for closing the deep brain stimulation loop in neuropsychiatric disorders,” Frontiers in neuroscience, vol. 13, p. 780, 2019.
18.) D. S. Wickramasuriya, C. Qi, and R. T. Faghih, “A state-space approach for detecting stress from electrodermal activity,” in 2018 40th Annual International Conference of the IEEE Engineering in Medicine and Biology Society (EMBC), pp. 3562-3567, IEEE, 2018.
19.) A. C. Smith, L. M. Frank, S. Wirth, M. Yanike, D. Hu, Y. Kubota, A. M. Graybiel, W. A. Suzuki, and E. N. Brown, “Dynamic analysis of learning in behavioral experiments,” J. Neurosci., vol. 24, pp. 447-461, January 2004.
20.) A. C. Smith, M. R. Stefani, B. Moghaddam, and E. N. Brown, “Analysis and design of behavioral experiments to characterize population learning,” Journal of Neurophysiology, vol. 93, no. 3, pp. 1776-1792, 2005.
21.) T. P. Coleman, M. Yanike, W. A. Suzuki, and E. N. Brown, “A mixed-filter algorithm for dynamically tracking learning from multiple behavioral and neurophysiological measures,” The dynamic brain: An exploration of neuronal variability and its functional significance, pp. 3-28, 2011.
22.) M. M. Shanechi, R. C. Hu, M. Powers, G. W. Wornell, E. N. Brown, and Z. M. Williams, “Neural population partitioning and a concurrent brain-machine interface for sequential motor function,” Nature neuroscience, vol. 15, no. 12, pp. 1715-1722, 2012.
23.) H. F. Azgomi and R. T. Faghih, “A wearable brain machine interface architecture for regulation of energy in hypercortisolism,” in 2019 53rd Asilomar Conference on Signals, Systems, and Computers, pp. 254-258, IEEE, 2019.
24.) S. Santaniello, G. C. McConnell, J. T. Gale, R. T. Faghih, C. Kemere, J. D. Hilliard, and M. Han, “Towards the next generation of deep brain stimulation therapies: Technological advancements, computational methods, and new targets,” Frontiers in Neuroscience, vol. 15, 2021.
25.) A. G. Steele, S. Parekh, H. F. Azgomi, M. B. Ahmadi, A. Craik, S. Pati, J. T. Francis, J. L. Contreras-Vidal, and R. T. Faghih, “A mixed filtering approach for real-time seizure state tracking using multi-channel electroencephalography data,” IEEE Transactions on Neural Systems and Rehabilitation Engineering, vol. 29, pp. 2037-2045, 2021.
26.) M. M. Shanechi, R. C. Hu, and Z. M. Williams, “A cortical-spinal prosthesis for targeted limb movement in paralysed primate avatars,” Nature communications, vol. 5, no. 1, pp. 1-9, 2014.
27.) S. Khazaei, M. R. Amin, and R. T. Faghih, “Decoding a neurofeedback-modulated cognitive arousal state to investigateperformance regulation by the yerkes-dodson law,” in 2021 43rd Annual International Conference of the IEEE Engineering in Medicine and Biology Society (EMBC), IEEE, 2021.
28.) G. Castegnetti, A. Tzovara, M. Staib, S. Gerster, and D. R. Bach, “Assessing fear learning via conditioned respiratory amplitude responses,” Psychophysiology, vol. 54, pp. 215-223, February 2017.
29.) D. S. Wickramasuriya, C. Qi, and R. T. Faghih, “A state-space approach for detecting stress from electrodermal activity,” in 2018 40th Annual International Conference of the IEEE Engineering in Medicine and Biology Society (EMBC), IEEE, July 2018.
30.) A. C. Smith, L. M. Frank, S. Wirth, M. Yanike, D. Hu, Y. Kubota, A. M. Graybiel, W. A. Suzuki, and E. N. Brown, “Dynamic analysis of learning in behavioral experiments,” J. Neuroscience, vol. 24, no. 2, pp. 447-461, 2004.
31.) J. M. Mendel, Lessons in estimation theory for signal processing, communications, and control. Prentice Hall, March 1995.
32.) E. N. Brown, L. M. Frank, D. Tang, M. C. Quirk, and M. A. Wilson, “A statistical paradigm for neural spike train decoding applied to position prediction from ensemble firing patterns of rat hippocampal place cells,” J. Neurosci., vol. 18, pp. 7411-7425 September 1998.
33.) R. L. Payne and C. Cooper, Emotions at work: Theory, research and applications for management. John Wiley & Sons, 2003.
34.) A. Dzedzickis, A. Kaklauskas, and V. Bucinskas, “Human emotion recognition: Review of sensors and methods,” Sensors, vol. 20, no. 3, p. 592, 2020.
35.) J. A. Russell, “A circumplex model of affect,” Journal of personality and social psychology, vol. 39, no. 6, p. 1161, 1980.
36.) L. G. Eaton and D. C. Funder, “Emotional experience in daily life: valence, variability, and rate of change,” Emotion, vol. 1, pp. 413-421, December 2001.
37.) P. J. Whalen, S. L. Rauch, N. L. Etcoff, S. C. Mclnerney, M. B. Lee, and M. A. Jenike, “Masked presentations of emotional facial expressions modulate amygdala activity without explicit knowledge,” Journal of neuroscience, vol. 18, no. 1, pp. 411-418, 1998.
38.) E. A. Phelps, “Human emotion and memory: interactions of the amygdala and hippocampal complex,” Current opinion in neurobiology, vol. 14, no. 2, pp. 198-202, 2004.
39.) E. Vlemincx, I. Van Diest, and O. Van den Bergh, “Emotion, sighing, and respiratory variability,” Psychophysiology, vol. 52, no. 5, pp. 657-666, 2015.
40.) G. J. Tortora and B. H. Derrickson, Principles of anatomy and physiology. John Wiley & Sons, 2018.
41.) R. A. Mitchell and A. J. Berger, “Neural regulation of respiration,” American Review of Respiratory Disease, vol. 111, no. 2, pp. 206-224, 1975.
42.) I. Homma and Y. Masaoka, “Breathing rhythms and emotions,” Experimental physiology, vol. 93, no. 9, pp. 1011-1021, 2008.
43.) F. H. Wilhelm, R. Gevirtz, and W. T. Roth, “Respiratory dysregulation in anxiety, functional cardiac, and pain disorders: assessment, phenomenology, and treatment,” Behavior Modification, vol. 25, no. 4, pp. 513-545, 2001.
44.) M. Egger, M. Ley, and S. Hanke, “Emotion recognition from physiological signal analysis: A review,” Electronic Notes in Theoretical Computer Science, vol. 343, pp. 35-55, 2019.
45.) R. De Fazio, M. Stabile, M. De Vittorio, R. Velazquez, and P. Visconti, “An overview of wearable piezoresistive and inertial sensors for respiration rate monitoring,” Electronics, vol. 10, no. 17, p. 2178, 2021.
46.) E. Kirkman, “Respiration: control of ventilation,” Anaesthesia & Intensive Care Medicine, vol. 15, no. 11, pp. 540-543, 2014.
47.) T. M. Leyro, M. V. Versella, M.-J. Yang, H. R. Brinkman, D. L. Hoyt, and P. Lehrer, “Respiratory therapy for the treatment of anxiety: Metaanalytic review and regression,” Clinical psychology review, vol. 84, p. 101980, 2021.
48.) M. Soleymani, J. Lichtenauer, T. Pun, and M. Pantic, “A multimodal database for affect recognition and implicit tagging,” IEEE Transactions on Affective Computing, vol. 3, pp. 42-55, 2012.
49.) S. Koelstra, C. Muhl, M. Soleymani, J.-S. Lee, A. Yazdani, T. Ebrahimi, T. Pun, A. Nijholt, and I. Patras, “Deap: A database for emotion analysis; using physiological signals,” IEEE transactions on affective computing, vol. 3, no. 1, pp. 18-31, 2011.
50.) O. Balan, G. Moise, L. Petrescu, A. Moldoveanu, M. Leordeanu, and F. Moldoveanu, “Emotion classification based on biophysical signals and machine learning techniques,” Symmetry, vol. 12, no. 1, p. 21, 2019.
51.) Q. Zhang, X. Chen, Q. Zhan, T. Yang, and S. Xia, “Respiration-based emotion recognition with deep learning,” Comput. Ind., vol. 92-93, pp. 84-90, November 2017.
52.) O. Bazgir, Z. Mohammadi, and S. A. H. Habibi, “Emotion recognition with machine learning using eeg signals,” in 2018 25th national and 3rd international iranian conference on biomedical engineering (ICBME), pp. 1-5, IEEE, 2018.
53.) J. A. Dom′inguez-Jimenez, K. C. Campo-Landines, J. C. Mart′inezSantos, E. J. Delahoz, and S. H. Contreras-Ortiz, “A machine learning model for emotion recognition from physiological signals,” Biomedical signal processing and control, vol. 55, p. 101646, 2020.
54.) J. Zhang, Z. Yin, P. Chen, and S. Nichele, “Emotion recognition using multi-modal data and machine learning techniques: A tutorial and review,” Information Fusion, vol. 59, pp. 103-126, 2020.
55.) G. Balasubramanian, A. Kanagasabai, J. Mohan, and N. G. Seshadri, “Music induced emotion using wavelet packet decomposition—an eeg study,” Biomedical Signal Processing and Control, vol. 42, pp. 115-128, 2018.
56.) H. Liu, J. Allen, D. Zheng, and F. Chen, “Recent development of respiratory rate measurement technologies,” Physiological measurement, vol. 40, no. 7, p. 07TR01, 2019.
57.) V. Roy, P. K. Shukla, A. K. Gupta, V. Goel, P. K. Shukla, and S. Shukla, “Taxonomy on EEG artifacts removal methods, issues, and healthcare applications,” J. Organ. End User Comput., vol. 33, pp. 19-46, January 2021.
58.) R. Amin and R. T. Faghih, “Physiological characterization of electrodermal activity enables scalable near real-time autonomic nervous system activation inference,” PLOS computational biology, vol. 18, no. 7, p. e1010275, 2022.
59.) L. R. Branco, A. Ehteshami, H. F. Azgomi, and R. T. Faghih, “Closedloop tracking and regulation of emotional valence state from facial electromyogram measurements,” Frontiers in computational neuroscience, vol. 16, p. 747735, 2022.
60.) D. D. Pednekar, M. R. Amin, H. F. Azgomi, K. Aschbacher, L. J. Crofford, and R. T. Faghih, “Characterization of cortisol dysregulation in fibromyalgia and chronic fatigue syndromes: a state-space approach,” IEEE Transactions on Biomedical Engineering, vol. 67, no. 11, pp. 3163-3172, 2020.
61.) T. Yadav, M. M. U. Atique, H. F. Azgomi, J. T. Francis, and R. T. Faghih, “Emotional valence tracking and classification via state-space analysis of facial electromyography,” in 2019 53rd Asilomar Conference on Signals, Systems, and Computers, pp. 2116-2120, IEEE, 2019.
62.) M. B. Ahmadi, A. Craik, H. F. Azgomi, J. T. Francis, J. L. ContrerasVidal, and R. T. Faghih, “Real-time seizure state tracking using two channels: A mixed-filter approach,” in 2019 53rd Asilomar Conference on Signals, Systems, and Computers, pp. 2033-2039, IEEE, 2019.
63.) D. S. Wickramasuriya and R. T. Faghih, “A bayesian filtering approach for tracking arousal from binary and continuous skin conductance features,” IEEE Transactions on Biomedical Engineering, vol. 67, no. 6, pp. 1749-1760, 2019.
64.) D. S. Wickramasuriya and R. T. Faghih, “A novel filter for tracking real-world cognitive stress using multi-time-scale point process observations,” in 2019 41st Annual International Conference of the IEEE Engineering in Medicine and Biology Society (EMBC), pp. 599-602, IEEE, 2019.
65.) D. S. Wickramasuriya, M. Amin, R. T. Faghih, et al., “skin conductance as a viable alternative for closing the deep brain stimulation loop in neuropsychiatric disorders,” Frontiers in neuroscience, vol. 13, p. 780, 2019.
66.) D. S. Wickramasuriya, C. Qi, and R. T. Faghih, “A state-space approach for detecting stress from electrodermal activity,” in 2018 40th Annual International Conference of the IEEE Engineering in Medicine and Biology Society (EMBC), pp. 3562-3567, IEEE, 2018.
67.) A. C. Smith, L. M. Frank, S. Wirth, M. Yanike, D. Hu, Y. Kubota, A. M. Graybiel, W. A. Suzuki, and E. N. Brown, “Dynamic analysis of learning in behavioral experiments,” J. Neurosci., vol. 24, pp. 447-461, January 2004.
68.) A. C. Smith, M. R. Stefani, B. Moghaddam, and E. N. Brown, “Analysis and design of behavioral experiments to characterize population learning,” Journal of Neurophysiology, vol. 93, no. 3, pp. 1776-1792, 2005.
69.) T. P. Coleman, M. Yanike, W. A. Suzuki, and E. N. Brown, “A mixed-filter algorithm for dynamically tracking learning from multiple behavioral and neurophysiological measures,” The dynamic brain: An exploration of neuronal variability and its functional significance, pp. 3-28, 2011.
70.) M. M. Shanechi, R. C. Hu, M. Powers, G. W. Wornell, E. N. Brown, and Z. M. Williams, “Neural population partitioning and a concurrent brainmachine interface for sequential motor function,” Nature neuroscience, vol. 15, no. 12, pp. 1715-1722, 2012.
71.) H. F. Azgomi and R. T. Faghih, “A wearable brain machine interface architecture for regulation of energy in hypercortisolism,” in 2019 53rd Asilomar Conference on Signals, Systems, and Computers, pp. 254-258, IEEE, 2019.
72.) S. Santaniello, G. C. McConnell, J. T. Gale, R. T. Faghih, C. Kemere, J. D. Hilliard, and M. Han, “Towards the next generation of deep brain stimulation therapies: Technological advancements, computational methods, and new targets,” Frontiers in Neuroscience, vol. 15, 2021.
73.) A. G. Steele, S. Parekh, H. F. Azgomi, M. B. Ahmadi, A. Craik, S. Pati, J. T. Francis, J. L. Contreras-Vidal, and R. T. Faghih, “A mixed filtering approach for real-time seizure state tracking using multi-channel electroencephalography data,” IEEE Transactions on Neural Systems and Rehabilitation Engineering, vol. 29, pp. 2037-2045, 2021.
74.) M. M. Shanechi, R. C. Hu, and Z. M. Williams, “A cortical-spinal prosthesis for targeted limb movement in paralysed primate avatars,” Nature communications, vol. 5, no. 1, pp. 1-9, 2014.
75.) S. Khazaei, M. R. Amin, and R. T. Faghih, “Decoding a neurofeedbackmodulated cognitive arousal state to investigateperformance regulation by the yerkes-dodson law,” in 2021 43rd Annual International Conference of the IEEE Engineering in Medicine and Biology Society (EMBC), IEEE, 2021.
76.) D. S. Wickramasuriya and R. T. Faghih, “A marked point process filtering approach for tracking sympathetic arousal from skin conductance,” IEEE Access, vol. 8, pp. 68499-68513, 2020.
77.) G. Castegnetti, A. Tzovara, M. Staib, S. Gerster, and D. R. Bach, “Assessing fear learning via conditioned respiratory amplitude responses,” Psychophysiology, vol. 54, pp. 215-223, February 2017.
78.) W. Sato, T. Kochiyama, and S. Yoshikawa, “Physiological correlates of subjective emotional valence and arousal dynamics while viewing films,” Biological Psychology, vol. 157, p. 107974, 2020.
79.) E. N. Bruce, “Temporal variations in the pattern of breathing,” Journal of Applied Physiology, vol. 80, no. 4, pp. 1079-1087, 1996.
80.) R. Jerath and C. Beveridge, “Respiratory rhythm, autonomic modulation, and the spectrum of emotions: the future of emotion recognition and modulation,” Frontiers in Psychology, vol. 11, p. 1980, 2020.
81.) W. A. Tol, C. Barbui, and M. Van Ommeren, “Management of acute stress, ptsd, and bereavement: Who recommendations,” Jama, vol. 310, no. 5, pp. 477-478, 2013.
82.) M. J. Prerau, A. C. Smith, U. T. Eden, Y. Kubota, M. Yanike, W. Suzuki, A. M. Graybiel, and E. N. Brown, “Characterizing learning by simultaneous analysis of continuous and binary measures of performance,” Journal of neurophysiology, vol. 102, no. 5, pp. 3060-3072, 2009.
83.) J. M. Mendel, Lessons in estimation theory for signal processing, communications, and control. Prentice Hall, March 1995.
84.) E. N. Brown, L. M. Frank, D. Tang, M. C. Quirk, and M. A. Wilson, “A statistical paradigm for neural spike train decoding applied to position prediction from ensemble firing patterns of rat hippocampal place cells,” J. Neurosci., vol. 18, pp. 7411-7425 September 1998.
85.) A. C. Smith, L. M. Frank, S. Wirth, M. Yanike, D. Hu, Y. Kubota, A. M. Graybiel, W. A. Suzuki, and E. N. Brown, “Dynamic analysis of learning in behavioral experiments,” J. Neuroscience, vol. 24, no. 2, pp. 447-461, 2004.
86.) A. P. Dempster, N. M. Laird, and D. B. Rubin, “Maximum likelihood from incomplete data via the em algorithm,” Journal of the royal statistical society: series B (methodological), vol. 39, no. 1, pp. 1-22, 1977.
87.) J. Goheen, J. A. Anderson, J. Zhang, and G. Northoff, “From lung to brain: Respiration modulates neural and mental activity,” Neuroscience Bulletin, pp. 1-14, 2023.
88.) D. J. Meredith, D. Clifton, P. Charlton, J. Brooks, C. Pugh, and L. Tarassenko, “Photoplethysmographic derivation of respiratory rate: a review of relevant physiology,” Journal of medical engineering & technology, vol. 36, no. 1, pp. 1-7, 2012.
89.) L. Devillers, L. Vidrascu, and L. Lamel, “Challenges in real-life emotion annotation and machine learning based detection,” Neural Networks, vol. 18, no. 4, pp. 407-422, 2005.
90.) U.S. Patent App. No. 20220142556
91.) U.S. Patent App. No. 20070066916
92.) U.S. Pat. No. 10,863,939
93.) U.S. Pat. No. 9,503,786
94.) U.S. Pat. No. 10,517,521
95.) China Patent No. CN107080546B
96.) Nilsson, Lena M. MD, PhD. Respiration Signals from Photoplethysmography. Anesthesia & Analgesia 117 (4): p 859-865, October 2013.|DOI: 10.1213/ANE.0b013e31828098b2
97.) R. L. Payne and C. Cooper, Emotions at work: Theory, research and applications for management. John Wiley & Sons, 2003.
98.) R. Reddy et al., “A point-process approach for tracking valence using a respiration belt,” in 2023 45th Annual International Conference of the IEEE Engineering in Medicine & Biology Society (EMBC), pp. 1-7, IEEE, 2023.
99.) Kupfer, D. J., Frank, E., & Phillips, M. L. (2012). Major depressive disorder: new clinical, neurobiological, and treatment perspectives. The Lancet, 379 (9820), 1045-1055.
100.) Hiemke, C., & Härtter, S. (2000). Pharmacokinetics of selective serotonin reuptake inhibitors. Pharmacology & therapeutics, 85 (1), 11-28.
101.) Keller, M. B. (1999). The long-term treatment of depression. Journal of Clinical Psychiatry, 60, 41-45.
102.) Jerath, R., Crawford, M. W., Barnes, V. A., & Harden, K. (2015). Self-regulation of breathing as a primary treatment for anxiety. Applied psychophysiology and biofeedback, 40 (2), 107-115.
103.) Aalbers, S., Fusar-Poli, L., Freeman, R. E., Spreen, M., Ket, J. C., Vink, A. C., . . . & Gold, C. (2017). Music therapy for depression. Cochrane database of systematic reviews, (11).
104.) Koelsch, S., Offermanns, K., & Franzke, P. (2010). Music in the treatment of affective disorders: an exploratory investigation of a new method for music-therapeutic research. Music Perception, 27 (4), 307-316.
105.) Khazaei, S., Parshi, S., Alam, S., Amin, M. R., & Faghih, R. T. (2024). A multimodal dataset for investigating working memory in presence of music: a pilot study. Frontiers in Neuroscience, 18, 1406814.
106.) Fekri Azgomi, H., F. Branco, L. R., Amin, M. R., Khazaei, S., & Faghih, R. T. (2023). Regulation of brain cognitive states through auditory, gustatory, and olfactory stimulation with wearable monitoring. Scientific reports, 13 (1), 12399.
107.) Hiraba, Hisao, et al. “Facial Vibrotactile Stimulation Activates the Parasympathetic Nervous System: Study of Salivary Secretion, Heart Rate, Pupillary Reflex, and Functional Near-Infrared Spectroscopy Activity.” BioMed research international 2014.1 (2014): 910812.
108.) Tyler, William J., et al. “Transdermal neuromodulation of noradrenergic activity suppresses psychophysiological and biochemical stress responses in humans.” Scientific reports 5.1 (2015): 13865.
109.) Ernesto Cesar Pinto Leal-Junior, Heliodora Leão Casalechi, Caroline dos Santos Monteiro Machado, Amy Serin, Nathan S. Hageman et al. (2019) A Triple-Blind, Placebo-Controlled Randomized Trial of the Effect of Bilateral Alternating Somatosensory Stimulation on Reducing Stress-Related Cortisol and Anxiety During and After the Trier Social Stress Test. Journal of Biotechnology and Biomedical Science-2 (1): 22-30. https://doi.org/10.14302/issn.2576-6694.jbbs-19-2784.
110.) Nilsson, L. M. (2013). Respiration signals from photoplethysmography. Anesthesia & Analgesia, 117 (4), 859-865.
111.) Rehouma, H., Noumeir, R., Essouri, S., & Jouvet, P. (2020). Advancements in methods and camera-based sensors for the quantification of respiration. Sensors, 20 (24), 7252.
112.) Perciavalle, Valentina, et al. “The role of deep breathing on stress.” Neurological Sciences 38.3 (2017): 451-458.
113.) Steffen, Patrick R., et al. “The impact of resonance frequency breathing on measures of heart rate variability, blood pressure, and mood.” Frontiers in public health 5 (2017): 222.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A system for estimating emotional valence continuously based on physiological measurements of respiration activity, comprising:
a respiration sensor;
a low-performance computing device configured to acquire the sensor data and estimate an emotional valence state of the wearer via an emotional valence estimator;
a high-performance computing device configured to provide feedback to the low-performance computing device in order to improve valence estimation; and
a display to show the estimated valence level.

2. The system of claim 1, wherein at least one of the low-performance computing device and the high-performance computing device comprises a processor and a non-transitory computer-readable medium with instructions stored thereon, which when executed by the processor, perform steps comprising:
measuring a respiration signal via the respiration sensor;
calculating a depth of breath, breathing cycle time, and respiration rate based on the respiration signal;
generating a marked point process (MPP) using k-means grouping of the calculated depth of breath, breathing cycle time, and respiration rate;
estimating a valence level based on the MPP; and
displaying the estimated valence level.

3. The system of claim 1, where the respiration sensor comprises a respiration belt, a camera, an electroencephalogram (EEG), or an electrocardiogram (ECG).

4. The system of claim 1, further comprising a stimulation device configured to perform an intervention.

5. The system of claim 4, wherein the intervention comprises a vibration.

6. The system of claim 4, wherein the intervention comprises an electrical stimulation.

7. The system of claim 1, wherein the display is configured to display an intervention suggestion.

8. The system of claim 7, wherein the intervention suggestion comprises instructions to perform a breathing exercise, playing music, or instructions to administer medication.

9. A method for estimating emotional valence continuously based on physiological measurements of respiration activity, comprising:
measuring a respiration signal via a respiration sensor;
calculating a depth of breath, breathing cycle time, and respiration rate based on the respiration signal;
generating a marked point process (MPP) using a k-means grouping algorithm on the calculated depth of breath, breathing cycle time, and respiration rate;
estimating a valence level based on the MPP; and
displaying the estimated valence level.

10. The method of claim 9, further comprising improving the valence estimation via feedback.

11. The method of claim 9, wherein the step of generating the MPP comprises identifying high and low valence events in the respiration signal.

12. The method of claim 11, wherein the high and low valence events are identified by comparing features extracted from each breath to their expected behavior during no emotional response.

13. The method of claim 9, wherein the respiration signal comprises a waveform including inhalation amplitude, exhalation amplitude, inhalation time, and exhalation time.

14. The method of claim 9, wherein the depth of breath is the difference between the amplitude of respiration measured at the end of inhalation and the amplitude measured at the start of inhalation, and the rate of inhalation comprises breath amplitude divided by the time of inhalation.

15. The method of claim 9, wherein the algorithm comprises an unsupervised algorithm.

16. The method of claim 15, wherein the unsupervised algorithm comprises k-means clustering.

17. The method of claim 9, further comprising administering an intervention when a negative valence is estimated.

18. The method of claim 17, wherein the intervention comprises instructions to perform a breathing exercise, music, or instructions to administer medication.

19. The method of claim 17, wherein the intervention is automated.

20. The method of claim 17, wherein the intervention comprises a vibration or an electrostimulation.
